SEQUENCE LISTING (236 sequences)

SEQ ID NO: 1 - protein, 17 aa, Artificial Sequence
  Description of Artificial Sequence: Synthetic peptide
KSSQSLYYSGVKENLLA

SEQ ID NO: 2 - protein, 6 aa, Artificial Sequence
  Description of Artificial Sequence: Synthetic peptide
ASIRFT

SEQ ID NO: 3 - protein, 9 aa, Artificial Sequence
  Description of Artificial Sequence: Synthetic peptide
QQGINNPLT

SEQ ID NO: 4 - protein, 10 aa, Artificial Sequence
  Description of Artificial Sequence: Synthetic peptide
GFTFSSFTMH

SEQ ID NO: 5 - protein, 17 aa, Artificial Sequence
  Description of Artificial Sequence: Synthetic peptide
FIRSGSGIVFYADAVRG

SEQ ID NO: 6 - protein, 10 aa, Artificial Sequence
  Description of Artificial Sequence: Synthetic peptide
RPLGHNTFDS

SEQ ID NO: 7 - protein, 16 aa, Artificial Sequence
  Description of Artificial Sequence: Synthetic peptide
RSSQSLVNSYGNTFLS

SEQ ID NO: 8 - protein, 7 aa, Artificial Sequence
  Description of Artificial Sequence: Synthetic peptide
GISNRFS

SEQ ID NO: 9 - protein, 9 aa, Artificial Sequence
  Description of Artificial Sequence: Synthetic peptide
LQGTHQPPT

SEQ ID NO: 10 - protein, 10 aa, Artificial Sequence
  Description of Artificial Sequence: Synthetic peptide
GYSFTGHLMN

SEQ ID NO: 11 - protein, 17 aa, Artificial Sequence
  Description of Artificial Sequence: Synthetic peptide
LIIPYNGGTSYNQKFKG

SEQ ID NO: 12 - protein, 10 aa, Artificial Sequence
  Description of Artificial Sequence: Synthetic peptide
GLRGFYAMDY

SEQ ID NO: 13 - protein, 114 aa, Artificial Sequence
  Description of Artificial Sequence: Synthetic peptide
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKR

SEQ ID NO: 14 - protein, 112 aa, Artificial Sequence
  Description of Artificial Sequence: Synthetic peptide
DVVLTQTPLSLSVSFGDQVSISCRSSQSLVNSYGNTFLSWYLHKPGQSPQLLIFGISNRFSGVPDRFSGSGSGTDFTLKISTIKPEDLGMYYCLQGTHQPPTFGPGTKLEVK

SEQ ID NO: 15 - protein, 119 aa, Artificial Sequence
  Description of Artificial Sequence: Synthetic peptide
EVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSS

SEQ ID NO: 16 - protein, 119 aa, Artificial Sequence
  Description of Artificial Sequence: Synthetic peptide
EVQLQQSGPELVKPGTSMKISCKASGYSFTGHLMNWVKQSHGKNLEWIGLIIPYNGGTSYNQKFKGKATLTVDKSSSTAYMELLSLTSDDSAVYFCSRGLRGFYAMDYWGQGTSVTVSS

SEQ ID NO: 17-20 - skipped (4 entries)

SEQ ID NO: 21 - protein, 249 aa, Homo sapiens
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 22 - protein, 5 aa, Artificial Sequence
  Description of Artificial Sequence: Synthetic peptide
DSYMS

SEQ ID NO: 23 - protein, 17 aa, Artificial Sequence
  Description of Artificial Sequence: Synthetic peptide
DMYPDNGDSSYNQKFRE

SEQ ID NO: 24 - protein, 8 aa, Artificial Sequence
  Description of Artificial Sequence: Synthetic peptide
APRWYFSV

SEQ ID NO: 25 - protein, 11 aa, Artificial Sequence
  Description of Artificial Sequence: Synthetic peptide
RASQDISNYLN

SEQ ID NO: 26 - protein, 7 aa, Artificial Sequence
  Description of Artificial Sequence: Synthetic peptide
YTSRLRS

SEQ ID NO: 27 - protein, 9 aa, Artificial Sequence
  Description of Artificial Sequence: Synthetic peptide
QQGHTLPPT

SEQ ID NO: 28 - protein, 5 aa, Artificial Sequence
  Description of Artificial Sequence: Synthetic peptide
DAYMS

SEQ ID NO: 29 - protein, 5 aa, Artificial Sequence
  Description of Artificial Sequence: Synthetic peptide
ESYMS

SEQ ID NO: 30 - protein, 17 aa, Artificial Sequence
  Description of Artificial Sequence: Synthetic peptide
DMYPDNADSSYNQKFRE

SEQ ID NO: 31 - protein, 17 aa, Artificial Sequence
  Description of Artificial Sequence: Synthetic peptide
DMYPDNADASYNQKFRE

SEQ ID NO: 32 - protein, 17 aa, Artificial Sequence
  Description of Artificial Sequence: Synthetic peptide
DMYPDNGDASYNQKFRE

SEQ ID NO: 33 - protein, 17 aa, Artificial Sequence
  Description of Artificial Sequence: Synthetic peptide
DMYPDSGDSSYNQKFRE

SEQ ID NO: 34 - protein, 17 aa, Artificial Sequence
  Description of Artificial Sequence: Synthetic peptide
DMYPDNGSSSYNQKFRE

SEQ ID NO: 35 - protein, 8 aa, Artificial Sequence
  Description of Artificial Sequence: Synthetic peptide
APRWYFSA

SEQ ID NO: 36 - protein, 8 aa, Artificial Sequence
  Description of Artificial Sequence: Synthetic peptide
APRWYASV

SEQ ID NO: 37 - protein, 8 aa, Artificial Sequence
  Description of Artificial Sequence: Synthetic peptide
APRWAFSV

SEQ ID NO: 38 - protein, 8 aa, Artificial Sequence
  Description of Artificial Sequence: Synthetic peptide
APAWYFSV

SEQ ID NO: 39 - protein, 8 aa, Artificial Sequence
  Description of Artificial Sequence: Synthetic peptide
APRWYFAV

SEQ ID NO: 40 - protein, 8 aa, Artificial Sequence
  Description of Artificial Sequence: Synthetic peptide
APRAYFSV

SEQ ID NO: 41 - protein, 8 aa, Artificial Sequence
  Description of Artificial Sequence: Synthetic peptide
AARWYFSV

SEQ ID NO: 42 - protein, 9 aa, Artificial Sequence
  Description of Artificial Sequence: Synthetic peptide
QQGHTLPAT

SEQ ID NO: 43 - protein, 9 aa, Artificial Sequence
  Description of Artificial Sequence: Synthetic peptide
QQGHTAPPT

SEQ ID NO: 44 - protein, 9 aa, Artificial Sequence
  Description of Artificial Sequence: Synthetic peptide
QQGATLPPT

SEQ ID NO: 45 - protein, 9 aa, Artificial Sequence
  Description of Artificial Sequence: Synthetic peptide
QQGHALPPT

SEQ ID NO: 46 - protein, 9 aa, Artificial Sequence
  Description of Artificial Sequence: Synthetic peptide
QQAHTLPPT

SEQ ID NO: 47 - protein, 9 aa, Artificial Sequence
  Description of Artificial Sequence: Synthetic peptide
QQGHTLAPT

SEQ ID NO: 48 - protein, 9 aa, Artificial Sequence
  Description of Artificial Sequence: Synthetic peptide
QAGHTLPPT

SEQ ID NO: 49 - protein, 5 aa, Artificial Sequence
  Description of Artificial Sequence: Synthetic peptide
NYLIE

SEQ ID NO: 50 - protein, 17 aa, Artificial Sequence
  Description of Artificial Sequence: Synthetic peptide
VINPGSGDTYYSEKFKG

SEQ ID NO: 51 - protein, 17 aa, Artificial Sequence
  Description of Artificial Sequence: Synthetic peptide
VINPGSGDAYYSEKFKG

SEQ ID NO: 52 - protein, 17 aa, Artificial Sequence
  Description of Artificial Sequence: Synthetic peptide
VINPGSGDQYYSEKFKG

SEQ ID NO: 53 - protein, 5 aa, Artificial Sequence
  Description of Artificial Sequence: Synthetic peptide
DRLDY

SEQ ID NO: 54 - protein, 5 aa, Artificial Sequence
  Description of Artificial Sequence: Synthetic peptide
ARLDY

SEQ ID NO: 55 - protein, 5 aa, Artificial Sequence
  Description of Artificial Sequence: Synthetic peptide
DALDY

SEQ ID NO: 56 - protein, 5 aa, Artificial Sequence
  Description of Artificial Sequence: Syntheti c peptide
DRADY

SEQ ID NO: 57 - protein, 11 aa, Artificial Sequence
  Description of Artificial Sequence: Synthetic peptide
HASQDISSYIV

SEQ ID NO: 58 - protein, 7 aa, Artificial Sequence
  Description of Artificial Sequence: Synthetic peptide
HGTNLED

SEQ ID NO: 59 - protein, 7 aa, Artificial Sequence
  Description of Artificial Sequence: Synthetic peptide
HGTNLES

SEQ ID NO: 60 - protein, 7 aa, Artificial Sequence
  Description of Artificial Sequence: Synthetic peptide
HGTNLEE

SEQ ID NO: 61 - protein, 7 aa, Artificial Sequence
  Description of Artificial Sequence: Synthetic peptide
HGTNLEQ

SEQ ID NO: 62 - protein, 9 aa, Artificial Sequence
  Description of Artificial Sequence: Synthetic peptide
VHYAQFPYT

SEQ ID NO: 63 - protein, 9 aa, Artificial Sequence
  Description of Artificial Sequence: Synthetic peptide
AHYAQFPYT

SEQ ID NO: 64 - protein, 9 aa, Artificial Sequence
  Description of Artificial Sequence: Synthetic peptide
VAYAQFPYT

SEQ ID NO: 65 - protein, 9 aa, Artificial Sequence
  Description of Artificial Sequence: Synthetic peptide
VHAAQFPYT

SEQ ID NO: 66 - protein, 9 aa, Artificial Sequence
  Description of Artificial Sequence: Synthetic peptide
VHYAAFPYT

SEQ ID NO: 67 - protein, 9 aa, Artificial Sequence
  Description of Artificial Sequence: Synthetic peptide
VHYAQAPYT

SEQ ID NO: 68 - protein, 9 aa, Artificial Sequence
  Description of Artificial Sequence: Synthetic peptide
VHYAQFAYT

SEQ ID NO: 69 - protein, 9 aa, Artificial Sequence
  Description of Artificial Sequence: Synthetic peptide
VHYAQFPAT

SEQ ID NO: 70 - protein, 5 aa, Artificial Sequence
  Description of Artificial Sequence: Synthetic peptide
DYGVL

SEQ ID NO: 71 - protein, 16 aa, Artificial Sequence
  Description of Artificial Sequence: Synthetic peptide
MIWSGGTTDYNAAFIS

SEQ ID NO: 72 - protein, 5 aa, Artificial Sequence
  Description of Artificial Sequence: Synthetic peptide
EEMDY

SEQ ID NO: 73 - protein, 11 aa, Artificial Sequence
  Description of Artificial Sequence: Synthetic peptide
RASQDISNFLN

SEQ ID NO: 74 - protein, 7 aa, Artificial Sequence
  Description of Artificial Sequence: Synthetic peptide
YTSRLHS

SEQ ID NO: 75 - protein, 9 aa, Artificial Sequence
  Description of Artificial Sequence: Synthetic peptide
QQGNTLPWT

SEQ ID NO: 76 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 77 - protein, 107 aa, Artificial Sequence
  Description of Artificial Sequence: Synthetic peptide
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 78 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGDSSYNQKFRERVTITVDTSTSTAYLELSSLRSEDTAVYYCVLAPRWYFSVWGQGTLVTVSS

SEQ ID NO: 79 - protein, 107 aa, Artificial Sequence; identical to SEQ ID NO: 77
  Description of Artificial Sequence: Synthetic peptide

SEQ ID NO: 80 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGDSSYNQKFRERVTLTVDTSTSTAYLELSSLRSEDTAVYYCVLAPRWYFSVWGQGTLVTVSS

SEQ ID NO: 81 - protein, 107 aa, Artificial Sequence; identical to SEQ ID NO: 77
  Description of Artificial Sequence: Synthetic peptide

SEQ ID NO: 82 - protein, 117 aa, Artificial Sequence; identical to SEQ ID NO: 78
  Description of Artificial Sequence: Synthetic peptide

SEQ ID NO: 83 - protein, 107 aa, Artificial Sequence
  Description of Artificial Sequence: Synthetic peptide
DIQMTQSPSSLSASVGDRVTITCRASQDISNYLNWYQQKPGKTVKLLIYYTSRLRSGVPSRFSGSGSGTDFTLTISSLQPEDFATYYCQQGHTLPPTFGQGTKVEIK

SEQ ID NO: 84 - protein, 117 aa, Artificial Sequence; identical to SEQ ID NO: 78
  Description of Artificial Sequence: Synthetic peptide

SEQ ID NO: 85 - protein, 107 aa, Artificial Sequence; identical to SEQ ID NO: 83
  Description of Artificial Sequence: Synthetic peptide

SEQ ID NO: 86 - protein, 117 aa, Artificial Sequence; identical to SEQ ID NO: 78
  Description of Artificial Sequence: Synthetic peptide

SEQ ID NO: 87 - protein, 107 aa, Artificial Sequence
  Description of Artificial Sequence: Synthetic peptide
DIQMTQSPSSLSASVGDRVTITCRASQDISNYLNWYQQKPGKTVKLLIYYTSRLRSGVPSRFSGSGSGKDYTLTISSLQPEDFATYFCQQGHTLPPTFGQGTKVEIK

SEQ ID NO: 88 - protein, 117 aa, Artificial Sequence; identical to SEQ ID NO: 78
  Description of Artificial Sequence: Synthetic peptide

SEQ ID NO: 89 - protein, 107 aa, Artificial Sequence; identical to SEQ ID NO: 87
  Description of Artificial Sequence: Synthetic peptide

SEQ ID NO: 90 - protein, 117 aa, Artificial Sequence
  Description of Artificial Sequence: Synthetic peptide
EVQLVQSGAEVKKPGASVKVSCKASGYTFTDAYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 91 - protein, 107 aa, Artificial Sequence; identical to SEQ ID NO: 77
  Description of Artificial Sequence: Synthetic peptide

SEQ ID NO: 92 - protein, 117 aa, Artificial Sequence
  Description of Artificial Sequence: Synthetic peptide
EVQLVQSGAEVKKPGASVKVSCKASGYTFTESYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 93 - protein, 107 aa, Artificial Sequence; identical to SEQ ID NO: 77
  Description of Artificial Sequence: Synthetic peptide

SEQ ID NO: 94 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNADSSYNQKFRERVTITRDTSTSTAYLELSSLRSEDTAVYYCVLAPRWYFSVWGQGTLVTVSS

SEQ ID NO: 95 - protein, 107 aa, Artificial Sequence; identical to SEQ ID NO: 77
  Description of Artificial Sequence: Synthetic peptide

SEQ ID NO: 96 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNADASYNQKFRERVTITRDTSTSTAYLELSSLRSEDTAVYYCVLAPRWYFSVWGQGTLVTVSS

SEQ ID NO: 97 - protein, 107 aa, Artificial Sequence; identical to SEQ ID NO: 77
  Description of Artificial Sequence: Synthetic peptide

SEQ ID NO: 98 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGDASYNQKFRERVTITRDTSTSTAYLELSSLRSEDTAVYYCVLAPRWYFSVWGQGTLVTVSS

SEQ ID NO: 99 - protein, 107 aa, Artificial Sequence; identical to SEQ ID NO: 77
  Description of Artificial Sequence: Synthetic peptide

SEQ ID NO: 100 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDSGDSSYNQKFRERVTITRDTSTSTAYLELSSLRSEDTAVYYCVLAPRWYFSVWGQGTLVTVSS

SEQ ID NO: 101 - protein, 107 aa, Artificial Sequence; identical to SEQ ID NO: 77
  Description of Artificial Sequence: Synthetic peptide

SEQ ID NO: 102 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGSSSYNQKFRERVTITRDTSTSTAYLELSSLRSEDTAVYYCVLAPRWYFSVWGQGTLVTVSS

SEQ ID NO: 103 - protein, 107 aa, Artificial Sequence; identical to SEQ ID NO: 77
  Description of Artificial Sequence: Synthetic peptide

SEQ ID NO: 104 - protein, 117 aa, Artificial Sequence
  Description of Artificial Sequence: Synthetic peptide
EVQLVQSGAEVKKPGASVKVSCKASGYTFTDAYMSWVRQAPGQGLEWIGDMYPDNADASYNQKFRERVTITRDTSTSTAYLELSSLRSEDTAVYYCVLAPRWYFSVWGQGTLVTVSS

SEQ ID NO: 105 - protein, 107 aa, Artificial Sequence; identical to SEQ ID NO: 77
  Description of Artificial Sequence: Synthetic peptide

SEQ ID NO: 106 - protein, 117 aa, Artificial Sequence; identical to SEQ ID NO: 76
  Description of Artificial Sequence: Synthetic peptide

SEQ ID NO: 107 - protein, 107 aa, Artificial Sequence
  Description of Artificial Sequence: Synthetic peptide
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPATFGQGTKVEIK

SEQ ID NO: 108 - protein, 117 aa, Artificial Sequence; identical to SEQ ID NO: 76
  Description of Artificial Sequence: Synthetic peptide

SEQ ID NO: 109 - protein, 107 aa, Artificial Sequence
  Description of Artificial Sequence: Synthetic peptide
DIQMTQSPSSLSASVGDRVTITCRASQDISNYLNWYQQKPGKAPKLLIYYTSRLRSGVPSRFSGSGSGTDFTLTISSLQPEDFATYYCQQGHTAPPTFGQGTKVEIK

SEQ ID NO: 110 - protein, 117 aa, Artificial Sequence; identical to SEQ ID NO: 76
  Description of Artificial Sequence: Synthetic peptide

SEQ ID NO: 111 - protein, 107 aa, Artificial Sequence
  Description of Artificial Sequence: Synthetic peptide
DIQMTQSPSSLSASVGDRVTITCRASQDISNYLNWYQQKPGKAPKLLIYYTSRLRSGVPSRFSGSGSGTDFTLTISSLQPEDFATYYCQQGATLPPTFGQGTKVEIK

SEQ ID NO: 112 - protein, 117 aa, Artificial Sequence; identical to SEQ ID NO: 76
  Description of Artificial Sequence: Synthetic peptide

SEQ ID NO: 113 - protein, 107 aa, Artificial Sequence
  Description of Artificial Sequence: Synthetic peptide
DIQMTQSPSSLSASVGDRVTITCRASQDISNYLNWYQQKPGKAPKLLIYYTSRLRSGVPSRFSGSGSGTDFTLTISSLQPEDFATYYCQQGHALPPTFGQGTKVEIK

SEQ ID NO: 114 - protein, 117 aa, Artificial Sequence; identical to SEQ ID NO: 76
  Description of Artificial Sequence: Synthetic peptide

SEQ ID NO: 115 - protein, 107 aa, Artificial Sequence
  Description of Artificial Sequence: Synthetic peptide
DIQMTQSPSSLSASVGDRVTITCRASQDISNYLNWYQQKPGKAPKLLIYYTSRLRSGVPSRFSGSGSGTDFTLTISSLQPEDFATYYCQQAHTLPPTFGQGTKVEIK

SEQ ID NO: 116 - protein, 117 aa, Artificial Sequence; identical to SEQ ID NO: 76
  Description of Artificial Sequence: Synthetic peptide

SEQ ID NO: 117 - protein, 107 aa, Artificial Sequence
  Description of Artificial Sequence: Synthetic peptide
DIQMTQSPSSLSASVGDRVTITCRASQDISNYLNWYQQKPGKAPKLLIYYTSRLRSGVPSRFSGSGSGTDFTLTISSLQPEDFATYYCQQGHTLAPTFGQGTKVEIK

SEQ ID NO: 118 - protein, 117 aa, Artificial Sequence; identical to SEQ ID NO: 76
  Description of Artificial Sequence: Synthetic peptide

SEQ ID NO: 119 - protein, 107 aa, Artificial Sequence
  Description of Artificial Sequence: Synthetic peptide
DIQMTQSPSSLSASVGDRVTITCRASQDISNYLNWYQQKPGKAPKLLIYYTSRLRSGVPSRFSGSGSGTDFTLTISSLQPEDFATYYCQAGHTLPPTFGQGTKVEIK

SEQ ID NO: 120 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSAWGQGTLVTVSS

SEQ ID NO: 121 - protein, 107 aa, Artificial Sequence; identical to SEQ ID NO: 77
  Description of Artificial Sequence: Synthetic peptide

SEQ ID NO: 122 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYASVWGQGTLVTVSS

SEQ ID NO: 123 - protein, 107 aa, Artificial Sequence; identical to SEQ ID NO: 77
  Description of Artificial Sequence: Synthetic peptide

SEQ ID NO: 124 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWAFSVWGQGTLVTVSS

SEQ ID NO: 125 - protein, 107 aa, Artificial Sequence; identical to SEQ ID NO: 77
  Description of Artificial Sequence: Synthetic peptide

SEQ ID NO: 126 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPAWYFSVWGQGTLVTVSS

SEQ ID NO: 127 - protein, 107 aa, Artificial Sequence; identical to SEQ ID NO: 77
  Description of Artificial Sequence: Synthetic peptide

SEQ ID NO: 128 - protein, 117 aa, Artificial Sequence
  escription of Artificial Sequence: Synthetic peptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFAVWGQGTLVTVSS

SEQ ID NO: 129 - protein, 107 aa, Artificial Sequence; identical to SEQ ID NO: 77
  Description of Artificial Sequence: Synthetic peptide

SEQ ID NO: 130 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRAYFSVWGQGTLVTVSS

SEQ ID NO: 131 - protein, 107 aa, Artificial Sequence; identical to SEQ ID NO: 77
  Description of Artificial Sequence: Synthetic peptide

SEQ ID NO: 132 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAARWYFSVWGQGTLVTVSS

SEQ ID NO: 133 - protein, 107 aa, Artificial Sequence; identical to SEQ ID NO: 77
  Description of Artificial Sequence: Synthetic peptide

SEQ ID NO: 134 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCALAPRWYFSVWGQGTLVTVSS

SEQ ID NO: 135 - protein, 107 aa, Artificial Sequence; identical to SEQ ID NO: 77
  Description of Artificial Sequence: Synthetic peptide

SEQ ID NO: 136 - protein, 117 aa, Artificial Sequence
  Description of Artificial Sequence: Synthetic peptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVAAPRWYFSVWGQGTLVTVSS

SEQ ID NO: 137 - protein, 107 aa, Artificial Sequence; identical to SEQ ID NO: 77
  Description of Artificial Sequence: Synthetic peptide

SEQ ID NO: 138 - protein, 114 aa, Artificial Sequence
  Description of Artificial Sequence: Synthetic peptide
EVQLVQSGAEVKKPGASVKVSCKASGYAFTNYLIEWVRQAPGQGLEWIGVINPGSGDTYYSEKFKGRVTITRDTSTSTAYLELSSLRSEDTAVYYCARDRLDYWGQGTLVTVSS

SEQ ID NO: 139 - protein, 107 aa, Artificial Sequence
  Description of Artificial Sequence: Synthetic peptide
DIQMTQSPSSLSASVGDRVTITCHASQDISSYIVWYQQKPGKAPKLLIYHGTNLEDGVPSRFSGSGSGTDFTLTISSLQPEDFATYYCVHYAQFPYTFGQGTKVEIK

SEQ ID NO: 140 - protein, 114 aa, Artificial Sequence
  Description of Artificial Sequence: Synthetic peptide
EVQLVQSGAEVKKPGASVKVSCKASGYAFTNYLIEWVRQAPGQGLEWIGVINPGSGDTYYSEKFKGRVTITADTSTSTAYLELSSLRSEDTAVYYCARDRLDYWGQGTLVTVSS

SEQ ID NO: 141 - protein, 107 aa, Artificial Sequence; identical to SEQ ID NO: 139
  Description of Artificial Sequence: Synthetic peptide

SEQ ID NO: 142 - protein, 114 aa, Artificial Sequence
  Description of Artificial Sequence: Synthetic peptide
EVQLVQSGAEVKKPGASVKVSCKASGYAFTNYLIEWVRQAPGQGLEWIGVINPGSGDTYYSEKFKGRVTLTADTSTSTAYLELSSLRSEDTAVYYCARDRLDYWGQGTLVTVSS

SEQ ID NO: 143 - protein, 107 aa, Artificial Sequence; identical to SEQ ID NO: 139
  Description of Artificial Sequence: Synthetic peptide

SEQ ID NO: 144 - protein, 114 aa, Artificial Sequence; identical to SEQ ID NO: 140
  Description of Artificial Sequence: Synthetic peptide

SEQ ID NO: 145 - protein, 107 aa, Artificial Sequence
  Description of Artificial Sequence: Synthetic peptide
DIQMTQSPSSLSASVGDRVTITCHASQDISSYIVWYQQKPGKSFKGLIYHGTNLEDGVPSRFSGSGSGTDFTLTISSLQPEDFATYYCVHYAQFPYTFGQGTKVEIK

SEQ ID NO: 146 - protein, 114 aa, Artificial Sequence; identical to SEQ ID NO: 142
  Description of Artificial Sequence: Synthetic peptide

SEQ ID NO: 147 - protein, 107 aa, Artificial Sequence; identical to SEQ ID NO: 145
  Description of Artificial Sequence: Synthetic peptide

SEQ ID NO: 148 - protein, 114 aa, Artificial Sequence; identical to SEQ ID NO: 142
  Description of Artificial Sequence: Synthetic peptide

SEQ ID NO: 149 - protein, 107 aa, Artificial Sequence
  Description of Artificial Sequence: Synthetic peptide
DIQMTQSPSSLSASVGDRVTITCHASQDISSYIVWYQQKPGKSFKGLIYHGTNLESGVPSRFSGSGSGTDFTLTISSLQPEDFATYYCVHYAQFPYTFGQGTKVEIK

SEQ ID NO: 150 - protein, 114 aa, Artificial Sequence; identical to SEQ ID NO: 142
  Description of Artificial Sequence: Synthetic peptide

SEQ ID NO: 151 - protein, 107 aa, Artificial Sequence
  Description of Artificial Sequence: Synthetic peptide
DIQMTQSPSSLSASVGDRVTITCHASQDISSYIVWYQQKPGKSFKGLIYHGTNLEEGVPSRFSGSGSGTDFTLTISSLQPEDFATYYCVHYAQFPYTFGQGTKVEIK

SEQ ID NO: 152 - protein, 114 aa, Artificial Sequence; identical to SEQ ID NO: 142
  Description of Artificial Sequence: Synthetic peptide

SEQ ID NO: 153 - protein, 107 aa, Artificial Sequence
  Description of Artificial Sequence: Synthetic peptide
DIQMTQSPSSLSASVGDRVTITCHASQDISSYIVWYQQKPGKSFKGLIYHGTNLEQGVPSRFSGSGSGTDFTLTISSLQPEDFATYYCVHYAQFPYTFGQGTKVEIK

SEQ ID NO: 154 - protein, 114 aa, Artificial Sequence; identical to SEQ ID NO: 140
  Description of Artificial Sequence: Synthetic peptide

SEQ ID NO: 155 - protein, 107 aa, Artificial Sequence
  Description of Artificial Sequence: Synthetic peptide
DIQMTQSPSSLSASVGDRVTITCHASQDISSYIVWYQQKPGKSFKGLIYHGTNLEDGVPSRFSGSGSGADYTLTISSLQPEDFATYYCVHYAQFPYTFGQGTKVEIK

SEQ ID NO: 156 - protein, 114 aa, Artificial Sequence; identical to SEQ ID NO: 142
  Description of Artificial Sequence: Synthetic peptide

SEQ ID NO: 157 - protein, 107 aa, Artificial Sequence; identical to SEQ ID NO: 155
  Description of Artificial Sequence: Synthetic peptide

SEQ ID NO: 158 - protein, 114 aa, Artificial Sequence
  Description of Artificial Sequence: Synthetic peptide
EVQLVQSGAEVKKPGASVKVSCKASGYAFTNYLIEWVRQAPGQGLEWIGVINPGSGDTYYSEKFKGRVTLTRDTSTSTAYLELSSLRSEDTAVYYCARDRLDYWGQGTLVTVSS

SEQ ID NO: 159 - protein, 107 aa, Artificial Sequence; identical to SEQ ID NO: 145
  Description of Artificial Sequence: Synthetic peptide

SEQ ID NO: 160 - protein, 114 aa, Artificial Sequence; identical to SEQ ID NO: 158
  Description of Artificial Sequence: Synthetic peptide

SEQ ID NO: 161 - protein, 107 aa, Artificial Sequence
  Description of Artificial Sequence: Synthetic peptide
DIQMTQSPSSLSASVGDRVTITCHASQDISSYIVWYQQKPGKSPKLLIYHGTNLEDGVPSRFSGSGSGTDFTLTISSLQPEDFATYYCVHYAQFPYTFGQGTKVEIK

SEQ ID NO: 162 - protein, 114 aa, Artificial Sequence; identical to SEQ ID NO: 158
  Description of Artificial Sequence: Synthetic peptide

SEQ ID NO: 163 - protein, 107 aa, Artificial Sequence
  Description of Artificial Sequence: Synthetic peptide
DIQMTQSPSSLSASVGDRVTITCHASQDISSYIVWYQQKPGKAFKLLIYHGTNLEDGVPSRFSGSGSGTDFTLTISSLQPEDFATYYCVHYAQFPYTFGQGTKVEIK

SEQ ID NO: 164 - protein, 114 aa, Artificial Sequence; identical to SEQ ID NO: 158
  Description of Artificial Sequence: Synthetic peptide

SEQ ID NO: 165 - protein, 107 aa, Artificial Sequence
  Description of Artificial Sequence: Synthetic peptide
DIQMTQSPSSLSASVGDRVTITCHASQDISSYIVWYQQKPGKAPKGLIYHGTNLEDGVPSRFSGSGSGTDFTLTISSLQPEDFATYYCVHYAQFPYTFGQGTKVEIK

SEQ ID NO: 166 - protein, 114 aa, Artificial Sequence; identical to SEQ ID NO: 142
  Description of Artificial Sequence: Synthetic peptide

SEQ ID NO: 167 - protein, 107 aa, Artificial Sequence
  Description of Artificial Sequence: Synthetic peptide
DIQMTQSPSSLSASVGDRVTITCHASQDISSYIVWYQQKPGKSFKGLIYHGTNLEDGVPSRFSGSGSGTDFTLTISSLQPEDFATYYCAHYAQFPYTFGQGTKVEIK

SEQ ID NO: 168 - protein, 114 aa, Artificial Sequence; identical to SEQ ID NO: 142
  Description of Artificial Sequence: Synthetic peptide

SEQ ID NO: 169 - protein, 107 aa, Artificial Sequence
  Description of Artificial Sequence: Synthetic peptide
DIQMTQSPSSLSASVGDRVTITCHASQDISSYIVWYQQKPGKSFKGLIYHGTNLEDGVPSRFSGSGSGTDFTLTISSLQPEDFATYYCVAYAQFPYTFGQGTKVEIK

SEQ ID NO: 170 - protein, 114 aa, Artificial Sequence; identical to SEQ ID NO: 142
  Description of Artificial Sequence: Synthetic peptide

SEQ ID NO: 171 - protein, 107 aa, Artificial Sequence
  Description of Artificial Sequence: Synthetic peptide
DIQMTQSPSSLSASVGDRVTITCHASQDISSYIVWYQQKPGKSFKGLIYHGTNLEDGVPSRFSGSGSGTDFTLTISSLQPEDFATYYCVHAAQFPYTFGQGTKVEIK

SEQ ID NO: 172 - protein, 114 aa, Artificial Sequence; identical to SEQ ID NO: 142
  Description of Artificial Sequence: Synthetic peptide

SEQ ID NO: 173 - protein, 107 aa, Artificial Sequence
  Description of Artificial Sequence: Synthetic peptide
DIQMTQSPSSLSASVGDRVTITCHASQDISSYIVWYQQKPGKSFKGLIYHGTNLEDGVPSRFSGSGSGTDFTLTISSLQPEDFATYYCVHYAAFPYTFGQGTKVEIK

SEQ ID NO: 174 - protein, 114 aa, Artificial Sequence; identical to SEQ ID NO: 142
  Description of Artificial Sequence: Synthetic peptide

SEQ ID NO: 175 - protein, 107 aa, Artificial Sequence
  Description of Artificial Sequence: Synthetic peptide
DIQMTQSPSSLSASVGDRVTITCHASQDISSYIVWYQQKPGKSFKGLIYHGTNLEDGVPSRFSGSGSGTDFTLTISSLQPEDFATYYCVHYAQAPYTFGQGTKVEIK

SEQ ID NO: 176 - protein, 114 aa, Artificial Sequence; identical to SEQ ID NO: 142
  Description of Artificial Sequence: Synthetic peptide

SEQ ID NO: 177 - protein, 107 aa, Artificial Sequence
  Description of Artificial Sequence: Synthetic peptide
DIQMTQSPSSLSASVGDRVTITCHASQDISSYIVWYQQKPGKSFKGLIYHGTNLEDGVPSRFSGSGSGTDFTLTISSLQPEDFATYYCVHYAQFAYTFGQGTKVEIK

SEQ ID NO: 178 - protein, 114 aa, Artificial Sequence; identical to SEQ ID NO: 142
  Description of Artificial Sequence: Synthetic peptide

SEQ ID NO: 179 - protein, 107 aa, Artificial Sequence
  Description of Artificial Sequence: Synthetic peptide
DIQMTQSPSSLSASVGDRVTITCHASQDISSYIVWYQQKPGKSFKGLIYHGTNLEDGVPSRFSGSGSGTDFTLTISSLQPEDFATYYCVHYAQFPATFGQGTKVEIK

SEQ ID NO: 180 - protein, 114 aa, Artificial Sequence
  Description of Artificial Sequence: Synthetic peptide
EVQLVQSGAEVKKPGASVKVSCKASGYAFTNYLIEWVRQAPGQGLEWIGVINPGSGDTYYSEKFKGRVTLTADTSTSTAYLELSSLRSEDTAVYYCARARLDYWGQGTLVTVSS

SEQ ID NO: 181 - protein, 107 aa, Artificial Sequence; identical to SEQ ID NO: 145
  Description of Artificial Sequence: Synthetic peptide

SEQ ID NO: 182 - protein, 114 aa, Artificial Sequence
  Description of Artificial Sequence: Synthetic peptide
EVQLVQSGAEVKKPGASVKVSCKASGYAFTNYLIEWVRQAPGQGLEWIGVINPGSGDTYYSEKFKGRVTLTADTSTSTAYLELSSLRSEDTAVYYCARDALDYWGQGTLVTVSS

SEQ ID NO: 183 - protein, 107 aa, Artificial Sequence; identical to SEQ ID NO: 145
  Description of Artificial Sequence: Synthetic peptide

SEQ ID NO: 184 - protein, 114 aa, Artificial Sequence
  Description of Artificial Sequence: Synthetic peptide
EVQLVQSGAEVKKPGASVKVSCKASGYAFTNYLIEWVRQAPGQGLEWIGVINPGSGDTYYSEKFKGRVTLTADTSTSTAYLELSSLRSEDTAVYYCARDRADYWGQGTLVTVSS

SEQ ID NO: 185 - protein, 107 aa, Artificial Sequence; identical to SEQ ID NO: 145
  Description of Artificial Sequence: Synthetic peptide

SEQ ID NO: 186 - protein, 113 aa, Artificial Sequence
  Description of Artificial Sequence: Synthetic peptide
EVQLVESGPGLVKPSETLSLTCTVSGFSLTDYGVLWIRQPPGKGLEWIGMIWSGGTTDYNAAFISRVTISVDTSKNQFSLKLSSVTAADTAVYYCVREEMDYWGQGTLVTVSS

SEQ ID NO: 187 - protein, 107 aa, Artificial Sequence
  Description of Artificial Sequence: Synthetic peptide
DIQMTQSPSSLSASVGDRVTITCRASQDISNFLNWYQQKPGKAPKLLIYYTSRLHSGVPSRFSGSGSGTDFTLTISSLQPEDFATYYCQQGNTLPWTFGQGTKVEIK

SEQ ID NO: 188 - protein, 113 aa, Artificial Sequence
  Description of Artificial Sequence: Synthetic peptide
EVQLVESGPGLVKPSETLSLTCTVSGFSLTDYGVLWIRQPPGKGLEWIGMIWSGGTTDYNAAFISRVTISKDTSKNQVSLKLSSVTAADTAVYYCVREEMDYWGQGTLVTVSS

SEQ ID NO: 189 - protein, 107 aa, Artificial Sequence; identical to SEQ ID NO: 187
  Description of Artificial Sequence: Synthetic peptide

SEQ ID NO: 190 - protein, 113 aa, Artificial Sequence
  Description of Artificial Sequence: Synthetic peptide
EVQLVESGPGLVKPSETLSLTCTVSGFSLTDYGVLWVRQPPGKGLEWLGMIWSGGTTDYNAAFISRLTISKDTSKNQVSLKLSSVTAADTAVYYCVREEMDYWGQGTLVTVSS

SEQ ID NO: 191 - protein, 107 aa, Artificial Sequence; identical to SEQ ID NO: 187
  Description of Artificial Sequence: Synthetic peptide

SEQ ID NO: 192 - protein, 5 aa, Artificial Sequence
  Description of Artificial Sequence: Synthetic peptide
  MOD_RES (1)..(1): Xaa is D or E
  MOD_RES (2)..(2): Xaa is S or A
XXYMS

SEQ ID NO: 193 - protein, 17 aa, Artificial Sequence
  Description of Artificial Sequence: Synthetic peptide
  MOD_RES (6)..(6): Xaa is N or S
  MOD_RES (7)..(7): Xaa is A or G
  MOD_RES (8)..(8): Xaa is D or S
  MOD_RES (9)..(9): Xaa is A or S
DMYPDXXXXSYNQKFRE

SEQ ID NO: 194 - protein, 8 aa, Artificial Sequence
  Description of Artificial Sequence: Synthetic peptide
  MOD_RES (5)..(5): Xaa is Y or A
  MOD_RES (6)..(6): Xaa is A or F
  MOD_RES (7)..(7): Xaa is S or A
  MOD_RES (8)..(8): Xaa is A or V
APRWXXXX

SEQ ID NO: 195 - protein, 9 aa, Artificial Sequence
  Description of Artificial Sequence: Synthetic peptide
  MOD_RES (2)..(2): Xaa is A or Q
  MOD_RES (3)..(3): Xaa is A or G
  MOD_RES (4)..(4): Xaa is A or H
  MOD_RES (5)..(5): Xaa is A or T
  MOD_RES (6)..(6): Xaa is A or L
  MOD_RES (7)..(8): Xaa is, independently, A or P
QXXXXXXXT

SEQ ID NO: 196 - protein, 17 aa, Artificial Sequence
  Description of Artificial Sequence: Synthetic peptide
  MOD_RES (9)..(9): Xaa is T, A or Q
VINPGSGDXYYSEKFKG

SEQ ID NO: 197 - protein, 7 aa, Artificial Sequence
  Description of Artificial Sequence: Synthetic peptide
  MOD_RES (7)..(7): Xaa is S, E, or Q
HGTNLEX

SEQ ID NO: 198 - protein, 9 aa, Artificial Sequence
  Description of Artificial Sequence: Synthetic peptide
  MOD_RES (1)..(1): Xaa is V or A
  MOD_RES (2)..(2): Xaa is H or A
  MOD_RES (9)..(9): Xaa is Y or A
XXYAQFPYX

SEQ ID NO: 199 - skipped

SEQ ID NO: 200 - protein, 451 aa, Artificial Sequence
  Description of Artificial Sequence: Synthetic peptide
EVQLVESGGGLVQPGGSLRLSCAASGFTFSNYTMNWVRQAPGKGLEWVSAISGSGGSTYYADSVKGRFTISRDNSKNTLYLQMNSLRAEDTAVYYCAKDRYSQVHYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 219 aa, Artificial Sequence
  Description of Artificial Sequence: Synthetic peptide
DIVMTQSPDSLPVTPGEPASISCRSSQSLLHSNGYNYLDWYLQKAGQSPQLLIYLGSNRASGVPDRFSGSGSGTDFTLKISRVEAEDVGVYYCQQYYNHPTTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 219 aa, Artificial Sequence
  Description of Artificial Sequence: Synthetic peptide
DIQMTQSPDSLPVTPGEPASISCRSSQSLLHSNGYNYLDWYLQKAGQSPQLLIYLGSNRASGVPDRFSGSGSGTDFTLKISRVEAEDVGVYYCQQYYNHPTTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 450 aa, Artificial Sequence
  Description of Artificial Sequence: Synthetic peptide
EVQLVESGGGLVHPGGSLRLSCAGSGFTFSSYAMHWVRQAPGKGLEWVSAIGTGGGTYYADSVMGRFTISRDNSKNTLYLQMNSLRAEDTAVYYCARYDNVMGLYWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 214 aa, Artificial Sequence
  Description of Artificial Sequence: Synthetic peptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - protein, 118 aa, Artificial Sequence
  Description of Artificial Sequence: Synthetic peptide
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSS

SEQ ID NO: 206 - protein, 107 aa, Artificial Sequence
  Description of Artificial Sequence: Synthetic peptide
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIK

SEQ ID NO: 207 - protein, 124 aa, Artificial Sequence
  Description of Artificial Sequence: Synthetic peptide
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDQSTADYYFYYGMDVWGQGTTVTVSS

SEQ ID NO: 208 - protein, 106 aa, Artificial Sequence
  Description of Artificial Sequence: Synthetic peptide
EIVVTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 209 - protein, 122 aa, Artificial Sequence
  Description of Artificial Sequence: Synthetic peptide
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 210 - protein, 107 aa, Artificial Sequence
  Description of Artificial Sequence: Synthetic peptide
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 211 - protein, 120 aa, Artificial Sequence
  Description of Artificial Sequence: Synthetic peptide
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 212 - protein, 111 aa, Artificial Sequence
  Description of Artificial Sequence: Synthetic peptide
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 213 - protein, 469 aa, Artificial Sequence [sequence omitted]

SEQ ID NO: 214 - protein, 233 aa, Artificial Sequence
  Description of Artificial Sequence: Synthetic peptide
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 119 aa, Artificial Sequence
  Description of Artificial Sequence: Synthetic peptide
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCANYYGSSLSMDYWGQGTSVTVSS

SEQ ID NO: 216 - protein, 108 aa, Artificial Sequence
  Description of Artificial Sequence: Synthetic peptide
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKR

SEQ ID NO: 217 - protein, 121 aa, Artificial Sequence
  Description of Artificial Sequence: Synthetic peptide
EVQLQQSGPELVKPGASVKISCKTSGYTFKDYTMHWVKQSHGKSLEWIGGIYPNNGGSTYNQNFKDKATLTVDKSSSTAYMEFRSLTSEDSAVYYCARMGYHGPHLDFDVWGAGTTVTVSP

SEQ ID NO: 218 - protein, 108 aa, Artificial Sequence
  Description of Artificial Sequence: Synthetic peptide
DIVMTQSHKFMSTSLGDRVSITCKASQDVGAAVAWYQQKPGQSPKLLIYWASTRHTGVPDRFTGGGSGTDFTLTISNVQSEDLTDYFCQQYINYPLTFGGGTKLEIKR

SEQ ID NO: 219 - protein, 119 aa, Artificial Sequence
  Description of Artificial Sequence: Synthetic peptide
QVQLVQSGAEVKKPGASVKVSCKASGYTFTSYVMHWVRQAPGQRLEWMGYINPYNDGTKYNEKFKGRVTITSDTSASTAYMELSSLRSEDTAVYYCANYYGSSLSMDYWGQGTLVTVSS

SEQ ID NO: 220 - protein, 108 aa, Artificial Sequence
  Description of Artificial Sequence: Synthetic peptide
DIQMTQSPSSLSASVGDRVTITCRASQDISNYLNWYQQKPGKAPKLLIYYTSRLHSGVPSRFSGSGSGTDYTLTISSLQPEDFATYYCQQGNTLPWTFGQGTKVEIKR

SEQ ID NO: 221 - protein, 108 aa, Artificial Sequence
  Description of Artificial Sequence: Synthetic peptide
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPWTFGQGTKVEIKR

SEQ ID NO: 222 - protein, 119 aa, Artificial Sequence
  Description of Artificial Sequence: Synthetic peptide
QVQLVQSGAEVKKPGASVKVSCKASGYTFTSYVMHWVRQAPGQRLEWIGYINPYNDGTKYNEKFKGRATITSDTSASTAYMELSSLRSEDTAVYYCANYYGSSLSMDYWGQGTLVTVSS

SEQ ID NO: 223 - protein, 119 aa, Artificial Sequence
  Description of Artificial Sequence: Synthetic peptide
QVQLVQSGAEVKKPGASVKVSCKASGYTFTSYVMHWVRQAPGQRLEWIGYINPYNDGTKYNEKFKGRATLTSDKSASTAYMELSSLRSEDTAVYYCANYYGSSLSMDYWGQGTLVTVSS

SEQ ID NO: 224 - protein, 121 aa, Artificial Sequence
  Description of Artificial Sequence: Synthetic peptide
QVQLVQSGAEVKKPGSSVKVSCKASGYTFKDYTMHWVRQAPGQGLEWMGGIYPNNGGSTYNQNFKDRVTITADKSTSTAYMELSSLRSEDTAVYYCARMGYHGPHLDFDVWGQGTTVTVSS

SEQ ID NO: 225 - protein, 108 aa, Artificial Sequence
  Description of Artificial Sequence: Synthetic peptide
DIQMTQSPSSLSASVGDRVTITCKASQDVGAAVAWYQQKPGKAPKLLIYWASTRHTGVPSRFSGSGSGTDFTLTISSLQPEDFATYYCQQYINYPLTFGGGTKVEIKR

SEQ ID NO: 226 - protein, 108 aa, Artificial Sequence
  Description of Artificial Sequence: Synthetic peptide
DIQMTQSPSSLSASVGDRVTITCKASQDVGAAVAWYQQKPGKAPKLLIYWASTRHTGVPDRFSGGGSGTDFTLTISSLQPEDFATYYCQQYINYPLTFGGGTKVEIKR

SEQ ID NO: 227 - protein, 121 aa, Artificial Sequence
  Description of Artificial Sequence: Synthetic peptide
QVQLVQSGAEVKKPGSSVKVSCKASGYTFKDYTMHWVRQAPGQGLEWIGGIYPNNGGSTYNQNFKDRVTLTADKSTSTAYMELSSLRSEDTAVYYCARMGYHGPHLDFDVWGQGTTVTVSS

SEQ ID NO: 228 - protein, 121 aa, Artificial Sequence
  Description of Artificial Sequence: Synthetic peptide
QVQLVQSGAEVKKPGSSVKVSCKASGYTFKDYTMHWVRQAPGQGLEWIGGIYPNNGGSTYNQNFKDRATLTVDKSTSTAYMELSSLRSEDTAVYYCARMGYHGPHLDFDVWGQGTTVTVSS

SEQ ID NO: 229 - protein, 25 aa, Artificial Sequence [sequence omitted]

SEQ ID NO: 230 - protein, 13 aa, Artificial Sequence
  Description of Artificial Sequence: Synthetic peptide
WVRQAPGKGLEWV

SEQ ID NO: 231 - protein, 32 aa, Artificial Sequence
  Description of Artificial Sequence: Synthetic peptide
RFTISADTSKNTAYLQMNSLRAEDTAVYYCAR

SEQ ID NO: 232 - protein, 11 aa, Artificial Sequence
  Description of Artificial Sequence: Synthetic peptide
WGQGTLVTVSA

SEQ ID NO: 233 - protein, 23 aa, Artificial Sequence
  Description of Artificial Sequence: Synthetic peptide
DIQMTQSPSSLSASVGDRVTITC

SEQ ID NO: 234 - protein, 15 aa, Artificial Sequence
  Description of Artificial Sequence: Synthetic peptide
WYQQKPGKAPKLLIY

SEQ ID NO: 235 - protein, 32 aa, Artificial Sequence
  Description of Artificial Sequence: Synthetic peptide
GVPSRFSGSGSGTDFTLTISSLQPEDFATYYC

SEQ ID NO: 236 - protein, 11 aa, Artificial Sequence
  Description of Artificial Sequence: Synthetic peptide
FGQGTKVEIKR

(19) United States
(12) Patent Application Publication
(10) Pub. No.: US 2019/0169304 A1
(43) Pub. Date: Jun. 6, 2019

(54) COMBINATION THERAPY COMPRISING OX40 BINDING AGONISTS AND TIGIT INHIBITORS

(71) Applicant: Genentech, Inc., South San Francisco, CA (US)

(72) Inventors: Jeong M. Kim, San Francisco, CA (US); Jane L. Grogan, San Francisco, CA (US)

(21) Appl. No.: 16/271,520

(22) Filed: Feb. 8, 2019

Related U.S. Application Data
(63) Continuation of application No. 16/019,065, filed on Jun. 26, 2018.
Application No. 16/019,065 is a continuation of application No. 14/927,110, filed on Oct. 29, 2015, now abandoned.
(60) Provisional application No. 62/076,152, filed on Nov. 6, 2014.

(51) Int. Cl. C07K 16/28; C07K 16/30

(52) U.S. Cl. CPC C07K 16/2878; C07K 16/28; C07K 16/2803; A61K 2039/507; C07K 2317/732; C07K 2317/75; C07K 16/30

(57) ABSTRACT

The present invention describes combination therapy comprising an OX40 binding agonist and an agent that decreases or inhibits TIGIT expression and/or TIGIT activity and methods for use thereof, including methods of treating conditions where enhanced immunogenicity is desired, such as increasing tumor immunogenicity for the treatment of cancer or chronic infection.

### SEQUENCE LISTING

The instant application contains a Sequence Listing which has been submitted electronically in ASCII format and is hereby incorporated by reference in its entirety. Said ASCII copy, created on Feb. 7, 2019, is named 50474-104004_Sequence_Listing_2.7.19_ST25 and is 201,238 bytes in size.

### FIELD OF THE INVENTION

The present invention relates to combination therapy comprising an OX40 binding agonist and an agent that decreases or inhibits TIGIT expression and/or TIGIT activity.

### BACKGROUND

The provision of two distinct signals to T cells is a widely accepted model for lymphocyte activation of resting T lymphocytes by antigen-presenting cells (APCs). This model further provides for the discrimination of self from non-self and immune tolerance. The primary signal, or antigen-specific signal, is transduced through the T-cell receptor (TCR) following recognition of foreign antigen peptide presented in the context of the major histocompatibility complex (MHC). The second signal, or co-stimulatory signal, is delivered to T cells by co-stimulatory molecules expressed on antigen-presenting cells (APCs) and induces T cells to promote clonal expansion, cytokine secretion, and effector function. In the absence of co-stimulation, T cells can become refractory to antigen stimulation, which results in a tolerogenic response to either foreign or endogenous antigens.

In the two-signal model, T cells receive both positive co-stimulatory and negative co-inhibitory signals. The regulation of such positive and negative signals is critical to maximize the host's protective immune responses, while maintaining immune tolerance and preventing autoimmunity. Negative signals seem necessary for induction of T-cell tolerance, while positive signals promote T-cell activation. Both co-stimulatory and co-inhibitory signals are provided to antigen-exposed T cells, and the interplay between co-stimulatory and co-inhibitory signals is essential to controlling the magnitude of an immune response. Further, the signals provided to the T cells change as an infection or immune provocation is cleared, worsens, or persists, and these changes affect the responding T cells and re-shape the immune response.

The mechanism of co-stimulation is of therapeutic interest because the manipulation of co-stimulatory signals has shown to provide a means to either enhance or terminate cell-based immune response. OX40 (also known as CD34, TNFRSF4, or ACT35 antigen), a member of the tumor necrosis factor receptor superfamily, can provide co-stimulatory signals to CD4+ and CD8+ T cells, leading to enhanced cell proliferation, survival, effector function, and migration. OX40 signaling also enhances memory T cell development and function. OX40 is not constitutively expressed on naïve T cells, but is induced after engagement of the T cell receptor (TCR). The ligand for OX40, OX40L, is predominantly expressed on antigen presenting cells. OX40 is highly expressed by activated CD4+ T cells, activated CD8+ T cells, memory T cells, and regulatory T (Treg) cells.

Combining OX40 signaling with other signaling pathways that are deregulated in tumor cells may further enhance treatment efficacy. Thus, there remains a need for such an optimal therapy for treating or delaying development of various cancers, immune related diseases, and T cell dysfunctional disorders.

### SUMMARY

The present invention relates to combination therapy comprising an OX40 binding agonist and an agent that decreases or inhibits TIGIT expression and/or activity.

In one aspect, the invention features a method for treating or delaying progression of cancer in an individual comprising administering to the individual an effective amount of an OX40 binding agonist and an agent that decreases or inhibits TIGIT expression and/or activity.

In another aspect, the invention features a method for reducing or inhibiting cancer relapse or cancer progression in an individual comprising administering to the individual an effective amount of an OX40 binding agonist and an agent that decreases or inhibits TIGIT expression and/or activity.

In another aspect, the invention features a method for treating or delaying progression of an immune related disease in an individual comprising administering to the individual an effective amount of an OX40 binding agonist and an agent that decreases or inhibits TIGIT expression and/or activity. In another aspect, the invention features a method for reducing or inhibiting progression of an immune related disease in an individual comprising administering to the individual an effective amount of an OX40 binding agonist and an agent that decreases or inhibits TIGIT expression and/or activity. In some embodiments of these aspects, the immune related disease is associated with a T cell dysfunctional disorder. In some embodiments, the T cell dysfunctional disorder is characterized by decreased responsiveness to antigenic stimulation. In some embodiments, the T cell dysfunctional disorder is characterized by T cell anergy or decreased ability to secrete cytokines, proliferate, or execute cytolytic activity. In some embodiments, the T cell dysfunctional disorder is characterized by T cell exhaustion. In some embodiments, the T cells are CD4+ and CD8+ T cells. In some embodiments, the immune related disease is selected from the group consisting of unresolved acute infection, chronic infection, and tumor immunity.

In another aspect, the invention features a method of increasing, enhancing, or stimulating an immune response or function in an individual comprising administering to the individual an effective amount of an OX40 binding agonist and an agent that decreases or inhibits TIGIT expression and/or activity.

In another aspect, the invention features a method of treating or delaying progression of cancer in an individual comprising administering to the individual an effective amount of an OX40 binding agonist and an agent that modulates CD226 expression and/or activity.

In another aspect, the invention features a method for reducing or inhibiting cancer relapse or cancer progression in an individual comprising administering to the individual an effective amount of an OX40 binding agonist and an agent that modulates CD226 expression and/or activity.

In another aspect, the invention features a method for treating or delaying progression of an immune related disease in an individual comprising administering to the individual an effective amount of an OX40 binding agonist and an agent that modulates CD226 expression and/or activity. In another aspect, the invention features a method for reducing or inhibiting progression of an immune related disease in an individual comprising administering to the individual an effective amount of an OX40 binding agonist and an agent that modulates CD226 expression and/or activity. In some embodiments of these aspects, the immune related disease is associated with a T cell dysfunctional disorder. In some embodiments, the T cell dysfunctional disorder is characterized by decreased responsiveness to antigenic stimulation. In some embodiments, the T cell dysfunctional disorder is characterized by T cell anergy or decreased ability to secrete cytokines, proliferate, or execute cytolytic activity. In some embodiments, the T cell dysfunctional disorder is characterized by T cell exhaustion. In some embodiments, the T cell is a CD4+ T cell and/or a CD8+ T cell. In some embodiments, the immune related disease is selected from the group consisting of unresolved acute infection, chronic infection, and tumor immunity.

In another aspect, the invention features a method of increasing, enhancing, or stimulating an immune response or function in an individual comprising administering to the individual an effective amount of an OX40 binding agonist and an agent that modulates CD226 expression and/or activity.

In some embodiments, the agent that modulates CD226 expression and/or activity is an agent that increases and/or stimulates CD226 expression and/or activity. In some embodiments, the agent that modulates CD226 expression and/or activity is an agent that increases and/or stimulates the interaction of CD226 with PVR. In some embodiments, the agent that modulates CD226 expression and/or activity is an agent that increases and/or stimulates the intracellular signaling mediated by CD226 binding to PVR. In some embodiments, the agent that modulates CD226 expression and/or activity is selected from the group consisting of an agent that inhibits and/or blocks the interaction of CD226 with TIGIT, an antagonist of TIGIT expression and/or activity, an antagonist of PVR expression and/or activity, an agent that inhibits and/or blocks the interaction of TIGIT with PVR, an agent that inhibits and/or blocks the interaction of TIGIT with PVRL2, an agent that inhibits and/or blocks the interaction of TIGIT with PVRL3, an agent that inhibits and/or blocks the intracellular signaling mediated by TIGIT binding to PVR, an agent that inhibits and/or blocks the intracellular signaling mediated by TIGIT binding to PVRL2, an agent that inhibits and/or blocks the intracellular signaling mediated by TIGIT binding to PVRL3, and combinations thereof. In some embodiments, the agent that modulates CD226 expression and/or activity is an agent that inhibits and/or blocks the interaction of CD226 with TIGIT. In some embodiments, the agent that inhibits and/or blocks the interaction of CD226 with TIGIT is a small molecule inhibitor, an inhibitory antibody or antigen-binding fragment thereof, an aptamer, an inhibitory nucleic acid, or an inhibitory polypeptide. In some embodiments, the agent that inhibits and/or blocks the interaction of CD226 with TIGIT is an anti-TIGIT antibody or antigen-binding fragment thereof. In some embodiments, the agent that inhibits and/or blocks the interaction of CD226 with TIGIT is an inhibitory nucleic acid selected from the group consisting of an antisense polynucleotide, an interfering RNA, a catalytic RNA, and an RNA-DNA chimera. In some embodiments, the agent that modulates CD226 expression and/or activity is an antagonist of TIGIT expression and/or activity. In some embodiments, the antagonist of TIGIT expression and/or activity is a small molecule inhibitor, an inhibitory antibody or antigen-binding fragment thereof, an aptamer, an inhibitory nucleic acid, and an inhibitory polypeptide. In some embodiments, the antagonist of TIGIT expression and/or activity is an anti-TIGIT antibody or antigen-binding fragment thereof. In some embodiments, the antagonist of TIGIT expression and/or activity is an inhibitory nucleic acid selected from the group consisting of an antisense polynucleotide, an interfering RNA, a catalytic RNA, and an RNA-DNA chimera. In some embodiments, the antagonist of PVR expression and/or activity is selected from the group consisting of a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of TIGIT with PVR is selected from the group consisting of a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of TIGIT with PVRL2 is selected from the group consisting of a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of TIGIT with PVRL3 is selected from the group consisting of a small molecule inhibitor, an inhibitory antibody or antigen-binding fragment thereof, an aptamer, an inhibitory nucleic acid, and an inhibitory polypeptide. In some embodiments, the agent that inhibits and/or blocks the intracellular signaling mediated by TIGIT binding to PVR is selected from the group consisting of a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of TIGIT with PVRL2 is selected from the group consisting of a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of TIGIT with PVRL3 is selected from the group consisting of a small molecule inhibitor, an inhibitory antibody or antigen-binding fragment thereof, an aptamer, an inhibitory nucleic acid, and an inhibitory polypeptide.

In another aspect, the invention features a method of increasing, enhancing, or stimulating an immune response or function in an individual comprising administering to the individual an effective amount of an OX40 binding agonist, an effective amount of an agent that decreases or inhibits TIGIT expression and/or activity, and an agent that decreases or inhibits one or more additional immune co-inhibitory receptors. In some embodiments, the one or more additional immune co-inhibitory receptor is selected from the group consisting of PD-L1, PD-1, CTLA-4, LAG3, TIM3, BTLA, VISTA, B7H4, and CD96. In some embodiments, the one or more additional immune co-inhibitory receptor is selected from the group consisting of PD-L1, PD-1, CTLA-4, LAG3, and TIM3.

In another aspect, the invention features a method of increasing, enhancing, or stimulating an immune response or function in an individual comprising administering to the individual an effective amount of an OX40 binding agonist, an effective amount of an agent that decreases or inhibits TIGIT expression and/or activity, and an agent that increases or activates one or more additional immune co-stimulatory receptors or their ligands. In some embodiments, the one or more additional immune co-stimulatory receptors or their ligands is selected from the group consisting of CD226, CD28, CD27, CD137, HVEM, GITR, MICA, ICOS, NKG2D, and 2B4. In some embodiments, the one or more additional immune co-stimulatory receptors or their ligands is selected from the group consisting of CD226, CD27, CD137, HVEM, and GITR. In some embodiments, the one or more additional immune co-stimulatory receptors or their ligands is CD27.

In some embodiments of any one of the above aspects, the method further comprises administering at least one chemotherapeutic agent. In some embodiments, the individual has cancer. In some embodiments, the CD4 and/or CD8 T cells in the individual have increased or enhanced priming, activation, proliferation, cytokine release, and/or cytolytic activity relative to prior to the administration of the combination. In some embodiments, the number of CD4 and/or CD8 T cells is elevated relative to prior to administration of the combination. In some embodiments, the number of activated CD4 and/or CD8 T cells is elevated relative to prior to administration of the combination. In some embodiments, the activated CD4 and/or CD8 T cells are characterized by IFN-γ⁺ producing CD4 and/or CD8 T cells and/or enhanced cytolytic activity relative to prior to the administration of the combination. In some embodiments, the CD4 and/or CD8 T cells exhibit increased release of cytokines selected from the group consisting of IFN-γ, TNF-α, and interleukins. In some embodiments, the CD4 and/or CD8 T cells are effector memory T cells. In some embodiments, the CD4 and/or CD8 effector memory T cells are characterized by γ-IFN⁺ producing CD4 and/or CD8 T cells and/or enhanced cytolytic activity. In some embodiments, the CD4 and/or CD8 effector memory T cells are characterized by having the expression of CD44^{high} CD62L^{low}.

In some embodiments, the cancer has elevated levels of T cell infiltration. In some embodiments, the agent that decreases or inhibits TIGIT expression and/or activity is selected from the group consisting of an antagonist of TIGIT expression and/or activity, an antagonist of PVR expression and/or activity, an agent that inhibits and/or blocks the interaction of TIGIT with PVR, an agent that inhibits and/or blocks the interaction of TIGIT with PVRL2, an agent that inhibits and/or blocks the interaction of TIGIT with PVRL3, an agent that inhibits and/or blocks the intracellular signaling mediated by TIGIT binding to PVR, an agent that inhibits and/or blocks the intracellular signaling mediated by TIGIT binding to PVRL2, an agent that inhibits and/or blocks the intracellular signaling mediated by TIGIT binding to PVRL3, and combinations thereof. In some embodiments, the antagonist of TIGIT expression and/or activity is selected from the group consisting of a small molecule inhibitor, an inhibitory antibody or antigen-binding fragment thereof, an aptamer, an inhibitory nucleic acid, and an inhibitory polypeptide. In some embodiments, the antagonist of PVR expression and/or activity is selected from the group consisting of a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of TIGIT with PVR is selected from the group consisting of a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of TIGIT with PVRL2 is selected from the group consisting of a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of TIGIT with PVRL3 is selected from the group consisting of a small molecule inhibitor, an inhibitory antibody or antigen-binding fragment thereof, an aptamer, an inhibitory nucleic acid, and an inhibitory polypeptide. In some embodiments, the agent that inhibits and/or blocks the intracellular signaling mediated by TIGIT binding to PVR is selected from the group consisting of a small molecule inhibitor, an inhibitory antibody or antigen-binding fragment thereof, an aptamer, an inhibitory nucleic acid, and an inhibitory polypeptide. In some embodiments, the agent that inhibits and/or blocks the intracellular signaling mediated by TIGIT binding to PVRL2 is selected from the group consisting of a small molecule inhibitor, an inhibitory antibody or antigen-binding fragment thereof, an aptamer, an inhibitory nucleic acid, and an inhibitory polypeptide. In some embodiments, the agent that inhibits and/or blocks the intracellular signaling mediated by TIGIT binding to PVRL3 is selected from the group consisting of a small molecule inhibitor, an inhibitory antibody or antigen-binding fragment thereof, an aptamer, an inhibitory nucleic acid, and an inhibitory polypeptide. In some embodiments, the antagonist of TIGIT expression and/or activity is an inhibitory nucleic acid selected from the group consisting of an antisense polynucleotide, an interfering RNA, a catalytic RNA, and an RNA-DNA chimera. In some embodiments, the antagonist of TIGIT expression and/or activity is an anti-TIGIT antibody, or antigen-binding fragment thereof. In some embodiments, the anti-TIGIT antibody, or antigen-binding fragment thereof, comprises at least one HVR comprising an amino acid sequence selected from the amino acid sequences: (a) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (b) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12). In some embodiments, the anti-TIGIT antibody, or antigen-binding fragment thereof, comprises one of the following sets of six HVR sequences: (a) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (b) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12). In some embodiments, the anti-TIGIT antibody, or antigen-binding fragment thereof, comprises a light chain comprising the amino acid sequence set forth in DIVMTQSPSSLAVSPGEKVTMTCKSSQSLYYSGVKENLLAWYQQKPGQSP KLLIYYASIRFTGVPDRFTGSGSGTDYTLTITSVQAEDMGQYFCQQGINNPLTFGDGTKLEIKR (SEQ ID NO:13) or DVVLTQTPLSLSVSFGDQVSISCRSSQSLVNSYGNTFLSWYLHKPGQSPQLLIFGIS NRFSGVPDRFSGSGSGTDFTLKISTIKPEDLGMYYCLQGTHQPPTFGPGTKLEVK (SEQ ID NO:14). In some embodiments, the anti-TIGIT antibody, or antigen-binding fragment thereof, comprises a heavy chain comprising the amino acid sequence set forth in EVQLVESGGGLTQPGKSLKLSC EASGFTFSSFTMHWVRQSPGKGLEWVAFIRSGSGIVFYADAVRGRFTISRDNAKNLLFLQMNDLKSEDT AMYYCARRPLGHNTFDSWGQGTLVTVSS (SEQ ID NO:15) or EVQLQQSGPELVKPGTSMKIS CKASGYSFTGHLMNWVKQSHGKNLEWIGLIIPYNGGTSYNQKFKGKATLTVDKSSSTAYMELLSLTSDDS AVYFCSRGLRGFYAMDYWGQGTSVTVSS (SEQ ID NO:16). In some embodiments, the anti-TIGIT antibody, or antigen-binding fragment thereof, comprises a light chain comprising the amino acid sequence set forth in DIVMTQSPSSLAVSPGEKVTMTCKSSQSLYYSGVKENLLAWYQQKP GQSPKLLIYYASIRFTGVPDRFTGSGSGTDYTLTITSVQAEDMGQYFCQQGINNPLTFGDGTKLEIKR (SEQ ID NO:13) or DVVLTQTPLSLSVSFGDQVSISCRSSQSLVNSYGNTFLSWYLHKP GQSPQLLIFGISNRFSGVPDRFSGSGSGTDFTLKISTIKPEDLGMYYCLQGTHQPPTFGPGTKLEVK (SEQ ID NO:14), and a heavy chain comprising the amino acid sequence set forth in EVQLVESGGGLTQPGKSLKLSCEASGFTFSSFTMHWVRQSPGKGLEWVAFIRSGSGIVFYADAVRGRFT ISRDNAKNLLFLQMNDLKSEDTAMYYCARRPLGHNTFDSWGQGTLVTVSS (SEQ ID NO:15) or EVQLQQSGPELVKPGTSMKISCKASGYSFTGHLMNWVKQSHGKNLEWIGLIIPYNGGTSYNQKFKGKAT LTVDKSSSTAYMELLSLTSDDSAVYFCSRGLRGFYAMDYWGQGTSVTVSS (SEQ ID NO: 16). In some embodiments, the anti-TIGIT antibody, or antigen-binding fragment thereof, wherein the antibody is selected from the group consisting of a humanized antibody, a chimeric antibody, a bispecific antibody, a heteroconjugate antibody, and an immunotoxin. In some embodiments, the anti-TIGIT antibody, or antigen-binding fragment thereof, comprises at least one HVR that is at least 90% identical to an HVR set forth in any one of KSSQSLYYSGVKENLLA (SEQ ID NO: 1); ASIRFT (SEQ ID NO: 2); QQGINNPLT (SEQ ID NO: 3); GFTFSSFTMH (SEQ ID NO: 4); FIRSGSGIVFYADAVRG (SEQ ID NO: 5); RPLGHNTFDS (SEQ ID NO: 6); RSSQSLVNSYGNTFLS (SEQ ID NO: 7); GISNRFS (SEQ ID NO: 8); LQGTHQPPT (SEQ ID NO: 9); GYSFTGHLMN (SEQ ID NO: 10); LIIPYNGGTSYNQKFKG (SEQ ID NO: 11); and GLRGFYAMDY (SEQ ID NO: 12). In some embodiments, the anti-TIGIT antibody, or antigen-binding fragment thereof, comprises a light chain comprising amino acid sequences at least 90% identical to the amino acid sequences set forth in DIVMTQSPSSLAVSPGEKVTMTCKSSQSLYYSGVKENLLAWYQQKPGQSPKLLIYYASIRFTGVPDRFTG SGSGTDYTLTITSVQAEDMGQYFCQQGINNPLTFGDGTKLEIKR (SEQ ID NO:13) or DVVLTQTPLSLSVSFGDQVSISCRSSQSLVNSYGNTFLSWYLHKPGQSPQLLIFGISNRFSGVPDRFSGS GSGTDFTLKISTIKPEDLGMYYCLQGTHQPPTFGPGTKLEVK (SEQ ID NO:14); and/or comprises a heavy chain comprising amino acid sequences at least 90% identical to the amino acid sequences set forth in EVQLVESGGGLTQPGKSLKLSCEASGFTFSSFTMHWVRQSPGKGLEWVAFIRSGSGIVF YADAVRGRFTISRDNAKNLLFLQMNDLKSEDTAMYYCARRPLGHNTFDSWGQGTLVTVSS (SEQ ID NO:15) or EVQLQQSGPELVKPGTSMKISCKASGYSFTGHLMNWVKQSHGKNLEWIGLIIPYNGGTS YNQKFKGKATLTVDKSSSTAYMELLSLTSDDSAVYFCSRGLRGFYAMDYWGQGTSVTVSS (SEQ ID NO:16). In some embodiments, the anti-TIGIT antibody, or antigen-binding fragment thereof, binds to the same epitope as an antibody comprising one of the following sets of six HVR sequences: (a) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (b) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12).

In some embodiments of any one of above aspects, the OX40 binding agonist is selected from the group consisting of an OX40 agonist antibody, an OX40L agonist fragment, an OX40 oligomeric receptor, and an OX40 immunoadhesin. In some embodiments, the OX40 agonist antibody depletes cells that express human OX40. In some embodiments, the cells that express human OX40 are CD4+ effector T cells. In some embodiments, the cells that express human OX40 are regulatory T (Treg) cells. In some embodiments, the depleting is by ADCC and/or phagocytosis. In some embodiments, the depleting is by ADCC. In some embodiments, the OX40 agonist antibody binds human OX40 with an affinity of less than or equal to about 0.45 nM. In some embodiments, the OX40 agonist antibody binds human OX40 with an affinity of less than or equal to about 0.4 nM. In some embodiments, the binding affinity of the OX40 agonist antibody is determined using radioimmunoassay. In some embodiments, the OX40 agonist antibody binds human OX40 and cynomolgus OX40. In some embodiments, the binding is determined using a FACS assay. In some embodiments, the binding to human OX40 has an EC50 of less than or equal to 0.3 μg/ml. In some embodiments, the binding to human OX40 has an EC50 of less than or equal to 0.2 μg/ml. In some embodiments, the binding to cynomolgus OX40 has an EC50 of less than or equal to 1.5 μg/ml. In some embodiments, the binding to cynomolgus OX40 has an EC50 of less than or equal to 1.4 μg/ml. In some embodiments, the OX40 agonist antibody increases CD4+ effector T cell proliferation and/or increases cytokine production by the CD4+ effector T cell as compared to proliferation and/or cytokine (e.g., IFN-γ) production prior to treatment with the OX40 agonist antibody. In other embodiments, the OX40 agonist antibody increases memory T cell proliferation and/or increasing cytokine (e.g., IFN-γ) production by the memory cell. In some embodiments, the OX40 agonist antibody inhibits Treg function. In some embodiments, the OX40 agonist antibody inhibits Treg suppression of effector T cell function. In some embodiments, the effector T cell function is effector T cell proliferation and/or cytokine production. In some embodiments, the effector T cell is a CD4+ effector T cell. In some embodiments, the OX40 agonist antibody increases OX40 signal transduction in a target cell that expresses OX40. In some embodiments, the OX40 signal transduction is detected by monitoring NFkB downstream signaling. In some embodiments, the OX40 agonist antibody is stable after treatment at 40° C. for two weeks. In some embodiments, wherein the OX40 agonist antibody comprising a variant IgG1 Fc polypeptide comprising a mutation that eliminates binding to human effector cells has diminished activity relative to the OX40 agonist antibody comprising a native sequence IgG1 Fc portion. In some embodiments, the OX40 agonist antibody comprises a variant Fc portion comprising a DANA mutation. In some embodiments, antibody cross-linking is required for anti-human OX40 agonist antibody function.

In some embodiments of any one of the above aspects, the OX40 agonist antibody comprises (a) a VH domain comprising (i) HVR-H1 comprising the amino acid sequence of SEQ ID NO: 22, 28, or 29, (ii) HVR-H2 comprising the amino acid sequence of SEQ ID NO: 23, 30, 31, 32, 33 or 34, and (iii) HVR-H3 comprising an amino acid sequence selected from SEQ ID NO: 24, 35, or 39; and (iv) HVR-L1 comprising the amino acid sequence of SEQ ID NO: 25, (v) HVR-L2 comprising the amino acid sequence of SEQ ID NO: 26, and (vi) HVR-L3 comprising the amino acid sequence of SEQ ID NO: 27, 42, 43, 44, 45, 46, 47, or 48. In some embodiments, the OX40 agonist antibody comprises (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 25; (e) HVR-L2 comprising the amino acid sequence of SEQ ID NO: 26; and (f) HVR-L3 comprising an amino acid sequence selected from SEQ ID NO: 27. In some embodiments, the OX40 agonist antibody comprises (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 25; (e) HVR-L2 comprising the amino acid sequence of SEQ ID NO: 26; and (f) HVR-L3 comprising an amino acid sequence selected from SEQ ID NO: 46. In some embodiments, the OX40 agonist antibody comprises (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 25; (e) HVR-L2 comprising the amino acid sequence of SEQ ID NO: 26; and (f) HVR-L3 comprising an amino acid sequence selected from SEQ ID NO: 47. In some embodiments, the OX40 agonist antibody comprises a VH sequence having at least 90%, 91%, 92%, 93%, 94%, 95%, 96%, 97%, 98%, 99%, or 100% sequence identity to the amino acid sequence of SEQ ID NO: 76, 78, 80, 82, 84, 86, 88, 90, 92, 94, 96, 98, 100, 102, 104, 106, 108, 110, 112, 114, 116, 118, 120, 128, 134, or 136. In some embodiments, the OX40 agonist antibody comprises a VL having at least 90%, 91%, 92%, 93%, 94%, 95%, 96%, 97%, 98%, 99%, or 100% sequence identity to the amino acid sequence of SEQ ID NO: 77, 79, 81, 83, 85, 87, 89, 91, 93, 95, 97, 99, 101, 103, 105, 107, 109, 111, 113, 115, 117, 119, 121, 129, 135, or 137. In some embodiments, the OX40 agonist antibody comprises a VH sequence having at least 90%, 91%, 92%, 93%, 94%, 95%, 96%, 97%, 98%, 99%, or 100% sequence identity to the amino acid sequence of SEQ ID NO: 76. In some embodiments, the OX40 agonist antibody retains the ability to bind to human OX40. In some embodiments, a total of 1 to 10 amino acids have been substituted, inserted, and/or deleted in SEQ ID NO: 76. In some embodiments, the OX40 agonist antibody comprises a VH comprising one, two, or three HVRs selected from: (a) HVR-H1 comprising the amino acid sequence of SEQ ID NO: 22, (b) HVR-H2 comprising the amino acid sequence of SEQ ID NO: 23, and (c) HVR-H3 comprising the amino acid sequence of SEQ ID NO: 24. In some embodiments, the OX40 agonist antibody comprises a VL having at least 90%, 91%, 92%, 93%, 94%, 95%, 96%, 97%, 98%, 99%, or 100% sequence identity to the amino acid sequence of SEQ ID NO: 77. In some embodiments, the OX40 agonist antibody retains the ability to bind to human OX40. In some embodiments, a total of 1 to 10 amino acids have been substituted, inserted, and/or deleted in SEQ ID NO: 77. In some embodiments, the OX40 agonist antibody comprises a VL comprising one, two, or three HVRs selected from (a) HVR-L1 comprising the amino acid sequence of SEQ ID NO: 25; (b) HVR-L2 comprising the amino acid sequence of SEQ ID NO: 26; and (c) HVR-L3 comprising the amino acid sequence of SEQ ID NO: 27. In some embodiments, the OX40 agonist antibody comprises a VH sequence of SEQ ID NO: 76. In some embodiments, the OX40 agonist antibody comprises a VL sequence of SEQ ID NO: 77. In some embodiments, the OX40 agonist antibody comprises a VH sequence of SEQ ID NO: 76 and a VL sequence of SEQ ID NO: 77. In some embodiments, the OX40 agonist antibody comprises a VH sequence of SEQ ID NO: 114. In some embodiments, the OX40 agonist antibody comprises a VL sequence of SEQ ID NO: 115. In some embodiments, the OX40 agonist antibody comprises a VH sequence of SEQ ID NO: 114 and a VL sequence of SEQ ID NO: 115. In some embodiments, the OX40 agonist antibody comprises a VH sequence of SEQ ID NO: 116. In some embodiments, the OX40 agonist antibody comprises a VL sequence of SEQ ID NO: 117. In some embodiments, the OX40 agonist antibody comprises a VH sequence of SEQ ID NO: 116 and a VL sequence of SEQ ID NO: 117.

In some embodiments, the OX40 agonist antibody comprises (a) a heavy chain comprising an amino acid sequence having at least 90% sequence identity to the amino acid sequence of SEQ ID NO: 200; (b) a light chain comprising an amino acid sequence having at least 90% sequence identity to the amino acid sequence of SEQ ID NO: 201; or (c) both a heavy chain as in (a) and a light chain as in (b). In some embodiments, the OX40 agonist antibody comprises (a) a heavy chain comprising an amino acid sequence having at least 90% sequence identity to the amino acid sequence of SEQ ID NO: 203; (b) a light chain comprising an amino acid sequence having at least 90% sequence identity to the amino acid sequence of SEQ ID NO: 204; or (c) both a heavy chain as in (a) and a light chain as in (b). In some embodiments, the OX40 agonist antibody comprises (a) a VH comprising an amino acid sequence having at least 90% sequence identity to the amino acid sequence of SEQ ID NO: 205; (b) a VL comprising an amino acid sequence having at least 90% sequence identity to the amino acid sequence of SEQ ID NO: 206; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 207; (b) a VL comprising an amino acid sequence having at least 90% sequence identity to the amino acid sequence of SEQ ID NO: 208; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 209; (b) a VL comprising an amino acid sequence having at least 90% sequence identity to the amino acid sequence of SEQ ID NO: 210; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 211; (b) a VL comprising an amino acid sequence having at least 90% sequence identity to the amino acid sequence of SEQ ID NO: 212; or (c) both a VH as in (a) and a VL as in (b). In some embodiments, the OX40 agonist antibody comprises (a) a heavy chain comprising an amino acid sequence having at least 90% sequence identity to the amino acid sequence of SEQ ID NO: 213; (b) a light chain comprising an amino acid sequence having at least 90% sequence identity to the amino acid sequence of SEQ ID NO: 214; or (c) both a heavy chain as in (a) and a light chain as in (b). In some embodiments, the OX40 agonist antibody comprises (a) a VH comprising an amino acid sequence having at least 90% sequence identity to the amino acid sequence of SEQ ID NO: 215; (b) a VL comprising an amino acid sequence having at least 90% sequence identity to the amino acid sequence of SEQ ID NO: 216; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 217; (b) a VL comprising an amino acid sequence having at least 90% sequence identity to the amino acid sequence of SEQ ID NO: 218; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 219; (b) a VL comprising an amino acid sequence having at least 90% sequence identity to the amino acid sequence of SEQ ID NO: 220; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 219; (b) a VL comprising an amino acid sequence having at least 90% sequence identity to the amino acid sequence of SEQ ID NO: 221; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 222; (b) a VL comprising an amino acid sequence having at least 90% sequence identity to the amino acid sequence of SEQ ID NO: 220; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 222; (b) a VL comprising an amino acid sequence having at least 90% sequence identity to the amino acid sequence of SEQ ID NO: 221; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 223; (b) a VL comprising an amino acid sequence having at least 90% sequence identity to the amino acid sequence of SEQ ID NO: 220; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 223; (b) a VL comprising an amino acid sequence having at least 90% sequence identity to the amino acid sequence of SEQ ID NO: 221; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 224; (b) a VL comprising an amino acid sequence having at least 90% sequence identity to the amino acid sequence of SEQ ID NO: 225; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 224; (b) a VL comprising an amino acid sequence having at least 90% sequence identity to the amino acid sequence of SEQ ID NO: 226; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 227; (b) a VL comprising an amino acid sequence having at least 90% sequence identity to the amino acid sequence of SEQ ID NO: 225; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 227; (b) a VL comprising an amino acid sequence having at least 90% sequence identity to the amino acid sequence of SEQ ID NO: 226; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 228; (b) a VL comprising an amino acid sequence having at least 90% sequence identity to the amino acid sequence of SEQ ID NO: 225; or (c) both a VH as in (a) and a VL as in (b). In some embodiments, the OX40 agonist antibody comprises (a) a VH comprising an amino acid sequence having at least 90% sequence identity to the amino acid sequence of SEQ ID NO: 228; (b) a VL comprising an amino acid sequence having at least 90% sequence identity to the amino acid sequence of SEQ ID NO: 226; or (c) both a VH as in (a) and a VL as in (b). In some embodiments, the OX40 agonist antibody is antibody L106, antibody ACT35, MEDI6469, or MEDI0562. In some embodiments, the OX40 agonist antibody is a full-length IgG1 antibody. In some embodiments, the OX40 agonist antibody is an antibody fragment (e.g., an antigen-binding fragment). In some embodiments, the OX40 agonist antibody is selected from the group consisting of a humanized antibody, a chimeric antibody, a bispecific antibody, a heteroconjugate antibody, and an immunotoxin.

In other embodiments, the OX40 immunoadhesin is a trimeric OX40-Fc protein.

In some embodiments, the cancer is selected from the group consisting of non-small cell lung cancer, small cell lung cancer, renal cell cancer, colorectal cancer, ovarian cancer, breast cancer (e.g., triple-negative breast cancer), pancreatic cancer (e.g., pancreatic ductal adenocarcinoma (PDAC)), gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies.

In some embodiments, the agent that decreases or inhibits TIGIT expression and/or activity is administered continuously. In other embodiments, the agent that decreases or inhibits TIGIT expression and/or activity is administered intermittently. In some embodiments, the agent that decreases or inhibits TIGIT expression and/or activity is administered before the OX40 binding agonist. In other embodiments, the agent that decreases or inhibits TIGIT expression and/or activity is administered simultaneous with the OX40 binding agonist. In other embodiments, the agent that decreases or inhibits TIGIT expression and/or activity is administered after the OX40 binding agonist. In some embodiments, the OX40 binding agonist is administered before the agent that modulates CD226 expression and/or activity. In other embodiments, the OX40 binding agonist is administered simultaneous with the agent that modulates CD226 expression and/or activity. In other embodiments, the OX40 binding agonist is administered after the agent that modulates CD226 expression and/or activity. In some embodiments, the agent that decreases or inhibits TIGIT expression and/or activity is administered before the agent that decreases or inhibits one or more additional immune co-inhibitory receptors. In other embodiments, the agent that decreases or inhibits TIGIT expression and/or activity is administered simultaneous with the agent that decreases or inhibits one or more additional immune co-inhibitory receptors. In other embodiments, the agent that decreases or inhibits TIGIT expression and/or activity is administered after the agent that decreases or inhibits one or more additional immune co-inhibitory receptors. In some embodiments, the agent that decreases or inhibits TIGIT expression and/or activity is administered before the agent that increases or activates one or more additional immune co-stimulatory receptors or their ligands. In other embodiments, the agent that decreases or inhibits TIGIT expression and/or activity is administered simultaneous with the agent that increases or activates one or more additional immune co-stimulatory receptors or their ligands. In some embodiments, the agent that decreases or inhibits TIGIT expression and/or activity is administered after the agent that increases or activates one or more additional immune co-stimulatory receptors or their ligands. In some embodiments, the OX40 binding agonist is administered before the agent that decreases or inhibits one or more additional immune co-inhibitory receptors. In some embodiments, the OX40 binding agonist is administered simultaneous with the agent that decreases or inhibits one or more additional immune co-inhibitory receptors. In other embodiments, the OX40 binding agonist is administered after the agent that decreases or inhibits one or more additional immune co-inhibitory receptors. In some embodiments, the OX40 binding agonist is administered before the agent that increases or activates one or more additional immune co-stimulatory receptors or their ligands. In other embodiments, the OX40 binding agonist is administered simultaneous with the agent that increases or activates one or more additional immune co-stimulatory receptors or their ligands. In other embodiments, the OX40 binding agonist is administered after the agent that increases or activates one or more additional immune co-stimulatory receptors or their ligands.

In another aspect, the invention features a kit comprising an OX40 binding agonist and a package insert comprising instructions for using the OX40 binding agonist in combination with an agent that decreases or inhibits TIGIT expression and/or activity to treat or delay progression of cancer in an individual.

In another aspect, the invention features a kit comprising an OX40 binding agonist and an agent that decreases or inhibits TIGIT expression and/or activity, and a package insert comprising instructions for using the OX40 binding agonist and the agent that decreases or inhibits TIGIT expression and/or activity to treat or delay progression of cancer in an individual.

In another aspect, the invention features a kit comprising an agent that decreases or inhibits TIGIT expression and/or activity and a package insert comprising instructions for using the agent that decreases or inhibits TIGIT expression and/or activity in combination with an OX40 binding agonist to treat or delay progression of cancer in an individual.

In another aspect, the invention features a kit comprising an OX40 binding agonist and a package insert comprising instructions for using the OX40 binding agonist in combination with an agent that decreases or inhibits TIGIT expression and/or activity to enhance immune function of an individual having cancer.

In another aspect, the invention features a kit comprising an OX40 binding agonist and an agent that decreases or inhibits TIGIT expression and/or activity, and a package insert comprising instructions for using the OX40 binding agonist and the agent that decreases or inhibits TIGIT expression and/or activity to enhance immune function of an individual having cancer.

In another aspect, the invention features a kit comprising an agent that decreases or inhibits TIGIT expression and/or activity and a package insert comprising instructions for using the agent that decreases or inhibits TIGIT expression and/or activity in combination with an OX40 binding agonist to enhance immune function of an individual having cancer.

In another aspect, the invention features a kit comprising an OX40 binding agonist and a package insert comprising instructions for using the OX40 binding agonist in combination with an agent that modulates CD226 expression and/or activity to treat or delay progression of cancer in an individual.

In another aspect, the invention features a kit comprising an OX40 binding agonist and an agent that modulates CD226 expression and/or activity, and a package insert comprising instructions for using the OX40 binding agonist and the agent that modulates CD226 expression and/or activity to treat or delay progression of cancer in an individual.

In another aspect, the invention features a kit comprising an agent that modulates CD226 expression and/or activity and a package insert comprising instructions for using the agent modulates CD226 expression and/or activity in combination with an OX40 binding agonist to treat or delay progression of cancer in an individual.

In another aspect, the invention features a kit comprising an OX40 binding agonist and a package insert comprising instructions for using the OX40 binding agonist in combination with an agent that modulates CD226 expression and/or activity to enhance immune function of an individual having cancer.

In another aspect, the invention features a kit comprising an OX40 binding agonist and an agent that modulates CD226 expression and/or activity, and a package insert comprising instructions for using the OX40 binding agonist and the agent that modulates CD226 expression and/or activity to enhance immune function of an individual having cancer.

In another aspect, the invention features a kit comprising an agent modulates CD226 expression and/or activity and a package insert comprising instructions for using the agent that modulates CD226 expression and/or activity in combination with an OX40 binding agonist to enhance immune function of an individual having cancer.

### BRIEF DESCRIPTION OF THE DRAWINGS

FIGS. 1A and 1B are graphs showing that combination therapy of anti-OX40 agonist antibody and anti-TIGIT blocking antibody (clone 10A7) results in improved anti-tumor efficacy over either monotherapy in a syngeneic mice mouse tumor model, as depicted by mean tumor size (in mm³) linearly (FIG. 1A) or logarithmically (FIG. 1B) represented as a function of time (in days) following initial administration.

FIGS. 2A-2D are graphs showing the relative tumor sizes (in mm³) following initial administration of isotype control antibody (FIG. 2A), anti-OX40 agonist antibody (FIG. 2B), anti-TIGIT blocking antibody (clone 10A7) (FIG. 2C), or both anti-OX40 agonist antibody and anti-TIGIT blocking antibody (clone 10A7) (FIG. 2D) for each mouse within each arm of the study (n=10 mice per arm), linearly represented as a function of time (in days).

FIGS. 3A-3D are graphs showing the relative tumor sizes (in mm³) following initial administration of isotype control antibody (FIG. 3A), anti-OX40 agonist antibody (FIG. 3B), anti-TIGIT blocking antibody (clone 10A7) (FIG. 3C), or both anti-OX40 agonist antibody and anti-TIGIT blocking antibody (clone 10A7) (FIG. 3D) for each mouse within each arm of the study (n=10 mice per arm), logarithmically represented as a function of time (in days).

FIGS. 4A-4F are graphs showing the relative tumor sizes (in mm³) following initial administration of isotype control antibody (FIG. 4A), anti-OX40 agonist antibody at high (0.1 mg/kg) concentration (FIG. 4B), anti-OX40 agonist antibody at low (0.05 mg/kg) concentration (FIG. 4C), anti-TIGIT blocking antibody (clone 10A7) (FIG. 4D), both anti-OX40 agonist antibody at high (0.1 mg/kg) concentration and anti-TIGIT blocking antibody (clone 10A7) (FIG. 4E), and both anti-OX40 agonist antibody at low (0.05 mg/kg) concentration and anti-TIGIT blocking antibody (clone 10A7) (FIG. 4F) for each mouse within each arm of the study (n=10 mice per arm), linearly represented as a function of time (in days).

### DETAILED DESCRIPTION OF THE INVENTION

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calm, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

The term “OX40,” as used herein, refers to any native OX40 from any vertebrate source, including mammals such as primates (e.g., humans) and rodents (e.g., mice and rats), unless otherwise indicated. The term encompasses “full-length,” unprocessed OX40 as well as any form of OX40 that results from processing in the cell. The term also encompasses naturally occurring variants of OX40, for example, splice variants or allelic variants. The amino acid sequence of an exemplary human OX40 is shown in SEQ ID NO: 21.

“OX40 activation” refers to activation of the OX40 receptor. Generally, OX40 activation results in signal transduction.

The terms “anti-OX40 antibody” and “an antibody that binds to OX40” refer to an antibody that is capable of binding OX40 with sufficient affinity such that the antibody is useful as a diagnostic and/or therapeutic agent in targeting OX40. In one embodiment, the extent of binding of an anti-OX40 antibody to an unrelated, non-OX40 protein is less than about 10% of the binding of the antibody to OX40 as measured, e.g., by a radioimmunoassay (RIA). In certain embodiments, an antibody that binds to OX40 has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g. from 10⁻⁸ M to 10⁻¹³ M, e.g., from 10⁻⁹M to 10⁻¹³ M). In certain embodiments, an anti-OX40 antibody binds to an epitope of OX40 that is conserved among OX40 from different species.

The term “antagonist” is used in the broadest sense, and includes any molecule that partially or fully blocks, inhibits, or neutralizes a biological activity of a native polypeptide disclosed herein. In a similar manner, the term “agonist” is used in the broadest sense and includes any molecule that mimics a biological activity of a native polypeptide disclosed herein. Suitable agonist or antagonist molecules specifically include agonist or antagonist antibodies or antibody fragments, fragments or amino acid sequence variants of native polypeptides, peptides, antisense oligonucleotides, small organic molecules, etc. Methods for identifying agonists or antagonists of a polypeptide may comprise contacting a polypeptide with a candidate agonist or antagonist molecule and measuring a detectable change in one or more biological activities normally associated with the polypeptide.

The term “TIGIT” or “T-cell immunoreceptor with Ig and ITIM domains)” as used herein refers to any native TIGIT from any vertebrate source, including mammals such as primates (e.g. humans) and rodents (e.g., mice and rats), unless otherwise indicated. TIGIT is also known in the art as DKFZp667A205, FLJ39873, V-set and immunoglobulin domain-containing protein 9, V-set and transmembrane domain-containing protein 3, VSIG9, VSTM3, and WUCAM. The term encompasses “full-length,” unprocessed TIGIT as well as any form of TIGIT that results from processing in the cell. The term also encompasses naturally occurring variants of TIGIT, e.g., splice variants or allelic variants. The amino acid sequence of an exemplary human TIGIT may be found under UniProt Accession Number Q495A1.

The terms “TIGIT antagonist” and “antagonist of TIGIT activity or TIGIT expression” are used interchangeably and refer to a compound that interferes with the normal functioning of TIGIT, either by decreasing transcription or translation of TIGIT-encoding nucleic acid, or by inhibiting or blocking TIGIT polypeptide activity, or both. Examples of TIGIT antagonists include, but are not limited to, antisense polynucleotides, interfering RNAs, catalytic RNAs, RNA-DNA chimeras, TIGIT-specific aptamers, anti-TIGIT antibodies, TIGIT-binding fragments of anti-TIGIT antibodies, TIGIT-binding small molecules, TIGIT-binding peptides, and other polypeptides that specifically bind TIGIT (including, but not limited to, TIGIT-binding fragments of one or more TIGIT ligands, optionally fused to one or more additional domains), such that the interaction between the TIGIT antagonist and TIGIT results in a reduction or cessation of TIGIT activity or expression. It will be understood by one of ordinary skill in the art that in some instances, a TIGIT antagonist may antagonize one TIGIT activity without affecting another TIGIT activity. For example, a desirable TIGIT antagonist for use in certain of the methods herein is a TIGIT antagonist that antagonizes TIGIT activity in response to one of PVR interaction, PVRL3 interaction, or PVRL2 interaction, e.g., without affecting or minimally affecting any of the other TIGIT interactions.

The terms “PVR antagonist” and “antagonist of PVR activity or PVR expression” are used interchangeably and refer to a compound that interferes with the normal functioning of PVR, either by decreasing transcription or translation of PVR-encoding nucleic acid, or by inhibiting or blocking PVR polypeptide activity, or both. Examples of PVR antagonists include, but are not limited to, antisense polynucleotides, interfering RNAs, catalytic RNAs, RNA-DNA chimeras, PVR-specific aptamers, anti-PVR antibodies, PVR-binding fragments of anti-PVR antibodies, PVR-binding small molecules, PVR-binding peptides, and other polypeptides that specifically bind PVR (including, but not limited to, PVR-binding fragments of one or more PVR ligands, optionally fused to one or more additional domains), such that the interaction between the PVR antagonist and PVR results in a reduction or cessation of PVR activity or expression. It will be understood by one of ordinary skill in the art that in some instances, a PVR antagonist may antagonize one PVR activity without affecting another PVR activity. For example, a desirable PVR antagonist for use in certain of the methods herein is a PVR antagonist that antagonizes PVR activity in response to TIGIT interaction without impacting the PVR-CD96 and/or PVR-CD226 interactions.

The term “aptamer” refers to a nucleic acid molecule that is capable of binding to a target molecule, such as a polypeptide. For example, an aptamer of the invention can specifically bind to a TIGIT polypeptide, or to a molecule in a signaling pathway that modulates the expression of TIGIT. The generation and therapeutic use of aptamers are well established in the art. See, for example, U.S. Pat. No. 5,475,096, and the therapeutic efficacy of MACUGEN® (Eyetech, New York) for treating age-related macular degeneration.

The term “dysfunction,” in the context of immune dysfunction, refers to a state of reduced immune responsiveness to antigenic stimulation.

The term “dysfunctional,” as used herein, also includes refractory or unresponsive to antigen recognition, specifically, impaired capacity to translate antigen recognition into downstream T-cell effector functions, such as proliferation, cytokine production (e.g., gamma interferon) and/or target cell killing.

“Antibody-dependent cell-mediated cytotoxicity” or “ADCC” refers to a form of cytotoxicity in which secreted immunoglobulin bound onto Fc receptors (FcRs) present on certain cytotoxic cells (e.g., NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998). An exemplary assay for assessing ADCC activity is provided in the examples herein.

The term “anergy” refers to the state of unresponsiveness to antigen stimulation resulting from incomplete or insufficient signals delivered through the T-cell receptor (e.g., increase in intracellular Ca²⁺ in the absence of ras-activation). T cell anergy can also result upon stimulation with antigen in the absence of co-stimulation, resulting in the cell becoming refractory to subsequent activation by the antigen even in the context of costimulation. The unresponsive state can often be overridden by the presence of interleukin-2 (IL-2). Anergic T-cells do not undergo clonal expansion and/or acquire effector functions.

“Enhancing T cell function” means to induce, cause or stimulate an effector or memory T cell to have a renewed, sustained or amplified biological function. Examples of enhancing T-cell function include: increased secretion of γ-interferon from CD8⁺ effector T cells, increased secretion of γ-interferon from CD4+ memory and/or effector T-cells, increased proliferation of CD4+ effector and/or memory T cells, increased proliferation of CD8+ effector T-cells, increased antigen responsiveness (e.g., clearance), relative to such levels before the intervention. In one embodiment, the level of enhancement is at least 50%, alternatively 60%, 70%, 80%, 90%, 100%, 120%, 150%, 200%. The manner of measuring this enhancement is known to one of ordinary skill in the art.

The term “exhaustion” refers to T cell exhaustion as a state of T cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cells. Exhaustion prevents optimal control of infection and tumors. Exhaustion can result from both extrinsic negative regulatory pathways (e.g., immunoregulatory cytokines) as well as cell intrinsic negative regulatory (costimulatory) pathways (PD-1, B7-H3, B7-H4, etc.).

“Enhancing T-cell function” means to induce, cause or stimulate a T-cell to have a sustained or amplified biological function, or renew or reactivate exhausted or inactive T-cells. Examples of enhancing T-cell function include: increased secretion of γ-interferon from CD8+ T-cells, increased proliferation, increased antigen responsiveness (e.g., viral, pathogen, or tumor clearance) relative to such levels before the intervention. In one embodiment, the level of enhancement is as least 50%, alternatively 60%, 70%, 80%, 90%, 100%, 120%, 150%, 200%. The manner of measuring this enhancement is known to one of ordinary skill in the art.

A “T cell dysfunctional disorder” is a disorder or condition of T-cells characterized by decreased responsiveness to antigenic stimulation. In a particular embodiment, a T-cell dysfunctional disorder is a disorder that is specifically associated with inappropriate decreased signaling through OX40 and/or OX40L. In another embodiment, a T-cell dysfunctional disorder is one in which T-cells are anergic or have decreased ability to secrete cytokines, proliferate, or execute cytolytic activity. In a specific aspect, the decreased responsiveness results in ineffective control of a pathogen or tumor expressing an immunogen. Examples of T cell dysfunctional disorders characterized by T-cell dysfunction include unresolved acute infection, chronic infection, and tumor immunity.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage, and tumor clearance.

“Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include but are not limited to treatment with an OX40 binding agonist (e.g., anti-OX40 agonist antibodies) and a TIGIT inhibitor (e.g., anti-TIGIT blocking antibodies).

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)₂, and Fv). The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ, and μ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgA2.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

A “blocking antibody” or an “antagonist antibody” is one that inhibits or reduces a biological activity of the antigen it binds. In some embodiments, blocking antibodies or antagonist antibodies substantially or completely inhibit the biological activity of the antigen. The anti-TIGIT antibodies of the invention may block signaling through PVR, PVRL2, and/or PVRL3 so as to restore a functional response by T-cells (e.g., proliferation, cytokine production, target cell killing) from a dysfunctional state to antigen stimulation.

An “agonist antibody” or “activating antibody” is one that enhances or initiates signaling by the antigen to which it binds. In some embodiments, agonist antibodies cause or activate signaling without the presence of the natural ligand. The OX40 agonist antibodies of the invention may increase memory T cell proliferation, increase cytokine production by memory T cells, inhibit Treg cell function, and/or inhibit Treg cell suppression of effector T cell function, such as effector T cell proliferation and/or cytokine production.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The term “naked antibody” refers to an antibody that is not conjugated to a cytotoxic moiety or radiolabel.

The terms “full-length antibody,” “intact antibody” or “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen-binding and/or the variable region of the intact antibody. Examples of antibody fragments include Fab, Fab′, F(ab′)2 and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies of the invention comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. PharmacoL, 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5^{th} Ed. Public Health Service, National Institutes of Health, Bethesda, Md. (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

A “VH subgroup III consensus framework” comprises the consensus sequence obtained from the amino acid sequences in variable heavy subgroup III of Kabat et al., supra. In one embodiment, the VH subgroup III consensus framework amino acid sequence comprises at least a portion or all of each of the following sequences: EVQLVESGGGLVQPGGSLRLSCAAS (HC-FR1) (SEQ ID NO: 229); WVRQAPGKGLEWV (HC-FR2) (SEQ ID NO: 230); RFTISADTSKNTAYLQMNSLRAEDTAVYYCAR (HC-FR3) (SEQ ID NO: 232); and WGQGTLVTVSA (HC-FR4) (SEQ ID NO: 232).

A “VL kappa I consensus framework” comprises the consensus sequence obtained from the amino acid sequences in variable light kappa subgroup I of Kabat et al., supra. In one embodiment, the VH subgroup I consensus framework amino acid sequence comprises at least a portion or all of each of the following sequences: DIQMTQSPSSLSASVGDRVTITC (LC-FR1) (SEQ ID NO: 233); WYQQKPGKAPKLLIY (LC-FR2) (SEQ ID NO: 234); GVPSRFSGSGSGTDFTLTISSLQPEDFATYYC (LC-FR3) (SEQ ID NO: 235); and FGQGTKVEIKR (LC-FR4) (SEQ ID NO: 236).

An “amino-acid modification” at a specified position, for example, of the Fc region, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2 (including IgG2A and IgG2B), IgG-3, or IgG-4 subtypes, IgA (including IgA-1 and IgA-2), IgE, IgD or IgM. The Ig fusions preferably include the substitution of a domain of a polypeptide or antibody described herein in the place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995. For example, useful immunoadhesins for combination therapy herein include polypeptides that comprise the extracellular or OX40 binding portions of OX40L or the extracellular or OX40L binding portions of OX40, fused to a constant domain of an immunoglobulin sequence, such as a OX40 ECD-Fc or a OX40L ECD-Fc. Immunoadhesin combinations of Ig Fc and ECD of cell surface receptors are sometimes termed soluble receptors.

A “fusion protein” and a “fusion polypeptide” refer to a polypeptide having two portions covalently linked together, where each of the portions is a polypeptide having a different property. The property may be a biological property, such as activity in vitro or in vivo. The property may also be simple chemical or physical property, such as binding to a target molecule, catalysis of a reaction, etc. The two portions may be linked directly by a single peptide bond or through a peptide linker but are in reading frame with each other.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG1, IgG2 (IgG2A, IgG2B), IgG3 and IgG4.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

“Human effector cells” refer to leukocytes that express one or more FcRs and perform effector functions. In certain embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, e.g., from blood.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, the term “reducing or inhibiting cancer relapse” means to reduce or inhibit tumor or cancer relapse or tumor or cancer progression.

As used herein, “cancer” and “cancerous” refer to or describe the physiological condition in mammals that is typically characterized by unregulated cell growth. Included in this definition are benign and malignant cancers as well as dormant tumors or micrometastatses. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

An “effective amount” is at least the minimum concentration required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (La, slow to some extent or desirably stop) cancer cell infiltration into peripheral organs; inhibit (La, slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during, or after administration of the other treatment modality to the individual.

As used herein, “subject” or “individual” is meant a mammal, including, but not limited to, a human or non-human mammal, such as a bovine, equine, canine, ovine, or feline. Preferably, the subject is a human. Patients are also subjects herein.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, ft-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At211, I131, I125, Y90, Re186, Re188, Sm153, Bi212, P32, Pb212 and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-1a, IL-2, IL-3, IL-4, IL-5, IL-6, IL-7, IL-8, IL-9, IL10, IL-11, IL-12, IL-13, IL-15, IL-17A-F, IL-18 to IL-29 (such as IL-23), IL-31, including PROLEUKIN® ft-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The phrase “pharmaceutically acceptable” indicates that the substance or composition must be compatible chemically and/or toxicologically, with the other ingredients comprising a formulation, and/or the mammal being treated therewith.

The term “about” as used herein refers to the usual error range for the respective value readily known to the skilled person in this technical field. Reference to “about” a value or parameter herein includes (and describes) embodiments that are directed to that value or parameter per se.

### III. Methods

In one aspect, provided herein is a method for treating or delaying progression of cancer in an individual comprising administering to the individual an effective amount of an OX40 binding agonist in combination with an agent that decreases or inhibits TIGIT expression and/or activity.

In another aspect, provided herein is a method for reducing or inhibiting cancer relapse or cancer progression in an individual comprising administering to the individual an effective amount of an OX40 binding agonist in combination with an agent that that decreases or inhibits TIGIT expression and/or activity. As disclosed herein, cancer relapse and/or cancer progression include, without limitation, cancer metastasis.

In another aspect, provided herein is a method for treating or delaying progression of an immune related disease in an individual comprising administering to the individual an effective amount of an OX40 binding agonist in combination with an agent that decreases or inhibits TIGIT expression and/or activity.

In another aspect, provided herein is a method for reducing or inhibiting progression of an immune related disease in an individual comprising administering to the individual an effective amount of an OX40 binding agonist in combination with an agent that that decreases or inhibits TIGIT expression and/or activity.

In some embodiments, the immune related disease is associated with T cell dysfunctional disorder. In some embodiments, the immune related disease is a viral infection. In certain embodiments, the viral infection is a chronic viral infection. In some embodiments, T cell dysfunctional disorder is characterized by decreased responsiveness to antigenic stimulation. In some embodiments, the T cell dysfunctional disorder is characterized by T cell anergy or decreased ability to secrete cytokines, proliferate or execute cytolytic activity. In some embodiments, the T cell dysfunctional disorder is characterized by T cell exhaustion. In some embodiments, the T cells are CD4+ and CD8+ T cells. In some embodiments, the T cell dysfunctional disorder includes unresolved acute infection, chronic infection and tumor immunity.

In another aspect, provided herein is a method for increasing, enhancing or stimulating an immune response or function in an individual comprising administering to the individual an effective amount of an OX40 binding agonist in combination with an agent that decreases or inhibits TIGIT expression and/or activity.

In another aspect, provided herein is a method of treating or delaying progression of cancer in an individual comprising administering to the individual an effective amount of an OX40 binding agonist and an agent that modulates the CD226 expression and/or activity.

In another aspect, provided herein is a method for reducing or inhibiting cancer relapse or cancer progression in an individual comprising administering to the individual an effective amount of an OX40 binding agonist and an agent that modulates the CD226 expression and/or activity.

In another aspect, provided herein is a method for treating or delaying progression of an immune related disease in an individual comprising administering to the individual an effective amount of an OX40 binding agonist and an agent that modulates the CD226 expression and/or activity.

In another aspect, provided herein is a method for reducing or inhibiting progression of an immune related disease in an individual comprising administering to the individual an effective amount of an OX40 binding agonist and agent that modulates the CD226 expression and/or activity.

In some embodiments, the immune related disease is associated with T cell dysfunctional disorder. In some embodiments, the immune related disease is a viral infection. In certain embodiments, the viral infection is a chronic viral infection. In some embodiments, the T cell dysfunctional disorder is characterized by decreased responsiveness to antigenic stimulation. In some embodiments, the T cell dysfunctional disorder is characterized by T cell anergy, or decreased ability to secrete cytokines, proliferate or execute cytolytic activity. In some embodiments, the T cell dysfunctional disorder is characterized by T cell exhaustion. In some embodiments, the T cells are CD4+ and CD8+ T cells. In some embodiments, the immune related disease is selected from the group consisting of unresolved acute infection, chronic infection and tumor immunity.

In another aspect, provided herein is a method of increasing, enhancing or stimulating an immune response or function in an individual by administering to the individual an effective amount of an OX40 binding agonist and an agent that modulates the CD226 expression and/or activity.

In some embodiments, the agent that modulates the CD226 expression and/or activity is capable of increasing and/or stimulating CD226 expression and/or activity; increasing and/or stimulating the interaction of CD226 with PVR, PVRL2, and/or PVRL3; and increasing and/or stimulating the intracellular signaling mediated by CD226 binding to PVR, PVRL2, and/or PVRL3. As used herein, an agent that is capable of increasing and/or stimulating CD226 expression and/or activity includes, without limitation, agents that increase and/or stimulate CD226 expression and/or activity. As used herein, an agent that is capable of increasing and/or stimulating the interaction of CD226 with PVR, PVRL2, and/or PVRL3 includes, without limitation, agents that increase and/or stimulate the interaction of CD226 with PVR, PVRL2, and/or PVRL3. As used herein, an agent that is capable of increasing and/or stimulating the intracellular signaling mediated by CD226 binding to PVR, PVRL2, and/or PVRL3 includes, without limitation, agents that increase and/or stimulate the intracellular signaling mediated by CD226 binding to PVR, PVRL2, and/or PVRL3.

In some embodiments, the agent that modulates the CD226 expression and/or activity is selected from an agent that inhibits and/or blocks the interaction of CD226 with TIGIT, an antagonist of TIGIT expression and/or activity, an antagonist of PVR expression and/or activity, an agent that inhibits and/or blocks the interaction of TIGIT with PVR, an agent that inhibits and/or blocks the interaction of TIGIT with PVRL2, an agent that inhibits and/or blocks the interaction of TIGIT with PVRL3, an agent that inhibits and/or blocks the intracellular signaling mediated by TIGIT binding to PVR, an agent that inhibits and/or blocks the intracellular signaling mediated by TIGIT binding to PVRL2, an agent that inhibits and/or blocks the intracellular signaling mediated by TIGIT binding to PVRL3, and combinations thereof.

In some embodiments, the agent that inhibits and/or blocks the interaction of CD226 with TIGIT is a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of CD226 with TIGIT is an anti-TIGIT antibody or antigen-binding fragment thereof. In some embodiments, the agent that inhibits and/or blocks the interaction of CD226 with TIGIT is an inhibitory nucleic acid selected from an antisense polynucleotide, an interfering RNA, a catalytic RNA, and an RNA-DNA chimera.

In some embodiments, the antagonist of TIGIT expression and/or activity is a small molecule inhibitor, an inhibitory antibody or antigen-binding fragment thereof, an aptamer, an inhibitory nucleic acid, and an inhibitory polypeptide. In some embodiments, the antagonist of TIGIT expression and/or activity is an anti-TIGIT antibody or antigen-binding fragment thereof. In some embodiments, the antagonist of TIGIT expression and/or activity is an inhibitory nucleic acid selected from an antisense polynucleotide, an interfering RNA, a catalytic RNA, and an RNA-DNA chimera.

In some embodiments, the antagonist of PVR expression and/or activity is a small molecule inhibitor, an inhibitory antibody or antigen-binding fragment thereof, an aptamer, an inhibitory nucleic acid, and an inhibitory polypeptide. In some embodiments, the antagonist of PVR expression and/or activity is selected from a small molecule inhibitor, an inhibitory antibody or antigen-binding fragment thereof, an aptamer, an inhibitory nucleic acid, and an inhibitory polypeptide.

In some embodiments, the agent that inhibits and/or blocks the interaction of TIGIT with PVR is a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of TIGIT with PVR is selected from a small molecule inhibitor, an inhibitory antibody or antigen-binding fragment thereof, an aptamer, an inhibitory nucleic acid, and an inhibitory polypeptide.

In some embodiments, the agent that inhibits and/or blocks the interaction of TIGIT with PVRL2 is selected from a small molecule inhibitor, an inhibitory antibody or antigen-binding fragment thereof, an aptamer, an inhibitory nucleic acid, and an inhibitory polypeptide.

In some embodiments, the agent that inhibits and/or blocks the interaction of TIGIT with PVRL3 is selected from a small molecule inhibitor, an inhibitory antibody or antigen-binding fragment thereof, an aptamer, an inhibitory nucleic acid, and an inhibitory polypeptide.

In some embodiments, the agent that inhibits and/or blocks the intracellular signaling mediated by TIGIT binding to PVR is a small molecule inhibitor, an inhibitory antibody or antigen-binding fragment thereof, an aptamer, an inhibitory nucleic acid, and an inhibitory polypeptide. In some embodiments, the agent that inhibits and/or blocks the intracellular signaling mediated by TIGIT binding to PVR is selected from a small molecule inhibitor, an inhibitory antibody or antigen-binding fragment thereof, an aptamer, an inhibitory nucleic acid, and an inhibitory polypeptide.

In some embodiments, the agent that inhibits and/or blocks the intracellular signaling mediated by TIGIT binding to PVRL2 is selected from a small molecule inhibitor, an inhibitory antibody or antigen-binding fragment thereof, an aptamer, an inhibitory nucleic acid, and an inhibitory polypeptide.

In some embodiments, the agent that inhibits and/or blocks the intracellular signaling mediated by TIGIT binding to PVRL3 is selected from a small molecule inhibitor, an inhibitory antibody or antigen-binding fragment thereof, an aptamer, an inhibitory nucleic acid, and an inhibitory polypeptide.

In another aspect, provided herein is a method of increasing, enhancing or stimulating an immune response or function in an individual by administering to the individual an effective amount of an agent that decreases or inhibits TIGIT expression and/or activity and an agent that decreases or inhibits the expression and/or activity of one or more additional immune co-inhibitory receptors. In some embodiments, the one of more additional immune co-inhibitory receptor is selected from PD-L1, PD-1, CTLA-4, LAG3, TIM3, BTLA VISTA, B7H4, and CD96. In some embodiments, one of more additional immune co-inhibitory receptor is selected from PD-L1, PD-1, CTLA-4, LAG3, and TIM3.

In another aspect, provided herein is a method of increasing, enhancing or stimulating an immune response or function in an individual by administering to the individual an effective amount of an agent that decreases or inhibits TIGIT expression and/or activity and an agent that increases or activates the expression and/or activity of one or more additional immune co-stimulatory receptors or their ligands. In some embodiments, the one of more additional immune co-stimulatory receptor or ligand is selected from CD226, CD28, CD27, CD137, HVEM, GITR, MICA, ICOS, NKG2D, and 2B4. In some embodiments, the one or more additional immune co-stimulatory receptor is selected from CD226, CD28, CD27, CD137, HVEM, and GITR. In some embodiments, the one of more additional immune co-stimulatory receptor is CD27.

The methods of this invention may find use in treating conditions where enhanced immunogenicity is desired such as increasing tumor immunogenicity for the treatment of cancer or T cell dysfunctional disorders.

A variety of cancers may be treated, or their progression may be delayed. In some embodiments, the individual may have breast cancer (e.g., triple-negative breast cancer). In other embodiments, the individual may have pancreatic cancer (e.g., pancreatic ductal adenocarcinoma (PDAC)).

In some embodiments, the individual has non-small cell lung cancer. The non-small cell lung cancer may be at early stage or at late stage. In some embodiments, the individual has small cell lung cancer. The small cell lung cancer may be at early stage or at late stage. In some embodiments, the individual has renal cell cancer. The renal cell cancer may be at early stage or at late stage. In some embodiments, the individual has colorectal cancer. The colorectal cancer may be at early stage or late stage. In some embodiments, the individual has ovarian cancer. The ovarian cancer may be at early stage or at late stage. In some embodiments, the individual has breast cancer. The breast cancer may be at early stage or at late stage. In some embodiments, the individual has pancreatic cancer. The pancreatic cancer may be at early stage or at late stage. In some embodiments, the individual has gastric carcinoma. The gastric carcinoma may be at early stage or at late stage. In some embodiments, the individual has bladder cancer. The bladder cancer may be at early stage or at late stage. In some embodiments, the individual has esophageal cancer. The esophageal cancer may be at early stage or at late stage. In some embodiments, the individual has mesothelioma. The mesothelioma may be at early stage or at late stage. In some embodiments, the individual has melanoma. The melanoma may be at early stage or at late stage. In some embodiments, the individual has head and neck cancer. The head and neck cancer may be at early stage or at late stage. In some embodiments, the individual has thyroid cancer. The thyroid cancer may be at early stage or at late stage. In some embodiments, the individual has sarcoma. The sarcoma may be at early stage or late stage. In some embodiments, the individual has prostate cancer. The prostate cancer may be at early stage or at late stage. In some embodiments, the individual has glioblastoma. The glioblastoma may be at early stage or at late stage. In some embodiments, the individual has cervical cancer. The cervical cancer may be at early stage or at late stage. In some embodiments, the individual has thymic carcinoma. The thymic carcinoma may be at early stage or at late stage. In some embodiments, the individual has leukemia. The leukemia may be at early stage or at late stage. In some embodiments, the individual has lymphomas. The lymphoma may be at early stage or at late stage. In some embodiments, the individual has myelomas. The myelomas may be at early stage or at late stage. In some embodiments, the individual has mycoses fungoids. The mycoses fungoids may be at early stage or at late stage. In some embodiments, the individual has merkel cell cancer. The merkel cell cancer may be at early stage or at late stage. In some embodiments, the individual has hematologic malignancies. The hematological malignancies may be early stage or late stage. In some embodiments, the individual is a human.

In some embodiments of the methods of this invention, the CD4 and/or CD8 T cells in the individual have increased or enhanced priming, activation, proliferation, cytokine release and/or cytolytic activity relative to prior to the administration of the combination.

In some embodiments of the methods of this invention, the number of CD4 and/or CD8 T cells is elevated relative to prior to administration of the combination. In some embodiments of the methods of this invention, the number of activated CD4 and/or CD8 T cells is elevated relative to prior to administration of the combination.

In some embodiments of the methods of this invention, the activated CD4 and/or CD8 T cells is characterized by γ-IFN⁺ producing CD4 and/or CD8 T cells and/or enhanced cytolytic activity relative to prior to the administration of the combination.

In some embodiments of the methods of this invention, the CD4 and/or CD8 T cells exhibit increased release of cytokines selected from the group consisting of IFN-γ, TNF-α and interleukins.

In some embodiments of the methods of this invention, the CD4 and/or CD8 T cell is an effector memory T cell. In some embodiments of the methods of this invention, the CD4 and/or CD8 effector memory T cell is characterized by γ-IFN⁺ producing CD4 and/or CD8 T cells and/or enhanced cytolytic activity. In some embodiments of the methods of this invention, the CD4 and/or CD8 effector memory T cell is characterized by having the expression of CD44^{high} CD62L^{low}.

In some embodiments of the methods of this invention, the cancer has elevated levels of T cell infiltration.

In some embodiments, the methods of the invention may further comprise administering an additional therapy. The additional therapy may be radiation therapy, surgery, chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of an adjuvant or neoadjuvant therapy. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy may be one or more of the chemotherapeutic agents described hereinabove.

Any of the OX40 binding agonists and agents that decreases or inhibits TIGIT expression and/or activity described below may be used in the methods of the invention.

In some embodiments, any of the targets described herein (e.g., PD-1, PD-L1, PD-L2, CTLA-4, LAGS, TIM3, BTLA, VISTA, B7H4, CD96, B7-1, TIGIT, CD226, OX40, CD28, CD27, CD137, HVEM, GITR, MICA, ICOS, NKG2D, 2B4, etc.) is a human protein.

A. OX40 Binding Agonists

Provided herein is a method for treatment or delaying progression of cancer in an individual comprising administering to the individual an effective amount of an OX40 binding agonist in combination with an agent that decreases or inhibits TIGIT expression and/or activity. Provided herein is also a method for reducing or inhibiting cancer relapse or cancer progression in an individual comprising administering to the individual an effective amount of an OX40 binding agonist in combination with an agent that that decreases or inhibits TIGIT expression and/or activity. Provided herein is also a method for treating or delaying progression of an immune related disease in an individual comprising administering to the individual an effective amount of an OX40 binding agonist in combination with an agent that that decreases or inhibits TIGIT expression and/or activity. Provided herein is also a method for reducing or inhibiting progression of an immune related disease in an individual comprising administering to the individual an effective amount of an OX40 binding agonist in combination with an agent that that decreases or inhibits TIGIT expression and/or activity. Provided herein is also a method for increasing, enhancing or stimulating an immune response or function in an individual comprising administering to the individual an effective amount of an OX40 binding agonist in combination with an agent that decreases or inhibits TIGIT expression and/or activity.

An OX40 binding agonist includes, for example, an OX40 agonist antibody (e.g., an anti-human OX40 agonist antibody), an OX40L agonist fragment, an OX40 oligomeric receptor, and an OX40 immunoadhesin.

In some embodiments, the OX40 agonist antibody depletes cells that express human OX40 (e.g., CD4+ effector T cells, CD8+ T cells, and/or Treg cells), for example, by ADCC and/or phagocytosis. In some embodiments, the OX40 agonist antibody binds human OX40 with an affinity of less than or equal to about 1 nM (e.g., less than or equal to about 0.5 nM, e.g., less than or equal to about 0.45 nM, e.g., less than or equal to about 0.4 nM, e.g., less than or equal to about 0.3 nM). In some embodiments, the binding affinity of the OX40 agonist antibody is determined using radioimmunoassay.

In some embodiments, the OX40 agonist antibody binds human OX40 and cynomolgus OX40. In further embodiments, binding to human OX40 and cynomolgus OX40 is determined using a FACS assay. In some embodiments, binding to human OX40 has an EC50 of less than or equal to about 1 μg/ml (e.g., less than or equal to about 0.7 μg/ml, e.g., less than or equal to about 0.5 μg/ml, e.g., less than or equal to about 0.4 μg/ml, e.g., less than or equal to about 0.3 μg/ml, e.g., less than or equal to about 0.2 μg/ml, e.g., less than or equal to about 0.1 μg/ml). In some embodiments, binding to cynomolgus OX40 has an EC50 of less than or equal to 3 μg/ml (e.g., less than or equal to about 2 μg/ml, e.g., less than or equal to about 1.7 μg/ml, e.g., less than or equal to about 1.5 μg/ml, e.g., less than or equal to about 1.4 μg/ml, e.g., less than or equal to about 1.3 μg/ml, e.g., less than or equal to about 1.2 μg/ml, e.g., less than or equal to about 1.1 μg/ml, e.g., less than or equal to about 1.0 μg/ml).

In some embodiments, the OX40 agonist antibody increases CD4+ effector T cell proliferation and/or increases cytokine production by the CD4+ effector T cell as compared to proliferation and/or cytokine production prior to treatment with the OX40 agonist antibody. In some embodiments, the cytokine is IFN-γ.

In some embodiments, the OX40 agonist antibody increases memory T cell proliferation and/or increasing cytokine production by the memory cell. In some embodiments, the cytokine is IFN-γ.

In some embodiments, the OX40 agonist antibody inhibits Treg suppression of effector T cell function. In some embodiments, effector T cell function is effector T cell proliferation and/or cytokine production. In some embodiments, the effector T cell is a CD4+ effector T cell.

In some embodiments, the OX40 agonist antibody increases OX40 signal transduction in a target cell that expresses OX40. In some embodiments, OX40 signal transduction is detected by monitoring NFkB downstream signaling.

In some embodiments, the OX40 agonist antibody is stable after treatment at 40° C. for one to four weeks, e.g., one week, two weeks, three weeks, or four weeks. In some embodiments, the OX40 agonist antibody is stable after treatment at 40° C. for two weeks.

In some embodiments, the OX40 agonist antibody comprises a variant IgG1 Fc polypeptide comprising a mutation that eliminates binding to human effector cells has diminished activity relative to the OX40 agonist antibody comprising a native sequence IgG1 Fc portion. In some embodiments, the OX40 agonist antibody comprises a variant Fc portion comprising a DANA mutation.

In some embodiments, antibody cross-linking is required for anti-human OX40 antagonist antibody function.

In some embodiments, the OX40 agonist antibody comprises (a) a VH domain comprising one, two, or three of the following: (i) HVR-H1 comprising the amino acid sequence of SEQ ID NO: 22, 28, or 29, (ii) HVR-H2 comprising the amino acid sequence of SEQ ID NO: 23, 30, 31, 32, 33 or 34, and (iii) HVR-H3 comprising an amino acid sequence selected from SEQ ID NO: 24, 35, or 39; and/or one, two, or three of the following: (iv) HVR-L1 comprising the amino acid sequence of SEQ ID NO: 25, (v) HVR-L2 comprising the amino acid sequence of SEQ ID NO: 26, and (vi) HVR-L3 comprising the amino acid sequence of SEQ ID NO: 27, 42, 43, 44, 45, 46, 47, or 48. In certain embodiments, the OX40 agonist antibody comprises (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 25; (e) HVR-L2 comprising the amino acid sequence of SEQ ID NO: 26; and (f) HVR-L3 comprising an amino acid sequence selected from SEQ ID NO: 27. In other embodiments, the OX40 agonist antibody comprises (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 25; (e) HVR-L2 comprising the amino acid sequence of SEQ ID NO: 26; and (f) HVR-L3 comprising an amino acid sequence selected from SEQ ID NO: 46. In another embodiment, the OX40 agonist antibody comprises (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 25; (e) HVR-L2 comprising the amino acid sequence of SEQ ID NO: 26; and (f) HVR-L3 comprising an amino acid sequence selected from SEQ ID NO: 47.

In some embodiments, the OX40 agonist antibody comprises a VH sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to, or the sequence of, SEQ ID NO: 76, 78, 80, 82, 84, 86, 88, 90, 92, 94, 96, 98, 100, 102, 104, 106, 108, 110, 112, 114, 116, 118, 120, 128, 134, or 136.

In some embodiments, the OX40 agonist antibody comprises a VL having at least 80%, 81%, 82%, 83%, 84%, 85%, 86%, 87%, 88%, 89%, 90%, 91%, 92%, 93%, 94%, 95%, 96%, 97%, 98%, or 99% sequence identity to, or the sequence of, SEQ ID NO: 77, 79, 81, 83, 85, 87, 89, 91, 93, 95, 97, 99, 101, 103, 105, 107, 109, 111, 113, 115, 117, 119, 121, 129, 135, or 137.

In some embodiments, the OX40 agonist antibody comprises a VH sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to, or the sequence of, SEQ ID NO: 76. In certain embodiments, the OX40 agonist antibody retains the ability to bind to human OX40. In some embodiments, a total of 1 to 20 amino acids have been substituted, inserted, and/or deleted in SEQ ID NO: 76, e.g., 1, 2, 3, 4, 5, 6, 7, 8, 9, 10, 11, 12, 13, 14, 15, 16, 17, 18, 19, or 20 amino acids have been substituted, inserted, and/or deleted in SEQ ID NO: 76. In certain embodiments, the OX40 agonist antibody comprises a VH comprising one, two, or three HVRs selected from: (a) HVR-H1 comprising the amino acid sequence of SEQ ID NO: 22, (b) HVR-H2 comprising the amino acid sequence of SEQ ID NO: 23, and (c) HVR-H3 comprising the amino acid sequence of SEQ ID NO: 24.

In some embodiments, the OX40 agonist antibody comprises a VL having at least 80%, 81%, 82%, 83%, 84%, 85%, 86%, 87%, 88%, 89%, 90%, 91%, 92%, 93%, 94%, 95%, 96%, 97%, 98%, or 99% sequence identity to, or the sequence of, SEQ ID NO: 77. In some embodiments, the OX40 agonist antibody retains the ability to bind to human OX40. In some embodiments, a total of 1 to 20 amino acids have been substituted, inserted, and/or deleted in SEQ ID NO: 77, e.g., 1, 2, 3, 4, 5, 6, 7, 8, 9, 10, 11, 12, 13, 14, 15, 16, 17, 18, 19, or 20 amino acids have been substituted, inserted, and/or deleted in SEQ ID NO: 77. In some embodiments, the OX40 agonist antibody comprises a VL comprising one, two, or three HVRs selected from (a) HVR-L1 comprising the amino acid sequence of SEQ ID NO: 25; (b) HVR-L2 comprising the amino acid sequence of SEQ ID NO: 26; and (c) HVR-L3 comprising the amino acid sequence of SEQ ID NO: 27.

In some embodiments, the OX40 agonist antibody comprises a VH sequence of SEQ ID NO: 76. In some embodiments, the OX40 agonist antibody comprises a VL sequence of SEQ ID NO: 77. In certain embodiments, the OX40 agonist antibody comprises a VH sequence of SEQ ID NO: 76 and a VL sequence of SEQ ID NO: 77.

In some embodiments, the OX40 agonist antibody comprises a VH sequence of SEQ ID NO: 114. In some embodiments, the OX40 agonist antibody comprises a VL sequence of SEQ ID NO: 115. In certain embodiments, the OX40 agonist antibody comprises a VH sequence of SEQ ID NO: 114 and a VL sequence of SEQ ID NO: 115.

In some embodiments, the OX40 agonist antibody comprises a VH sequence of SEQ ID NO: 116. In some embodiments, the OX40 agonist antibody comprises a VL sequence of SEQ ID NO: 117. In certain embodiments, the OX40 agonist antibody comprises a VH sequence of SEQ ID NO: 116 and a VL sequence of SEQ ID NO: 117.

Table 1 provides sequence information for SEQ ID NOs: 22-117 mentioned above, as well as the sequence of human OX40 lacking the signal peptide (SEQ ID NO: 21).

TABLE 1
Sequences relating to selected OX40 agonist antibodies

| | | SEQ ID |
|---|---|---|
| Name | SEQUENCE | NO: |
| Human OX40 | LHCVGDTYPSNDRCCHECRPGNGMVSRCSRSQNTVCRPCGPGFY | 21 |
| (lacking the | NDVVSSKPCKPCTWCNLRSGSERKQLCTATQDTVCRCRAGTQPLD | |
| signal peptide) | SYKPGVDCAPCPPGHFSPGDNQACKPWTNCTLAGKHTLQPASNSS | |
| | DAICEDRDPPATQPQETQGPPARPITVQPTEAWPRTSQGPSTRPVE | |
| | VPGGRAVAAILGLGLVLGLLGPLAILLALYLLRRDQRLPPDAHKPPGG | |
| | GSFRTPIQEEQADAHSTLAKI | |
| HVR-H1- | DSYMS | 22 |
| 1A7.gr.1 | | |
| 1A7.gr.2 | | |
| 1A7.gr.3 | | |
| 1A7.gr.4 | | |
| 1A7.gr.5 | | |
| 1A7.gr.6 | | |
| 1A7.gr.7 | | |
| 1A7.gr.NADS | | |
| 1A7.gr.NADA | | |
| 1A7.gr.NGDA | | |
| 1A7.gr.SGDS | | |
| 1A7.gr.NGSS | | |
| 1A7.Ala.1 | | |
| 1A7.Ala.2 | | |
| 1A7.Ala.3 | | |
| 1A7.Ala.4 | | |
| 1A7.Ala.5 | | |
| 1A7.Ala.6 | | |
| 1A7.Ala.7 | | |
| 1A7.Ala.8 | | |
| 1A7.Ala.9 | | |
| 1A7.Ala.10 | | |
| 1A7.Ala.11 | | |
| 1A7.Ala.12 | | |
| 1A7.Ala.13 | | |
| 1A7.Ala.14 | | |
| 1A7.Ala.15 | | |
| 1A7.Ala.16 | | |
| HVR-H2- | DMYPDNGDSSYNQKFRE | 23 |
| 1A7.gr.1 | | |
| 1A7.gr.2 | | |
| 1A7.gr.3 | | |
| 1A7.gr.4 | | |
| 1A7.gr.5 | | |
| 1A7.gr.6 | | |
| 1A7.gr.7 | | |
| 1A7.gr.DA | | |
| 1A7.gr.ES | | |
| 1A7.Ala.1 | | |
| 1A7.Ala.2 | | |
| 1A7.Ala.3 | | |
| 1A7.Ala.4 | | |
| 1A7.Ala.5 | | |
| 1A7.Ala.6 | | |
| 1A7.Ala.7 | | |
| 1A7.Ala.8 | | |
| 1A7.Ala.9 | | |
| 1A7.Ala.10 | | |
| 1A7.Ala.11 | | |
| 1A7.Ala.12 | | |
| 1A7.Ala.13 | | |
| 1A7.Ala.14 | | |
| 1A7.Ala.15 | | |
| 1A7.Ala.16 | | |
| HVR-H3- | APRWYFSV | 24 |
| 1A7.gr.1 | | |
| 1A7.gr.2 | | |
| 1A7.gr.3 | | |
| 1A7.gr.4 | | |
| 1A7.gr.5 | | |
| 1A7.gr.6 | | |
| 1A7.gr.7 | | |
| 1A7.gr.DA | | |
| 1A7.gr.ES | | |
| 1A7.gr.NADS | | |
| 1A7.gr.NADA | | |
| 1A7.gr.NGDA | | |
| 1A7.gr.SGDS | | |
| 1A7.gr.NGSS | | |
| 1A7.gr.DANADA | | |
| 1A7.Ala.1 | | |
| 1A7.Ala.2 | | |
| 1A7.Ala.3 | | |
| 1A7.Ala.4 | | |
| 1A7.Ala.5 | | |
| 1A7.Ala.6 | | |
| 1A7.Ala.7 | | |
| 1A7-Ala.15 | | |
| 1A7.Ala.16 | | |
| HVR-L1- | RASQDISNYLN | 25 |
| 1A7.gr.1 | | |
| 1A7.gr.2 | | |
| 1A7.gr.3 | | |
| 1A7.gr.4 | | |
| 1A7.gr.5 | | |
| 1A7.gr.6 | | |
| 1A7.gr.7 | | |
| 1A7.grDA | | |
| 1A7.grES | | |
| 1A7.gr.NADS | | |
| 1A7.gr.NADA | | |
| 1A7.gr.NGDA | | |
| 1A7.gr.SGDS | | |
| 1A7.gr.NGSS | | |
| 1A7.gr.DANADA | | |
| 1A7.Ala.1 | | |
| 1A7.Ala.2 | | |
| 1A7.Ala.3 | | |
| 1A7.Ala.4 | | |
| 1A7.Ala.5 | | |
| 1A7.Ala.6 | | |
| 1A7.Ala.7 | | |
| 1A7.Ala.8 | | |
| 1A7.Ala.9 | | |
| 1A7.Ala.10 | | |
| 1A7.Ala.11 | | |
| 1A7.Ala.12 | | |
| 1A7.Ala.13 | | |
| 1A7.Ala.14 | | |
| 1A7.Ala.15 | | |
| 1A7.Ala.16 | | |
| HVR-L2- | YTSRLRS | 26 |
| 1A7.gr.1 | | |
| 1A7.gr.2 | | |
| 1A7.gr.3 | | |
| 1A7.gr.4 | | |
| 1A7.gr.5 | | |
| 1A7.gr.6 | | |
| 1A7.gr.7 | | |
| 1A7.grDA | | |
| 1A7.grES | | |
| 1A7.gr.NADS | | |
| 1A7.gr.NADA | | |
| 1A7.gr.NGDA | | |
| 1A7.gr.SGDS | | |
| 1A7.gr.NGSS | | |
| 1A7.gr.DANADA | | |
| 1A7.Ala.1 | | |
| 1A7.Ala.2 | | |
| 1A7.Ala.3 | | |
| 1A7.Ala.4 | | |
| 1A7.Ala.5 | | |
| 1A7.Ala.6 | | |
| 1A7.Ala.7 | | |
| 1A7.Ala.8 | | |
| 1A7.Ala.9 | | |
| 1A7.Ala.10 | | |
| 1A7.Ala.11 | | |
| 1A7.Ala.12 | | |
| 1A7.Ala.13 | | |
| 1A7.Ala.14 | | |
| 1A7.Ala.15 | | |
| 1A7.Ala.16 | | |
| HVR-L3- | QQGHTLPPT | 27 |
| 1A7.gr.1 | | |
| 1A7.gr.2 | | |
| 1A7.gr.3 | | |
| 1A7.gr.4 | | |
| 1A7.gr.5 | | |
| 1A7.gr.6 | | |
| 1A7.gr.7 | | |
| 1A7.gr.DA | | |
| 1A7.gr.ES | | |
| 1A7.gr.NADS | | |
| 1A7.gr.NADA | | |
| 1A7.gr.NGDA | | |
| 1A7.gr.SGDS | | |
| 1A7.gr.NGSS | | |
| 1A7.gr.DANADA | | |
| 1A7.Ala.8 | | |
| 1A7.Ala.9 | | |
| 1A7.Ala.10 | | |
| 1A7.Ala.11 | | |
| 1A7.Ala.12 | | |
| 1A7.Ala.13 | | |
| 1A7.Ala.14 | | |
| 1A7.Ala.15 | | |
| 1A7.Ala.16 | | |
| HVR-H1- | DAYMS | 28 |
| 1A7.gr.DA | | |
| HVR-H1- | ESYMS | 29 |
| 1A7.gr.ES | | |
| 1A7.gr.DANADA | | |
| HVR-H2- | DMYPDNADSSYNQKFRE | 30 |
| 1A7.gr.NADS | | |
| HVR-H2- | DMYPDNADASYNQKFRE | 31 |
| 1A7.gr.NADA | | |
| 1A7.gr.DANADA | | |
| HVR-H2- | DMYPDNGDASYNQKFRE | 32 |
| 1A7.gr.NGDA | | |
| HVR-H2- | DMYPDSGDSSYNQKFRE | 33 |
| 1A7.gr.SGDS | | |
| HVR-H2- | DMYPDNGSSSYNQKFRE | 34 |
| 1A7.gr.NGSS | | |
| HVR-H3- | APRWYFSA | 35 |
| 1A7.Ala.8 | | |
| HVR-H3- | APRWYASV | 36 |
| 1A7.Ala.9 | | |
| HVR-H3- | APRWAFSV | 37 |
| 1A7.Ala.10 | | |
| HVR-H3- | APAWYFSV | 38 |
| 1A7.Ala.11 | | |
| HVR-H3- | APRWYFAV | 39 |
| 1A7.Ala.12 | | |
| HVR-H3- | APRAYFSV | 40 |
| 1A7.Ala.13 | | |
| HVR-H3- | AARWYFSV | 41 |
| 1A7.Ala.14 | | |
| HVR-L3- | QQGHTLPAT | 42 |
| 1A7.Ala.1 | | |
| HVR-L3- | QQGHTAPPT | 43 |
| 1A7.Ala.2 | | |
| HVR-L3- | QQGATLPPT | 44 |
| 1A7.Ala.3 | | |
| HVR-L3- | QQGHALPPT | 45 |
| 1A7.Ala.4 | | |
| HVR-L3- | QQAHTLPPT | 46 |
| 1A7.Ala.5 | | |
| HVR-L3- | QQGHTLAPT | 47 |
| 1A7.Ala.6 | | |
| HVR-L3- | QAGHTLPPT | 48 |
| 1A7.Ala.7 | | |
| HVR-H1- | NYLIE | 49 |
| 3C8.gr.1 | | |
| 3C8.gr.2 | | |
| 3C8.gr.3 | | |
| 3C8.gr.4 | | |
| 3C8.gr.5 | | |
| 3C8.gr.5.SG | | |
| 3C8.gr.5.EG | | |
| 3C8.gr.5.QG | | |
| 3C9.gr.5.DQ | | |
| 3C8.gr.5.DA | | |
| 3C8.gr.6 | | |
| 3C8.gr.7 | | |
| 3C8.gr.8 | | |
| 3C8.gr.9 | | |
| 3C8.gr.10 | | |
| 3C8.gr.11 | | |
| 3C8.A.1 | | |
| 3C8.A.2 | | |
| 3C8.A.3 | | |
| 3C8.A.4 | | |
| 3C8.A.5 | | |
| 3C8.A.6 | | |
| 3C8.A.7 | | |
| 3C8.A.8 | | |
| 3C8.A.9 | | |
| 3C8.A.10 | | |
| HVR-H2- | VINPGSGDTYYSEKFKG | 50 |
| 3C8.gr.1 | | |
| 3C8.gr.2 | | |
| 3C8.gr.3 | | |
| 3C8.gr.4 | | |
| 3C8.gr.5 | | |
| 3C8.gr.5.SG | | |
| 3C8.gr.5.EG | | |
| 3C8.gr.5.QG | | |
| 3C8.gr.6 | | |
| 3C8.gr.7 | | |
| 3C8.gr.8 | | |
| 3C8.gr.9 | | |
| 3C8.gr.10 | | |
| 3C8.gr.11 | | |
| 3C8.A.1 | | |
| 3C8.A.2 | | |
| 3C8.A.3 | | |
| 3C8.A.4 | | |
| 3C8.A.5 | | |
| 3C8.A.6 | | |
| 3C8.A.7 | | |
| 3C8.A.8 | | |
| 3C8.A.9 | | |
| 3C8.A.10 | | |
| HVR-H2- | VINPGSGDAYYSEKFKG | 51 |
| 3C8.gr.5.DA | | |
| HVR-H2- | VINPGSGDQYYSEKFKG | 52 |
| 3C8.gr.5.DQ | | |
| HVR-H3- | DRLDY | 53 |
| 3C8.gr.1 | | |
| 3C8.gr.2 | | |
| 3C8.gr.3 | | |
| 3C8.gr.4 | | |
| 3C8.gr.5 | | |
| 3C8.gr.5.SG | | |
| 3C8.gr.5.EG | | |
| 3C8.gr.5.QG | | |
| 3C8.gr.5.DA | | |
| 3C8.gr.5.DQ | | |
| 3C8.gr.6 | | |
| 3C8.gr.7 | | |
| 3C8.gr.8 | | |
| 3C8.gr.9 | | |
| 3C8.gr.10 | | |
| 3C8.gr.11 | | |
| 3C8.A.1 | | |
| 3C8.A.2 | | |
| 3C8.A.3 | | |
| 3C8.A.4 | | |
| 3C8.A.5 | | |
| 3C8.A.6 | | |
| 3C8.A.7 | | |
| HVR-H3- | ARLDY | 54 |
| 3C8.A.8 | | |
| HVR-H3- | DALDY | 55 |
| 3C8.A.9 | | |
| HVR-H3- | DRADY | 56 |
| 3C8.A.10 | | |
| HVR-L1- | HASQDISSYIV | 57 |
| 3C8.gr.1 | | |
| 3C8.gr.2 | | |
| 3C8.gr.3 | | |
| 3C8.gr.4 | | |
| 3C8.gr.5 | | |
| 3C8.gr.5.SG | | |
| 3C8.gr.5.EG | | |
| 3C8.gr.5.QG | | |
| 3C8.gr.5.DA | | |
| 3C8.gr.5.DQ | | |
| 3C8.gr.6 | | |
| 3C8.gr.7 | | |
| 3C8.gr.8 | | |
| 3C8.gr.9 | | |
| 3C8.gr.10 | | |
| 3C8.gr.11 | | |
| 3C8.A.1 | | |
| 3C8.A.2 | | |
| 3C8.A.3 | | |
| 3C8.A.4 | | |
| 3C8.A.5 | | |
| 3C8.A.6 | | |
| 3C8.A.7 | | |
| 3C8.A.8 | | |
| 3C8.A.9 | | |
| 3C8.A.10 | | |
| HVR-L2- | HGTNLED | 58 |
| 3C8.gr.1 | | |
| 3C8.gr.2 | | |
| 3C8.gr.3 | | |
| 3C8.gr.4 | | |
| 3C8.gr.5 | | |
| 3C8.gr.5.DA | | |
| 3C8.gr.5.DQ | | |
| 3C8.gr.6 | | |
| 3C8.gr.7 | | |
| 3C8.gr.8 | | |
| 3C8.gr.9 | | |
| 3C8.gr.10 | | |
| 3C8.gr.11 | | |
| 3C8.A.1 | | |
| 3C8.A.2 | | |
| 3C8.A.3 | | |
| 3C8.A.4 | | |
| 3C8.A.5 | | |
| 3C8.A.6 | | |
| 3C8.A.7 | | |
| 3C8.A.8 | | |
| 3C8.A.9 | | |
| 3C8.A.10 | | |
| HVR-L2- | HGTNLES | 59 |
| 3C8.gr5.SG | | |
| HVR-L2- | HGTNLEE | 60 |
| 3C8.gr.5.EG | | |
| HVR-L2- | HGTNLEQ | 61 |
| 3C8.gr.5.QG | | |
| HVR-L3 | VHYAQFPYT | 62 |
| 3C8.gr.1 | | |
| 3C8.gr.2 | | |
| 3C8.gr.3 | | |
| 3C8.gr.4 | | |
| 3C8.gr.5 | | |
| 3C8.gr.5.SG | | |
| 3C8.gr.5.EG | | |
| 3C8.gr.5.QG | | |
| 3C8.gr.5.DA | | |
| 3C8.gr.5.DQ | | |
| 3C8.gr.6 | | |
| 3C8.gr.7 | | |
| 3C8.gr.8 | | |
| 3C8.gr.9 | | |
| 3C8.gr.10 | | |
| 3C8.gr.11 | | |
| 3C8.A.8 | | |
| 3C8.A.9 | | |
| 3C8.A.10 | | |
| HVR-L3- | AHYAQFPYT | 63 |
| 3C8.A.1 | | |
| HVR-L3- | VAYAQFPYT | 64 |
| 3C8.A.2 | | |
| HVR-L3- | VHAAQFPYT | 65 |
| 3C8.A.3 | | |
| HVR-L3- | VHYAAFPYT | 66 |
| 3C8.A.4 | | |
| HVR-L3- | VHYAQAPYT | 67 |
| 3C8.A.5 | | |
| HVR-L3- | VHYAQFAYT | 68 |
| 3C8.A.6 | | |
| HVR-L3- | VHYAQFPAT | 69 |
| 3C8.A.7 | | |
| HVR-H1- | DYGVL | 70 |
| 1D2.gr.1 | | |
| 1D2.gr.2 | | |
| 1D2.gr.3 | | |
| HVR-H2- | MIWSGGTTDYNAAFIS | 71 |
| 1D2.gr.1 | | |
| 1D2.gr.2 | | |
| 1D2.gr.3 | | |
| HVR-H3- | EEMDY | 72 |
| 1D2.gr.1 | | |
| 1D2.gr.2 | | |
| 1D2.gr.3 | | |
| HVR-L1- | RASQDISNFLN | 73 |
| 1D2.gr.1 | | |
| 1D2.gr.2 | | |
| 1D2.gr.3 | | |
| HVR-L2- | YTSRLHS | 74 |
| 1D2.gr.1 | | |
| 1D2.gr.2 | | |
| 1D2.gr.3 | | |
| HVR-L3- | QQGNTLPWT | 75 |
| 1D2.gr.1 | | |
| 1D2.gr.2 | | |
| 1D2.gr.3 | | |
| 1A7.gr.1 | EVQLVQSGAEVKKPGASVKVSCKASGYTFTDSYMSWVRQAPGQGL | 76 |
| V_{H} | EWIGDMYPDNGDSSYNQKFRERVTITRDTSTSTAYLELSSLRSEDTA | |
| | VYYCVLAPRWYFSVWGQGTLVTVSS | |
| 1A7.gr.1 | DIQMTQSPSSLSASVGDRVTITCRASQDISNYLNWYQQKPGKAPKLL | 77 |
| V_{L} | IYYTSRLRSGVPSRFSGSGSGTDFTLTISSLQPEDFATYYCQQGHTL | |
| | PPTFGQGTKVEIK | |
| 1A7.gr.2 | EVQLVQSGAEVKKPGASVKVSCKASGYTFTDSYMSWVRQAPGQGL | 78 |
| V_{H} | EWIGDMYPDNGDSSYNQKFRERVTITVDTSTSTAYLELSSLRSEDTA | |
| | VYYCVLAPRWYFSVWGQGTLVTVSS | |
| 1A7.gr.2 | DIQMTQSPSSLSASVGDRVTITCRASQDISNYLNWYQQKPGKAPKLL | 79 |
| V_{L} | IYYTSRLRSGVPSRFSGSGSGTDFTLTISSLQPEDFATYYCQQGHTL | |
| | PPTFGQGTKVEIK | |
| 1A7.gr.3 | EVQLVQSGAEVKKPGASVKVSCKASGYTFTDSYMSWVRQAPGQGL | 80 |
| V_{H} | EWIGDMYPDNGDSSYNQKFRERVTLTVDTSTSTAYLELSSLRSEDT | |
| | AVYYCVLAPRWYFSVWGQGTLVTVSS | |
| 1A7.gr.3 | DIQMTQSPSSLSASVGDRVTITCRASQDISNYLNWYQQKPGKAPKLL | 81 |
| V_{L} | IYYTSRLRSGVPSRFSGSGSGTDFTLTISSLQPEDFATYYCQQGHTL | |
| | PPTFGQGTKVEIK | |
| 1A7.gr.4 | EVQLVQSGAEVKKPGASVKVSCKASGYTFTDSYMSWVRQAPGQGL | 82 |
| V_{H} | EWIGDMYPDNGDSSYNQKFRERVTITVDTSTSTAYLELSSLRSEDTA | |
| | VYYCVLAPRWYFSVWGQGTLVTVSS | |
| 1A7.gr.4 | DIQMTQSPSSLSASVGDRVTITCRASQDISNYLNWYQQKPGKTVKLL | 83 |
| V_{L} | IYYTSRLRSGVPSRFSGSGSGTDFTLTISSLQPEDFATYYCQQGHTL | |
| | PPTFGQGTKVEIK | |
| 1A7.gr.5 | EVQLVQSGAEVKKPGASVKVSCKASGYTFTDSYMSWVRQAPGQGL | 84 |
| V_{H} | EWIGDMYPDNGDSSYNQKFRERVTITVDTSTSTAYLELSSLRSEDTA | |
| | VYYCVLAPRWYFSVWGQGTLVTVSS | |
| 1A7.gr.5 | DIQMTQSPSSLSASVGDRVTITCRASQDISNYLNWYQQKPGKTVKLL | 85 |
| V_{L} | IYYTSRLRSGVPSRFSGSGSGTDFTLTISSLQPEDFATYYCQQGHTL | |
| | PPTFGQGTKVEIK | |
| 1A7.gr.6 | EVQLVQSGAEVKKPGASVKVSCKASGYTFTDSYMSWVRQAPGQGL | 86 |
| V_{H} | EWIGDMYPDNGDSSYNQKFRERVTITVDTSTSTAYLELSSLRSEDTA | |
| | VYYCVLAPRWYFSVWGQGTLVTVSS | |
| 1A7.gr.6 | DIQMTQSPSSLSASVGDRVTITCRASQDISNYLNWYQQKPGKTVKLL | 87 |
| V_{L} | IYYTSRLRSGVPSRFSGSGSGKDYTLTISSLQPEDFATYFCQQGHTL | |
| | PPTFGQGTKVEIK | |
| 1A7.gr.7 | EVQLVQSGAEVKKPGASVKVSCKASGYTFTDSYMSWVRQAPGQGL | 88 |
| V_{H} | EWIGDMYPDNGDSSYNQKFRERVTITVDTSTSTAYLELSSLRSEDTA | |
| | VYYCVLAPRWYFSVWGQGTLVTVSS | |
| 1A7.gr.7 | DIQMTQSPSSLSASVGDRVTITCRASQDISNYLNWYQQKPGKTVKLL | 89 |
| V_{L} | IYYTSRLRSGVPSRFSGSGSGKDYTLTISSLQPEDFATYFCQQGHTL | |
| | PPTFGQGTKVEIK | |
| 1A7.gr.DA | EVQLVQSGAEVKKPGASVKVSCKASGYTFTDAYMSWVRQAPGQGL | 90 |
| V_{H} | EWIGDMYPDNGDSSYNQKFRERVTITRDTSTSTAYLELSSLRSEDTA | |
| | VYYCVLAPRWYFSVWGQGTLVTVSS | |
| 1A7.gr.DA | DIQMTQSPSSLSASVGDRVTITCRASQDISNYLNWYQQKPGKAPKLL | 91 |
| V_{L} | IYYTSRLRSGVPSRFSGSGSGTDFTLTISSLQPEDFATYYCQQGHTL | |
| | PPTFGQGTKVEIK | |
| 1A7.gr.ES | EVQLVQSGAEVKKPGASVKVSCKASGYTFTESYMSWVRQAPGQGL | 92 |
| V_{H} | EWIGDMYPDNGDSSYNQKFRERVTITRDTSTSTAYLELSSLRSEDTA | |
| | VYYCVLAPRWYFSVWGQGTLVTVSS | |
| 1A7.gr.ES | DIQMTQSPSSLSASVGDRVTITCRASQDISNYLNWYQQKPGKAPKLL | 93 |
| V_{L} | IYYTSRLRSGVPSRFSGSGSGTDFTLTISSLQPEDFATYYCQQGHTL | |
| | PPTFGQGTKVEIK | |
| 1A7.gr.NADS | EVQLVQSGAEVKKPGASVKVSCKASGYTFTDSYMSWVRQAPGQGL | 94 |
| V_{H} | EWIGDMYPDNADSSYNQKFRERVTITRDTSTSTAYLELSSLRSEDTA | |
| | VYYCVLAPRWYFSVWGQGTLVTVSS | |
| 1A7.gr.NADS | DIQMTQSPSSLSASVGDRVTITCRASQDISNYLNWYQQKPGKAPKLL | 95 |
| V_{L} | IYYTSRLRSGVPSRFSGSGSGTDFTLTISSLQPEDFATYYCQQGHTL | |
| | PPTFGQGTKVEIK | |
| 1A7.gr.NADA | EVQLVQSGAEVKKPGASVKVSCKASGYTFTDSYMSWVRQAPGQGL | 96 |
| V_{H} | EWIGDMYPDNADASYNQKFRERVTITRDTSTSTAYLELSSLRSEDTA | |
| | VYYCVLAPRWYFSVWGQGTLVTVSS | |
| 1A7.gr.NADA | DIQMTQSPSSLSASVGDRVTITCRASQDISNYLNWYQQKPGKAPKLL | 97 |
| V_{L} | IYYTSRLRSGVPSRFSGSGSGTDFTLTISSLQPEDFATYYCQQGHTL | |
| | PPTFGQGTKVEIK | |
| 1A7.gr.NGDA | EVQLVQSGAEVKKPGASVKVSCKASGYTFTDSYMSWVRQAPGQGL | 98 |
| V_{H} | EWIGDMYPDNGDASYNQKFRERVTITRDTSTSTAYLELSSLRSEDTA | |
| | VYYCVLAPRWYFSVWGQGTLVTVSS | |
| 1A7.gr.NGDA | DIQMTQSPSSLSASVGDRVTITCRASQDISNYLNWYQQKPGKAPKLL | 99 |
| V_{L} | IYYTSRLRSGVPSRFSGSGSGTDFTLTISSLQPEDFATYYCQQGHTL | |
| | PPTFGQGTKVEIK | |
| 1A7.gr.SGDS | EVQLVQSGAEVKKPGASVKVSCKASGYTFTDSYMSWVRQAPGQGL | 100 |
| V_{H} | EWIGDMYPDSGDSSYNQKFRERVTITRDTSTSTAYLELSSLRSEDTA | |
| | VYYCVLAPRWYFSVWGQGTLVTVSS | |
| 1A7.gr.SGDS | DIQMTQSPSSLSASVGDRVTITCRASQDISNYLNWYQQKPGKAPKLL | 101 |
| V_{L} | IYYTSRLRSGVPSRFSGSGSGTDFTLTISSLQPEDFATYYCQQGHTL | |
| | PPTFGQGTKVEIK | |
| 1A7.gr.NGSS | EVQLVQSGAEVKKPGASVKVSCKASGYTFTDSYMSWVRQAPGQGL | 102 |
| V_{H} | EWIGDMYPDNGSSSYNQKFRERVTITRDTSTSTAYLELSSLRSEDTA | |
| | VYYCVLAPRWYFSVWGQGTLVTVSS | |
| 1A7.gr.NGSS | DIQMTQSPSSLSASVGDRVTITCRASQDISNYLNWYQQKPGKAPKLL | 103 |
| V_{L} | IYYTSRLRSGVPSRFSGSGSGTDFTLTISSLQPEDFATYYCQQGHTL | |
| | PPTFGQGTKVEIK | |
| 1A7.gr.DANADA | EVQLVQSGAEVKKPGASVKVSCKASGYTFTDAYMSWVRQAPGQGL | 104 |
| V_{H} | EWIGDMYPDNADASYNQKFRERVTITRDTSTSTAYLELSSLRSEDTA | |
| | VYYCVLAPRWYFSVWGQGTLVTVSS | |
| 1A7.gr.DANADA | DIQMTQSPSSLSASVGDRVTITCRASQDISNYLNWYQQKPGKAPKLL | 105 |
| V_{L} | IYYTSRLRSGVPSRFSGSGSGTDFTLTISSLQPEDFATYYCQQGHTL | |
| | PPTFGQGTKVEIK | |
| 1A7.Ala.1 | EVQLVQSGAEVKKPGASVKVSCKASGYTFTDSYMSWVRQAPGQGL | 106 |
| V_{H} | EWIGDMYPDNGDSSYNQKFRERVTITRDTSTSTAYLELSSLRSEDTA | |
| | VYYCVLAPRWYFSVWGQGTLVTVSS | |
| 1A7.Ala.1 | DIQMTQSPSSLSASVGDRVTITCRASQDISNYLNWYQQKPGKAPKLL | 107 |
| V_{L} | IYYTSRLRSGVPSRFSGSGSGTDFTLTISSLQPEDFATYYCQQGHTL | |
| | PATFGQGTKVEIK | |
| 1A7.Ala.2 | EVQLVQSGAEVKKPGASVKVSCKASGYTFTDSYMSWVRQAPGQGL | 108 |
| V_{H} | EWIGDMYPDNGDSSYNQKFRERVTITRDTSTSTAYLELSSLRSEDTA | |
| | VYYCVLAPRWYFSVWGQGTLVTVSS | |
| 1A7.Ala.2 | DIQMTQSPSSLSASVGDRVTITCRASQDISNYLNWYQQKPGKAPKLL | 109 |
| V_{L} | IYYTSRLRSGVPSRFSGSGSGTDFTLTISSLQPEDFATYYCQQGHTA | |
| | PPTFGQGTKVEIK | |
| 1A7.Ala.3 | EVQLVQSGAEVKKPGASVKVSCKASGYTFTDSYMSWVRQAPGQGL | 110 |
| V_{H} | EWIGDMYPDNGDSSYNQKFRERVTITRDTSTSTAYLELSSLRSEDTA | |
| | VYYCVLAPRWYFSVWGQGTLVTVSS | |
| 1A7.Ala.3 | DIQMTQSPSSLSASVGDRVTITCRASQDISNYLNWYQQKPGKAPKLL | 111 |
| V_{L} | IYYTSRLRSGVPSRFSGSGSGTDFTLTISSLQPEDFATYYCQQGATL | |
| | PPTFGQGTKVEIK | |
| 1A7.Ala.4 | EVQLVQSGAEVKKPGASVKVSCKASGYTFTDSYMSWVRQAPGQGL | 112 |
| V_{H} | EWIGDMYPDNGDSSYNQKFRERVTITRDTSTSTAYLELSSLRSEDTA | |
| | VYYCVLAPRWYFSVWGQGTLVTVSS | |
| 1A7.Ala.4 | DIQMTQSPSSLSASVGDRVTITCRASQDISNYLNWYQQKPGKAPKLL | 113 |
| V_{L} | IYYTSRLRSGVPSRFSGSGSGTDFTLTISSLQPEDFATYYCQQGHAL | |
| | PPTFGQGTKVEIK | |
| 1A7.Ala.5 | EVQLVQSGAEVKKPGASVKVSCKASGYTFTDSYMSWVRQAPGQGL | 114 |
| V_{H} | EWIGDMYPDNGDSSYNQKFRERVTITRDTSTSTAYLELSSLRSEDTA | |
| | VYYCVLAPRWYFSVWGQGTLVTVSS | |
| 1A7.Ala.5 | DIQMTQSPSSLSASVGDRVTITCRASQDISNYLNWYQQKPGKAPKLL | 115 |
| V_{L} | IYYTSRLRSGVPSRFSGSGSGTDFTLTISSLQPEDFATYYCQQAHTL | |
| | PPTFGQGTKVEIK | |
| 1A7.Ala.6 | EVQLVQSGAEVKKPGASVKVSCKASGYTFTDSYMSWVRQAPGQGL | 116 |
| V_{H} | EWIGDMYPDNGDSSYNQKFRERVTITRDTSTSTAYLELSSLRSEDTA | |
| | VYYCVLAPRWYFSVWGQGTLVTVSS | |
| 1A7.Ala.6 | DIQMTQSPSSLSASVGDRVTITCRASQDISNYLNWYQQKPGKAPKLL | 117 |
| V_{L} | IYYTSRLRSGVPSRFSGSGSGTDFTLTISSLQPEDFATYYCQQGHTL | |
| | APTFGQGTKVEIK | |
| 1A7.Ala.7 | EVQLVQSGAEVKKPGASVKVSCKASGYTFTDSYMSWVRQAPGQGL | 118 |
| V_{H} | EWIGDMYPDNGDSSYNQKFRERVTITRDTSTSTAYLELSSLRSEDTA | |
| | VYYCVLAPRWYFSVWGQGTLVTVSS | |
| 1A7.Ala.7 | DIQMTQSPSSLSASVGDRVTITCRASQDISNYLNWYQQKPGKAPKLL | 119 |
| V_{L} | IYYTSRLRSGVPSRFSGSGSGTDFTLTISSLQPEDFATYYCQAGHTL | |
| | PPTFGQGTKVEIK | |
| 1A7.Ala.8 | EVQLVQSGAEVKKPGASVKVSCKASGYTFTDSYMSWVRQAPGQGL | 120 |
| V_{H} | EWIGDMYPDNGDSSYNQKFRERVTITRDTSTSTAYLELSSLRSEDTA | |
| | VYYCVLAPRWYFSAWGQGTLVTVSS | |
| 1A7.Ala.8 | DIQMTQSPSSLSASVGDRVTITCRASQDISNYLNWYQQKPGKAPKLL | 121 |
| V_{L} | IYYTSRLRSGVPSRFSGSGSGTDFTLTISSLQPEDFATYYCQQGHTL | |
| | PPTFGQGTKVEIK | |
| 1A7.Ala.9 | EVQLVQSGAEVKKPGASVKVSCKASGYTFTDSYMSWVRQAPGQGL | 122 |
| V_{H} | EWIGDMYPDNGDSSYNQKFRERVTITRDTSTSTAYLELSSLRSEDTA | |
| | VYYCVLAPRWYASVWGQGTLVTVSS | |
| 1A7.Ala.9 | DIQMTQSPSSLSASVGDRVTITCRASQDISNYLNWYQQKPGKAPKLL | 123 |
| V_{L} | IYYTSRLRSGVPSRFSGSGSGTDFTLTISSLQPEDFATYYCQQGHTL | |
| | PPTFGQGTKVEIK | |
| 1A7.Ala.10 | EVQLVQSGAEVKKPGASVKVSCKASGYTFTDSYMSWVRQAPGQGL | 124 |
| V_{H} | EWIGDMYPDNGDSSYNQKFRERVTITRDTSTSTAYLELSSLRSEDTA | |
| | VYYCVLAPRWAFSVWGQGTLVTVSS | |
| 1A7.Ala.10 | DIQMTQSPSSLSASVGDRVTITCRASQDISNYLNWYQQKPGKAPKLL | 125 |
| V_{L} | IYYTSRLRSGVPSRFSGSGSGTDFTLTISSLQPEDFATYYCQQGHTL | |
| | PPTFGQGTKVEIK | |
| 1A7.Ala.11 | EVQLVQSGAEVKKPGASVKVSCKASGYTFTDSYMSWVRQAPGQGL | 126 |
| V_{H} | EWIGDMYPDNGDSSYNQKFRERVTITRDTSTSTAYLELSSLRSEDTA | |
| | VYYCVLAPAWYFSVWGQGTLVTVSS | |
| 1A7.Ala.11 | DIQMTQSPSSLSASVGDRVTITCRASQDISNYLNWYQQKPGKAPKLL | 127 |
| V_{L} | IYYTSRLRSGVPSRFSGSGSGTDFTLTISSLQPEDFATYYCQQGHTL | |
| | PPTFGQGTKVEIK | |
| 1A7.Ala.12 | EVQLVQSGAEVKKPGASVKVSCKASGYTFTDSYMSWVRQAPGQGL | 128 |
| V_{H} | EWIGDMYPDNGDSSYNQKFRERVTITRDTSTSTAYLELSSLRSEDTA | |
| | VYYCVLAPRWYFAVWGQGTLVTVSS | |
| 1A7.Ala.12 | DIQMTQSPSSLSASVGDRVTITCRASQDISNYLNWYQQKPGKAPKLL | 129 |
| V_{L} | IYYTSRLRSGVPSRFSGSGSGTDFTLTISSLQPEDFATYYCQQGHTL | |
| | PPTFGQGTKVEIK | |
| 1A7.Ala.13 | EVQLVQSGAEVKKPGASVKVSCKASGYTFTDSYMSWVRQAPGQGL | 130 |
| V_{H} | EWIGDMYPDNGDSSYNQKFRERVTITRDTSTSTAYLELSSLRSEDTA | |
| | VYYCVLAPRAYFSVWGQGTLVTVSS | |
| 1A7.Ala.13 | DIQMTQSPSSLSASVGDRVTITCRASQDISNYLNWYQQKPGKAPKLL | 131 |
| V_{L} | IYYTSRLRSGVPSRFSGSGSGTDFTLTISSLQPEDFATYYCQQGHTL | |
| | PPTFGQGTKVEIK | |
| 1A7.Ala.14 | EVQLVQSGAEVKKPGASVKVSCKASGYTFTDSYMSWVRQAPGQGL | 132 |
| V_{H} | EWIGDMYPDNGDSSYNQKFRERVTITRDTSTSTAYLELSSLRSEDTA | |
| | VYYCVLAARWYFSVWGQGTLVTVSS | |
| 1A7.Ala.14 | DIQMTQSPSSLSASVGDRVTITCRASQDISNYLNWYQQKPGKAPKLL | 133 |
| V_{L} | IYYTSRLRSGVPSRFSGSGSGTDFTLTISSLQPEDFATYYCQQGHTL | |
| | PPTFGQGTKVEIK | |
| 1A7.Ala.15 | EVQLVQSGAEVKKPGASVKVSCKASGYTFTDSYMSWVRQAPGQGL | 134 |
| V_{H} | EWIGDMYPDNGDSSYNQKFRERVTITRDTSTSTAYLELSSLRSEDTA | |
| | VYYCALAPRWYFSVWGQGTLVTVSS | |
| 1A7.Ala.15 | DIQMTQSPSSLSASVGDRVTITCRASQDISNYLNWYQQKPGKAPKLL | 135 |
| V_{L} | IYYTSRLRSGVPSRFSGSGSGTDFTLTISSLQPEDFATYYCQQGHTL | |
| | PPTFGQGTKVEIK | |
| 1A7.Ala.16 | EVQLVQSGAEVKKPGASVKVSCKASGYTFTDSYMSWVRQAPGQGL | 136 |
| V_{H} | EWIGDMYPDNGDSSYNQKFRERVTITRDTSTSTAYLELSSLRSEDTA | |
| | VYYCVAAPRWYFSVWGQGTLVTVSS | |
| 1A7.Ala.16 | DIQMTQSPSSLSASVGDRVTITCRASQDISNYLNWYQQKPGKAPKLL | 137 |
| V_{L} | IYYTSRLRSGVPSRFSGSGSGTDFTLTISSLQPEDFATYYCQQGHTL | |
| | PPTFGQGTKVEIK | |
| 3C8.gr.1 | EVQLVQSGAEVKKPGASVKVSCKASGYAFTNYLIEWVRQAPGQGL | 138 |
| V_{H} | EWIGVINPGSGDTYYSEKFKGRVTITRDTSTSTAYLELSSLRSEDTAV | |
| | YYCARDRLDYWGQGTLVTVSS | |
| 3C8.gr.1 | DIQMTQSPSSLSASVGDRVTITCHASQDISSYIVWYQQKPGKAPKLLI | 139 |
| V_{L} | YHGTNLEDGVPSRFSGSGSGTDFTLTISSLQPEDFATYYCVHYAQF | |
| | PYTFGQGTKVEIK | |
| 3C8.gr.2 | EVQLVQSGAEVKKPGASVKVSCKASGYAFTNYLIEWVRQAPGQGL | 140 |
| V_{H} | EWIGVINPGSGDTYYSEKFKGRVTITADTSTSTAYLELSSLRSEDTAV | |
| | YYCARDRLDYWGQGTLVTVSS | |
| 3C8.gr.2 | DIQMTQSPSSLSASVGDRVTITCHASQDISSYIVWYQQKPGKAPKLLI | 141 |
| V_{L} | YHGTNLEDGVPSRFSGSGSGTDFTLTISSLQPEDFATYYCVHYAQF | |
| | PYTFGQGTKVEIK | |
| 3C8.gr.3 | EVQLVQSGAEVKKPGASVKVSCKASGYAFTNYLIEWVRQAPGQGL | 142 |
| V_{H} | EWIGVINPGSGDTYYSEKFKGRVTLTADTSTSTAYLELSSLRSEDTA | |
| | VYYCARDRLDYWGQGTLVTVSS | |
| 3C8.gr.3 | DIQMTQSPSSLSASVGDRVTITCHASQDISSYIVWYQQKPGKAPKLLI | 143 |
| V_{L} | YHGTNLEDGVPSRFSGSGSGTDFTLTISSLQPEDFATYYCVHYAQF | |
| | PYTFGQGTKVEIK | |
| 3C8.gr.4 | EVQLVQSGAEVKKPGASVKVSCKASGYAFTNYLIEWVRQAPGQGL | 144 |
| V_{H} | EWIGVINPGSGDTYYSEKFKGRVTITADTSTSTAYLELSSLRSEDTAV | |
| | YYCARDRLDYWGQGTLVTVSS | |
| 3C8.gr.4 | DIQMTQSPSSLSASVGDRVTITCHASQDISSYIVWYQQKPGKSFKGL | 145 |
| V_{L} | IYHGTNLEDGVPSRFSGSGSGTDFTLTISSLQPEDFATYYCVHYAQF | |
| | PYTFGQGTKVEIK | |
| 3C8.gr.5 | EVQLVQSGAEVKKPGASVKVSCKASGYAFTNYLIEWVRQAPGQGL | 146 |
| V_{H} | EWIGVINPGSGDTYYSEKFKGRVTLTADTSTSTAYLELSSLRSEDTA | |
| | VYYCARDRLDYWGQGTLVTVSS | |
| 3C8.gr.5 | DIQMTQSPSSLSASVGDRVTITCHASQDISSYIVWYQQKPGKSFKGL | 147 |
| V_{L} | IYHGTNLEDGVPSRFSGSGSGTDFTLTISSLQPEDFATYYCVHYAQF | |
| | PYTFGQGTKVEIK | |
| 3C8.gr.5.SG | EVQLVQSGAEVKKPGASVKVSCKASGYAFTNYLIEWVRQAPGQGL | 148 |
| V_{H} | EWIGVINPGSGDTYYSEKFKGRVTLTADTSTSTAYLELSSLRSEDTA | |
| | VYYCARDRLDYWGQGTLVTVSS | |
| 3C8.gr.5.SG | DIQMTQSPSSLSASVGDRVTITCHASQDISSYIVWYQQKPGKSFKGL | 149 |
| V_{L} | IYHGTNLESGVPSRFSGSGSGTDFTLTISSLQPEDFATYYCVHYAQF | |
| | PYTFGQGTKVEIK | |
| 3C8.gr.5.EG | EVQLVQSGAEVKKPGASVKVSCKASGYAFTNYLIEWVRQAPGQGL | 150 |
| V_{H} | EWIGVINPGSGDTYYSEKFKGRVTLTADTSTSTAYLELSSLRSEDTA | |
| | VYYCARDRLDYWGQGTLVTVSS | |
| 3C8.gr.5.EG | DIQMTQSPSSLSASVGDRVTITCHASQDISSYIVWYQQKPGKSFKGL | 151 |
| V_{L} | IYHGTNLEEGVPSRFSGSGSGTDFTLTISSLQPEDFATYYCVHYAQF | |
| | PYTFGQGTKVEIK | |
| 3C8.gr.5.QG | EVQLVQSGAEVKKPGASVKVSCKASGYAFTNYLIEWVRQAPGQGL | 152 |
| V_{H} | EWIGVINPGSGDTYYSEKFKGRVTLTADTSTSTAYLELSSLRSEDTA | |
| | VYYCARDRLDYWGQGTLVTVSS | |
| 3C8.gr.5.QG | DIQMTQSPSSLSASVGDRVTITCHASQDISSYIVWYQQKPGKSFKGL | 153 |
| V_{L} | IYHGTNLEQGVPSRFSGSGSGTDFTLTISSLQPEDFATYYCVHYAQF | |
| | PYTFGQGTKVEIK | |
| 3C8.gr.6 | EVQLVQSGAEVKKPGASVKVSCKASGYAFTNYLIEWVRQAPGQGL | 154 |
| V_{H} | EWIGVINPGSGDTYYSEKFKGRVTITADTSTSTAYLELSSLRSEDTAV | |
| | YYCARDRLDYWGQGTLVTVSS | |
| 3C8.gr.6 | DIQMTQSPSSLSASVGDRVTITCHASQDISSYIVWYQQKPGKSFKGL | 155 |
| V_{L} | IYHGTNLEDGVPSRFSGSGSGADYTLTISSLQPEDFATYYCVHYAQF | |
| | PYTFGQGTKVEIK | |
| 3C8.gr.7 | EVQLVQSGAEVKKPGASVKVSCKASGYAFTNYLIEWVRQAPGQGL | 156 |
| V_{H} | EWIGVINPGSGDTYYSEKFKGRVTLTADTSTSTAYLELSSLRSEDTA | |
| | VYYCARDRLDYWGQGTLVTVSS | |
| 3C8.gr.7 | DIQMTQSPSSLSASVGDRVTITCHASQDISSYIVWYQQKPGKSFKGL | 157 |
| V_{L} | IYHGTNLEDGVPSRFSGSGSGADYTLTISSLQPEDFATYYCVHYAQF | |
| | PYTFGQGTKVEIK | |
| 3C8.gr.8 | EVQLVQSGAEVKKPGASVKVSCKASGYAFTNYLIEWVRQAPGQGL | 158 |
| V_{H} | EWIGVINPGSGDTYYSEKFKGRVTLTRDTSTSTAYLELSSLRSEDTA | |
| | VYYCARDRLDYWGQGTLVTVSS | |
| 3C8.gr.8 | DIQMTQSPSSLSASVGDRVTITCHASQDISSYIVWYQQKPGKSFKGL | 159 |
| V_{L} | IYHGTNLEDGVPSRFSGSGSGTDFTLTISSLQPEDFATYYCVHYAQF | |
| | PYTFGQGTKVEIK | |
| 3C8.gr.9 | EVQLVQSGAEVKKPGASVKVSCKASGYAFTNYLIEWVRQAPGQGL | 160 |
| V_{H} | EWIGVINPGSGDTYYSEKFKGRVTLTRDTSTSTAYLELSSLRSEDTA | |
| | VYYCARDRLDYWGQGTLVTVSS | |
| 3C8.gr.9 | DIQMTQSPSSLSASVGDRVTITCHASQDISSYIVWYQQKPGKSPKLLI | 161 |
| V_{L} | YHGTNLEDGVPSRFSGSGSGTDFTLTISSLQPEDFATYYCVHYAQF | |
| | PYTFGQGTKVEIK | |
| 3C8.gr.10 | EVQLVQSGAEVKKPGASVKVSCKASGYAFTNYLIEWVRQAPGQGL | 162 |
| V_{H} | EWIGVINPGSGDTYYSEKFKGRVTLTRDTSTSTAYLELSSLRSEDTA | |
| | VYYCARDRLDYWGQGTLVTVSS | |
| 3C8.gr.10 | DIQMTQSPSSLSASVGDRVTITCHASQDISSYIVWYQQKPGKAFKLLI | 163 |
| V_{L} | YHGTNLEDGVPSRFSGSGSGTDFTLTISSLQPEDFATYYCVHYAQF | |
| | PYTFGQGTKVEIK | |
| 3C8.gr.11 | EVQLVQSGAEVKKPGASVKVSCKASGYAFTNYLIEWVRQAPGQGL | 164 |
| V_{H} | EWIGVINPGSGDTYYSEKFKGRVTLTRDTSTSTAYLELSSLRSEDTA | |
| | VYYCARDRLDYWGQGTLVTVSS | |
| 3C8.gr.11 | DIQMTQSPSSLSASVGDRVTITCHASQDISSYIVWYQQKPGKAPKGL | 165 |
| V_{L} | IYHGTNLEDGVPSRFSGSGSGTDFTLTISSLQPEDFATYYCVHYAQF | |
| | PYTFGQGTKVEIK | |
| 3C8.A.1 | EVQLVQSGAEVKKPGASVKVSCKASGYAFTNYLIEWVRQAPGQGL | 166 |
| V_{H} | EWIGVINPGSGDTYYSEKFKGRVTLTADTSTSTAYLELSSLRSEDTA | |
| | VYYCARDRLDYWGQGTLVTVSS | |
| 3C8.A.1 | DIQMTQSPSSLSASVGDRVTITCHASQDISSYIVWYQQKPGKSFKGL | 167 |
| V_{L} | IYHGTNLEDGVPSRFSGSGSGTDFTLTISSLQPEDFATYYCAHYAQF | |
| | PYTFGQGTKVEIK | |
| 3C8.A.2 | EVQLVQSGAEVKKPGASVKVSCKASGYAFTNYLIEWVRQAPGQGL | 168 |
| V_{H} | EWIGVINPGSGDTYYSEKFKGRVTLTADTSTSTAYLELSSLRSEDTA | |
| | VYYCARDRLDYWGQGTLVTVSS | |
| 3C8.A.2 | DIQMTQSPSSLSASVGDRVTITCHASQDISSYIVWYQQKPGKSFKGL | 169 |
| V_{L} | IYHGTNLEDGVPSRFSGSGSGTDFTLTISSLQPEDFATYYCVAYAQF | |
| | PYTFGQGTKVEIK | |
| 3C8.A.3 | EVQLVQSGAEVKKPGASVKVSCKASGYAFTNYLIEWVRQAPGQGL | 170 |
| V_{H} | EWIGVINPGSGDTYYSEKFKGRVTLTADTSTSTAYLELSSLRSEDTA | |
| | VYYCARDRLDYWGQGTLVTVSS | |
| 3C8.A.3 | DIQMTQSPSSLSASVGDRVTITCHASQDISSYIVWYQQKPGKSFKGL | 171 |
| V_{L} | IYHGTNLEDGVPSRFSGSGSGTDFTLTISSLQPEDFATYYCVHAAQF | |
| | PYTFGQGTKVEIK | |
| 3C8.A.4 | EVQLVQSGAEVKKPGASVKVSCKASGYAFTNYLIEWVRQAPGQGL | 172 |
| V_{H} | EWIGVINPGSGDTYYSEKFKGRVTLTADTSTSTAYLELSSLRSEDTA | |
| | VYYCARDRLDYWGQGTLVTVSS | |
| 3C8.A.4 | DIQMTQSPSSLSASVGDRVTITCHASQDISSYIVWYQQKPGKSFKGL | 173 |
| V_{L} | IYHGTNLEDGVPSRFSGSGSGTDFTLTISSLQPEDFATYYCVHYAAF | |
| | PYTFGQGTKVEIK | |
| 3C8.A.5 | EVQLVQSGAEVKKPGASVKVSCKASGYAFTNYLIEWVRQAPGQGL | 174 |
| V_{H} | EWIGVINPGSGDTYYSEKFKGRVTLTADTSTSTAYLELSSLRSEDTA | |
| | VYYCARDRLDYWGQGTLVTVSS | |
| 3C8.A.5 | DIQMTQSPSSLSASVGDRVTITCHASQDISSYIVWYQQKPGKSFKGL | 175 |
| V_{L} | IYHGTNLEDGVPSRFSGSGSGTDFTLTISSLQPEDFATYYCVHYAQA | |
| | PYTFGQGTKVEIK | |
| 3C8.A.6 | EVQLVQSGAEVKKPGASVKVSCKASGYAFTNYLIEWVRQAPGQGL | 176 |
| V_{H} | EWIGVINPGSGDTYYSEKFKGRVTLTADTSTSTAYLELSSLRSEDTA | |
| | VYYCARDRLDYWGQGTLVTVSS | |
| 3C8.A.6 | DIQMTQSPSSLSASVGDRVTITCHASQDISSYIVWYQQKPGKSFKGL | 177 |
| V_{L} | IYHGTNLEDGVPSRFSGSGSGTDFTLTISSLQPEDFATYYCVHYAQF | |
| | AYTFGQGTKVEIK | |
| 3C8.A.7 | EVQLVQSGAEVKKPGASVKVSCKASGYAFTNYLIEWVRQAPGQGL | 178 |
| V_{H} | EWIGVINPGSGDTYYSEKFKGRVTLTADTSTSTAYLELSSLRSEDTA | |
| | VYYCARDRLDYWGQGTLVTVSS | |
| 3C8.A.7 | DIQMTQSPSSLSASVGDRVTITCHASQDISSYIVWYQQKPGKSFKGL | 179 |
| V_{L} | IYHGTNLEDGVPSRFSGSGSGTDFTLTISSLQPEDFATYYCVHYAQF | |
| | PATFGQGTKVEIK | |
| 3C8.A.8 | EVQLVQSGAEVKKPGASVKVSCKASGYAFTNYLIEWVRQAPGQGL | 180 |
| V_{H} | EWIGVINPGSGDTYYSEKFKGRVTLTADTSTSTAYLELSSLRSEDTA | |
| | VYYCARARLDYWGQGTLVTVSS | |
| 3C8.A.8 | DIQMTQSPSSLSASVGDRVTITCHASQDISSYIVWYQQKPGKSFKGL | 181 |
| V_{L} | IYHGTNLEDGVPSRFSGSGSGTDFTLTISSLQPEDFATYYCVHYAQF | |
| | PYTFGQGTKVEIK | |
| 3C8.A.9 | EVQLVQSGAEVKKPGASVKVSCKASGYAFTNYLIEWVRQAPGQGL | 182 |
| V_{H} | EWIGVINPGSGDTYYSEKFKGRVTLTADTSTSTAYLELSSLRSEDTA | |
| | VYYCARDALDYWGQGTLVTVSS | |
| 3C8.A.9 | DIQMTQSPSSLSASVGDRVTITCHASQDISSYIVWYQQKPGKSFKGL | 183 |
| V_{L} | IYHGTNLEDGVPSRFSGSGSGTDFTLTISSLQPEDFATYYCVHYAQF | |
| | PYTFGQGTKVEIK | |
| 3C8.A.10 | EVQLVQSGAEVKKPGASVKVSCKASGYAFTNYLIEWVRQAPGQGL | 184 |
| V_{H} | EWIGVINPGSGDTYYSEKFKGRVTLTADTSTSTAYLELSSLRSEDTA | |
| | VYYCARDRADYWGQGTLVTVSS | |
| 3C8.A.10 | DIQMTQSPSSLSASVGDRVTITCHASQDISSYIVWYQQKPGKSFKGL | 185 |
| V_{L} | IYHGTNLEDGVPSRFSGSGSGTDFTLTISSLQPEDFATYYCVHYAQF | |
| | PYTFGQGTKVEIK | |
| 1D2.gr.1 | EVQLVESGPGLVKPSETLSLTCTVSGFSLTDYGVLWIRQPPGKGLE | 186 |
| V_{H} | WIGMIWSGGTTDYNAAFISRVTISVDTSKNQFSLKLSSVTAADTAVY | |
| | YCVREEMDYWGQGTLVTVSS | |
| 1D2.gr.1 | DIQMTQSPSSLSASVGDRVTITCRASQDISNFLNWYQQKPGKAPKLL | 187 |
| V_{L} | IYYTSRLHSGVPSRFSGSGSGTDFTLTISSLQPEDFATYYCQQGNTL | |
| | PWTFGQGTKVEIK | |
| 1D2.gr.2 | EVQLVESGPGLVKPSETLSLTCTVSGFSLTDYGVLWIRQPPGKGLE | 188 |
| V_{H} | WIGMIWSGGTTDYNAAFISRVTISKDTSKNQVSLKLSSVTAADTAVY | |
| | YCVREEMDYWGQGTLVTVSS | |
| 1D2.gr.2 | DIQMTQSPSSLSASVGDRVTITCRASQDISNFLNWYQQKPGKAPKLL | 189 |
| V_{L} | IYYTSRLHSGVPSRFSGSGSGTDFTLTISSLQPEDFATYYCQQGNTL | |
| | PWTFGQGTKVEIK | |
| 1D2.gr.3 | EVQLVESGPGLVKPSETLSLTCTVSGFSLTDYGVLWVRQPPGKGLE | 190 |
| V_{H} | WLGMIWSGGTTDYNAAFISRLTISKDTSKNQVSLKLSSVTAADTAVY | |
| | YCVREEMDYWGQGTLVTVSS | |
| 1D2.gr.3 | DIQMTQSPSSLSASVGDRVTITCRASQDISNFLNWYQQKPGKAPKLL | 191 |
| V_{L} | IYYTSRLHSGVPSRFSGSGSGTDFTLTISSLQPEDFATYYCQQGNTL | |
| | PWTFGQGTKVEIK | |
| CON1 | X₁X₂YMS, wherein X₁ is D or E, and X₂ is S or A | 192 |
| (1A7)HVR-H1 | | |
| CON1 (1A7) | DMYPDX₁X₂X₃X₄SYNQKFRE, wherein X₁ is N or S, X₂ is A or G, | 193 |
| HVR-H2 | X₃ is D or S, and X₄ is A or S | |
| CON1 (1A7) | APRWX₁X₂X₃X₄, wherein X₁ is Y or A, X₂ is A or F, X₃ is S | 194 |
| HVR-H3 | or A, and X₄ is A or V. | |
| CON1 (1A7) | QX₁X₂X₃X₄X₅X₆X₇T, wherein X₁ is A or Q, X₂ is A or G, X₃ is | 195 |
| HVR-L3 | A or H, X₄ is A or T, X₅ is A or L, X₆ is A or P, and X₇ | |
| | is A or P. | |
| CON2 (3C8) | VINPGSGDX₁YYSEKFKG, wherein X₁ is T, A or Q. | 196 |
| HVR-H2 | | |
| CON2 (3C8) | HGTNLEX₁, wherein X₁ is S, E, or Q. | 197 |
| HVR-L2 | | |
| CON2 (3C8) | X₁X₂YAQFPYX₃, wherein X₁ is V or A, X₂ is H or A, and X₃ | 198 |
| HVR-L3 | is Y or A. | |

In some embodiments, the OX40 agonist antibody is an anti-human OX40 agonist antibody described in U.S. Pat. No. 7,550,140, which is incorporated herein by reference in its entirety. In some embodiments, the anti-human OX40 agonist antibody comprises a heavy chain comprising the sequence of EVQLVESGGGLVQPGGSLRLSCAASGFTFSNYTMNWVRQAPGKGLEWVSAISGSGGSTYYADSVKGR FTISRDNSKNTLYLQMNSLRAEDTAVYYCAKDRYSQVHYALDYWGQGTLVTVSSASTKGPSVFPLAPSS KSTSGGTAALGCLVKDYFPEPVTVSWNSGALTSGVHTFPAVLQSSGLYSLSSVVTVPSSSLGTQTYICNV NHKPSNTKVDKRVEPKSCDKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYKCKVSNKALPAPIEKTISKAKG QPREPQVYTLPPSREEMTKNQVSLTCLVKGFYPSDIAVEWESNGQPENNYKTTPPVLDSDGSFFLYSKL TVDKSRWQQGNVFSCSVMHEALHNHYTQKSLSLSPGK (SEQ ID NO: 200) and/or a light chain comprising the sequence of DIVMTQSPDSLPVTPGEPASISCRSSQSLLHSNGYNYLDWYLQKAGQSPQLLIYLGSNRASGVPDRFSGS GSGTDFTLKISRVEAEDVGVYYCQQYYNHPTTFGQGTKLEIKRTVAAPSVFIFPPSDEQLKSGTASVVCLL NNFYPREAKVQWKVDNALQSGNSQESVTEQDSKDSTYSLSSTLTLSKADYEKHKVYACEVTHQGLSSP VTKSFNRGEC (SEQ ID NO: 201). In some embodiments, the antibody comprises at least one, two, three, four, five, or six hypervariable region (HVR) sequences of antibody 008 as described in U.S. Pat. No. 7,550,140. In some embodiments, the antibody comprises a heavy chain variable region sequence and/or a light chain variable region sequence of antibody 008 as described in U.S. Pat. No. 7,550,140.

In some embodiments, the OX40 agonist antibody is an anti-human OX40 agonist antibody described in U.S. Pat. No. 7,550,140. In some embodiments, the anti-human OX40 agonist antibody comprises the sequence of DIQMTQSPDSLPVTPGEPASISCRSSQSLLHSNGYNYLDWYLQKAGQSPQLLIYLGSNRASGVPDRFSG SGSGTDFTLKISRVEAEDVGVYYCQQYYNHPTTFGQGTKLEIKRTVAAPSVFIFPPSDEQLKSGTASVVCL LNNFYPREAKVQWKVDNALQSGNSQESVTEQDSKDSTYSLSSTLTLSKADYEKHKVYACEVTHQGLSSP VTKSFNRGEC (SEQ ID NO: 202). In some embodiments, the antibody comprises at least one, two, three, four, five, or six hypervariable region (HVR) sequences of antibody SC02008 as described in U.S. Pat. No. 7,550,140. In some embodiments, the antibody comprises a heavy chain variable region sequence and/or a light chain variable region sequence of antibody SC02008 as described in U.S. Pat. No. 7,550,140.

In some embodiments, the OX40 agonist antibody is an anti-human OX40 agonist antibody described in U.S. Pat. No. 7,550,140. In some embodiments, the anti-human OX40 agonist antibody comprises a heavy chain comprising the sequence of EVQLVESGGGLVHPGGSLRLSCAGSGFTFSSYAMHWVRQAPGKGLEWVSAIGTGGGTYYADSVMGRF TISRDNSKNTLYLQMNSLRAEDTAVYYCARYDNVMGLYWFDYWGQGTLVTVSSASTKGPSVFPLAPSSK STSGGTAALGCLVKDYFPEPVTVSWNSGALTSGVHTFPAVLQSSGLYSLSSVVTVPSSSLGTQTYICNVN HKPSNTKVDKRVEPKSCDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNKALPAPIEKTISKAKGQ PREPQVYTLPPSREEMTKNQVSLTCLVKGFYPSDIAVEWESNGQPENNYKTTPPVLDSDGSFFLYSKLTV DKSRWQQGNVFSCSVMHEALHNHYTQKSLSLSPGK (SEQ ID NO: 203) and/or a light chain comprising the sequence of EIVLTQSPATLSLSPGERATLSCRASQSVSSYLAWYQQKPGQAPRLLIYDASNRATGIPARFSGSGSGTD FTLTISSLEPEDFAVYYCQQRSNWPPAFGGGTKVEIKRTVAAPSVFIFPPSDEQLKSGTASVVCLLNNFYP REAKVQWKVDNALQSGNSQESVTEQDSKDSTYSLSSTLTLSKADYEKHKVYACEVTHQGLSSPVTKSFN RGEC (SEQ ID NO: 204). In some embodiments, the antibody comprises at least one, two, three, four, five, or six hypervariable region (HVR) sequences of antibody 023 as described in U.S. Pat. No. 7,550,140. In some embodiments, the antibody comprises a heavy chain variable region sequence and/or a light chain variable region sequence of antibody 023 as described in U.S. Pat. No. 7,550,140.

In some embodiments, the OX40 agonist antibody is an anti-human OX40 agonist antibody described in U.S. Pat. No. 7,960,515, which is incorporated herein by reference in its entirety. In some embodiments, the anti-human OX40 agonist antibody comprises a heavy chain variable region comprising the sequence of EVQLVESGGGLVQPGGSLRLSCAASGFTFSSYSMNWVRQAPGKGLEWVSYISSSSSTIDYADSVKGRFT ISRDNAKNSLYLQMNSLRDEDTAVYYCARESGWYLFDYWGQGTLVTVSS (SEQ ID NO: 205) and/or a light chain variable region comprising the sequence of DIQMTQSPSSLSASVGDRVTITCRASQGISSWLAWYQQKPEKAPKSLIYAASSLQSGVPSRFSGSGSGT DFTLTISSLQPEDFATYYCQQYNSYPPTFGGGTKVEIK (SEQ ID NO: 206). In some embodiments, the antibody comprises at least one, two, three, four, five, or six hypervariable region (HVR) sequences of antibody 11D4 as described in U.S. Pat. No. 7,960,515. In some embodiments, the antibody comprises a heavy chain variable region sequence and/or a light chain variable region sequence of antibody 11D4 as described in U.S. Pat. No. 7,960,515.

In some embodiments, the OX40 agonist antibody is an anti-human OX40 agonist antibody described in U.S. Pat. No. 7,960,515. In some embodiments, the anti-human OX40 agonist antibody comprises a heavy chain variable region comprising the sequence of EVQLVESGGGLVQPGRSLRLSCAASGFTFDDYAMHWVRQAPGKGLEWVSGISWNSGSIGYADSVKGR FTISRDNAKNSLYLQMNSLRAEDTALYYCAKDQSTADYYFYYGMDVWGQGTTVTVSS (SEQ ID NO: 207) and/or a light chain variable region comprising the sequence of EIVVTQSPATLSLSPGERATLSCRASQSVSSYLAWYQQKPGQAPRLLIYDASNRATGIPARFSGSGSGTD FTLTISSLEPEDFAVYYCQQRSNWPTFGQGTKVEIK (SEQ ID NO: 208). In some embodiments, the antibody comprises at least one, two, three, four, five, or six hypervariable region (HVR) sequences of antibody 18D8 as described in U.S. Pat. No. 7,960,515. In some embodiments, the antibody comprises a heavy chain variable region sequence and/or a light chain variable region sequence of antibody 18D8 as described in U.S. Pat. No. 7,960,515.

In some embodiments, the OX40 agonist antibody is an anti-human OX40 agonist antibody described in WO 2012/027328, which is incorporated herein by reference in its entirety. In some embodiments, the anti-human OX40 agonist antibody comprises a heavy chain variable region comprising the sequence of QVQLVQSGSELKKPGASVKVSCKASGYTFTDYSMHWVRQAPGQGLKWMGWINTETGEPTYADDFKGR FVFSLDTSVSTAYLQISSLKAEDTAVYYCANPYYDYVSYYAMDYWGQGTTVTVSS (SEQ ID NO: 209) and/or a light chain variable region comprising the sequence of DIQMTQSPSSLSASVGDRVTITCKASQDVSTAVAWYQQKPGKAPKLLIYSASYLYTGVPSRFSGSGSGTD FTFTISSLQPEDIATYYCQQHYSTPRTFGQGTKLEIK (SEQ ID NO: 210). In some embodiments, the antibody comprises at least one, two, three, four, five, or six hypervariable region (HVR) sequences of antibody hu106-222 as described in WO 2012/027328. In some embodiments, the antibody comprises a heavy chain variable region sequence and/or a light chain variable region sequence of antibody hu106-222 as described in WO 2012/027328.

In some embodiments, the OX40 agonist antibody is an anti-human OX40 agonist antibody described in WO 2012/027328. In some embodiments, the anti-human OX40 agonist antibody comprises a heavy chain variable region comprising the sequence of EVQLVESGGGLVQPGGSLRLSCAASEYEFPSHDMSWVRQAPGKGLELVAAINSDGGSTYYPDTMERRF TISRDNAKNSLYLQMNSLRAEDTAVYYCARHYDDYYAWFAYWGQGTMVTVSS (SEQ ID NO: 211) and/or a light chain variable region comprising the sequence of EIVLTQSPATLSLSPGERATLSCRASKSVSTSGYSYMHWYQQKPGQAPRLLIYLASNLESGVPARFSGSG SGTDFTLTISSLEPEDFAVYYCQHSRELPLTFGGGTKVEIK (SEQ ID NO: 212). In some embodiments, the antibody comprises at least one, two, three, four, five or six hypervariable region (HVR) sequences of antibody Hu119-122 as described in WO 2012/027328. In some embodiments, the antibody comprises a heavy chain variable region sequence and/or a light chain variable region sequence of antibody Hu119-122 as described in WO 2012/027328.

In some embodiments, the OX40 agonist antibody is an anti-human OX40 agonist antibody described in WO 2013/028231, which is incorporated herein by reference in its entirety. In some embodiments, the anti-human OX40 agonist antibody comprises a heavy chain comprising the sequence of MYLGLNYVFIVFLLNGVQSEVKLEESGGGLVQPGGSMKLSCAASGFTFSDAWMDWVRQSPEKGLEWVA EIRSKANNHATYYAESVNGRFTISRDDSKSSVYLQMNSLRAEDTGIYYCTWGEVFYFDYWGQGTTLTVS SASTKGPSVFPLAPSSKSTSGGTAALGCLVKDYFPEPVTVSWNSGALTSGVHTFPAVLQSSGLYSLSSVV TVPSSSLGTQTYITCNVNHKPSNTKVDKKVEPKSCDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKEYKCKVS NKALPAPIEKTISKAKGQPREPQVYTLPPSRDELTKNQVSLTCLVKGFYPSDIAVEWESNGQPENNYKTT PPVLDSDGSFFLYSKLTVDKSRWQQGNVFSCSVMHEALHNHYTQKSLSLSPGK (SEQ ID NO: 213) and/or a light chain comprising the sequence of MRPSIQFLGLLLFWLHGAQCDIQMTQSPSSLSASLGGKVTITCKSSQDINKYIAWYQHKPGKGPRLLIHYT STLQPGIPSRFSGSGSGRDYSFSISNLEPEDIATYYCLQYDNLLTFGAGTKLELKRTVAAPSVFIFPPSDEQ LKSGTASVVCLLNNFYPREAKVQWKVDNALQSGNSQESVTEQDSKDSTYSLSSTLTLSKADYEKHKVYA CEVTHQGLSSPVTKSFNRGEC (SEQ ID NO: 214). In some embodiments, the antibody comprises at least one, two, three, four, five, or six hypervariable region (HVR) sequences of antibody Mab CH 119-43-1 as described in WO 2013/028231. In some embodiments, the antibody comprises a heavy chain variable region sequence and/or a light chain variable region sequence of antibody Mab CH 119-43-1 as described in WO 2013/028231.

In some embodiments, the OX40 agonist antibody is an anti-human OX40 agonist antibody described in WO 2013/038191, which is incorporated herein by reference in its entirety. In some embodiments, the anti-human OX40 agonist antibody comprises a heavy chain variable region comprising the sequence of EVQLQQSGPELVKPGASVKMSCKASGYTFTSYVMHWVKQKPGQGLEWIGYINPYNDGTKYNEKFKGKA TLTSDKSSSTAYMELSSLTSEDSAVYYCANYYGSSLSMDYWGQGTSVTVSS (SEQ ID NO: 215) and/or a light chain variable region comprising the sequence of DIQMTQTTSSLSASLGDRVTISCRASQDISNYLNWYQQKPDGTVKLLIYYTSRLHSGVPSRFSGSGSGTD YSLTISNLEQEDIATYFCQQGNTLPWTFGGGTKLEIKR (SEQ ID NO: 216). In some embodiments, the antibody comprises at least one, two, three, four, five, or six hypervariable region (HVR) sequences of antibody clone 20E5 as described in WO 2013/038191. In some embodiments, the antibody comprises a heavy chain variable region sequence and/or a light chain variable region sequence of antibody clone 20E5 as described in WO 2013/038191.

In some embodiments, the OX40 agonist antibody is an anti-human OX40 agonist antibody described in WO 2013/038191. In some embodiments, the anti-human OX40 agonist antibody comprises a heavy chain variable region comprising the sequence of EVQLQQSGPELVKPGASVKISCKTSGYTFKDYTMHWVKQSHGKSLEWIGGIYPNNGGSTYNQNFKDKAT LTVDKSSSTAYMEFRSLTSEDSAVYYCARMGYHGPHLDFDVWGAGTTVTVSP (SEQ ID NO: 217) and/or a light chain variable region comprising the sequence of DIVMTQSHKFMSTSLGDRVSITCKASQDVGAAVAWYQQKPGQSPKLLIYWASTRHTGVPDRFTGGGSG TDFTLTISNVQSEDLTDYFCQQYINYPLTFGGGTKLEIKR (SEQ ID NO: 218). In some embodiments, the antibody comprises at least one, two, three, four, five, or six hypervariable region (HVR) sequences of antibody clone 12H3 as described in WO 2013/038191. In some embodiments, the antibody comprises a heavy chain variable region sequence and/or a light chain variable region sequence of antibody clone 12H3 as described in WO 2013/038191.

In some embodiments, the OX40 agonist antibody is an anti-human OX40 agonist antibody described in WO 2014/148895A1, which is incorporated herein by reference in its entirety. In some embodiments, the anti-human OX40 agonist antibody comprises a heavy chain variable region comprising the sequence of QVQLVQSGAEVKKPGASVKVSCKASGYTFTSYVMHWVRQAPGQRLEWMGYINPYNDGTKYNEKFKGR VTITSDTSASTAYMELSSLRSEDTAVYYCANYYGSSLSMDYWGQGTLVTVSS (SEQ ID NO: 219) and/or a light chain variable region comprising the sequence of DIQMTQSPSSLSASVGDRVTITCRASQDISNYLNWYQQKPGKAPKLLIYYTSRLHSGVPSRFSGSGSGTD YTLTISSLQPEDFATYYCQQGNTLPWTFGQGTKVEIKR (SEQ ID NO: 220). In some embodiments, the antibody comprises at least one, two, three, four, five, or six hypervariable region (HVR) sequences of antibody clone 20E5 as described in WO 2014/148895A1. In some embodiments, the antibody comprises a heavy chain variable region sequence and/or a light chain variable region sequence of antibody clone 20E5 as described in WO 2014/148895A1.

In some embodiments, the OX40 agonist antibody is an anti-human OX40 agonist antibody described in WO 2014/148895A1. In some embodiments, the anti-human OX40 agonist antibody comprises a heavy chain variable region comprising the sequence of QVQLVQSGAEVKKPGASVKVSCKASGYTFTSYVMHWVRQAPGQRLEWMGYINPYNDGTKYNEKFKGR VTITSDTSASTAYMELSSLRSEDTAVYYCANYYGSSLSMDYWGQGTLVTVSS (SEQ ID NO: 219) and/or a light chain variable region comprising the sequence of DIQMTQSPSSLSASVGDRVTITCRASQDISNYLNWYQQKPGKAVKLLIYYTSRLHSGVPSRFSGSGSGTD YTLTISSLQPEDFATYFCQQGNTLPWTFGQGTKVEIKR (SEQ ID NO: 221). In some embodiments, the antibody comprises at least one, two, three, four, five, or six hypervariable region (HVR) sequences of antibody clone 20E5 as described in WO 2014/148895A1. In some embodiments, the antibody comprises a heavy chain variable region sequence and/or a light chain variable region sequence of antibody clone 20E5 as described in WO 2014/148895A1.

In some embodiments the OX40 agonist antibody is an anti-human OX40 agonist antibody described in WO 2014/148895A1. In some embodiments, the anti-human OX40 agonist antibody comprises a heavy chain variable region comprising the sequence of QVQLVQSGAEVKKPGASVKVSCKASGYTFTSYVMHWVRQAPGQRLEWIGYINPYNDGTKYNEKFKGRA TITSDTSASTAYMELSSLRSEDTAVYYCANYYGSSLSMDYWGQGTLVTVSS (SEQ ID NO: 222) and/or a light chain variable region comprising the sequence of DIQMTQSPSSLSASVGDRVTITCRASQDISNYLNWYQQKPGKAPKLLIYYTSRLHSGVPSRFSGSGSGTD YTLTISSLQPEDFATYYCQQGNTLPWTFGQGTKVEIKR (SEQ ID NO: 220). In some embodiments, the antibody comprises at least one, two, three, four, five, or six hypervariable region (HVR) sequences of antibody clone 20E5 as described in WO 2014/148895A1. In some embodiments, the antibody comprises a heavy chain variable region sequence and/or a light chain variable region sequence of antibody clone 20E5 as described in WO 2014/148895A1.

In some embodiments, the OX40 agonist antibody is an anti-human OX40 agonist antibody described in WO 2014/148895A1. In some embodiments, the anti-human OX40 agonist antibody comprises a heavy chain variable region comprising the sequence of QVQLVQSGAEVKKPGASVKVSCKASGYTFTSYVMHWVRQAPGQRLEWIGYINPYNDGTKYNEKFKGRA TITSDTSASTAYMELSSLRSEDTAVYYCANYYGSSLSMDYWGQGTLVTVSS (SEQ ID NO: 222) and/or a light chain variable region comprising the sequence of DIQMTQSPSSLSASVGDRVTITCRASQDISNYLNWYQQKPGKAVKLLIYYTSRLHSGVPSRFSGSGSGTD YTLTISSLQPEDFATYFCQQGNTLPWTFGQGTKVEIKR (SEQ ID NO: 221). In some embodiments, the antibody comprises at least one, two, three, four, five, or six hypervariable region (HVR) sequences of antibody clone 20E5 as described in WO 2014/148895A1. In some embodiments, the antibody comprises a heavy chain variable region sequence and/or a light chain variable region sequence of antibody clone 20E5 as described in WO 2014/148895A1.

In some embodiments, the OX40 agonist antibody is an anti-human OX40 agonist antibody described in WO 2014/148895A1. In some embodiments, the anti-human OX40 agonist antibody comprises a heavy chain variable region comprising the sequence of QVQLVQSGAEVKKPGASVKVSCKASGYTFTSYVMHWVRQAPGQRLEWIGYINPYNDGTKYNEKFKGRA TLTSDKSASTAYMELSSLRSEDTAVYYCANYYGSSLSMDYWGQGTLVTVSS (SEQ ID NO: 223) and/or a light chain variable region comprising the sequence of DIQMTQSPSSLSASVGDRVTITCRASQDISNYLNWYQQKPGKAPKLLIYYTSRLHSGVPSRFSGSGSGTD YTLTISSLQPEDFATYYCQQGNTLPWTFGQGTKVEIKR (SEQ ID NO: 220). In some embodiments, the antibody comprises at least one, two, three, four, five, or six hypervariable region (HVR) sequences of antibody clone 20E5 as described in WO 2014/148895A1. In some embodiments, the antibody comprises a heavy chain variable region sequence and/or a light chain variable region sequence of antibody clone 20E5 as described in WO 2014/148895A1.

In some embodiments, the OX40 agonist antibody is an anti-human OX40 agonist antibody described in WO 2014/148895A1. In some embodiments, the anti-human OX40 agonist antibody comprises a heavy chain variable region comprising the sequence of QVQLVQSGAEVKKPGASVKVSCKASGYTFTSYVMHWVRQAPGQRLEWIGYINPYNDGTKYNEKFKGRA TLTSDKSASTAYMELSSLRSEDTAVYYCANYYGSSLSMDYWGQGTLVTVSS (SEQ ID NO: 223) and/or a light chain variable region comprising the sequence of DIQMTQSPSSLSASVGDRVTITCRASQDISNYLNWYQQKPGKAVKLLIYYTSRLHSGVPSRFSGSGSGTD YTLTISSLQPEDFATYFCQQGNTLPWTFGQGTKVEIKR (SEQ ID NO: 221). In some embodiments, the antibody comprises at least one, two, three, four, five, or six hypervariable region (HVR) sequences of antibody clone 20E5 as described in WO 2014/148895A1. In some embodiments, the antibody comprises a heavy chain variable region sequence and/or a light chain variable region sequence of antibody clone 20E5 as described in WO 2014/148895A1.

In some embodiments, the OX40 agonist antibody is an anti-human OX40 agonist antibody described in WO 2014/148895A1. In some embodiments, the anti-human OX40 agonist antibody comprises a heavy chain variable region comprising the sequence of QVQLVQSGAEVKKPGSSVKVSCKASGYTFKDYTMHWVRQAPGQGLEWMGGIYPNNGGSTYNQNFKD RVTITADKSTSTAYMELSSLRSEDTAVYYCARMGYHGPHLDFDVWGQGTTVTVSS (SEQ ID NO: 224) and/or a light chain variable region comprising the sequence of DIQMTQSPSSLSASVGDRVTITCKASQDVGAAVAWYQQKPGKAPKLLIYWASTRHTGVPSRFSGSGSGT DFTLTISSLQPEDFATYYCQQYINYPLTFGGGTKVEIKR (SEQ ID NO: 225). In some embodiments, the antibody comprises at least one, two, three, four, five, or six hypervariable region (HVR) sequences of antibody clone clone 12H3 as described in WO 2014/148895A1. In some embodiments, the antibody comprises a heavy chain variable region sequence and/or a light chain variable region sequence of antibody clone 12H3 as described in WO 2014/148895A1.

In some embodiments, the OX40 agonist antibody is an anti-human OX40 agonist antibody described in WO 2014/148895A1. In some embodiments, the anti-human OX40 agonist antibody comprises a heavy chain variable region comprising the sequence of QVQLVQSGAEVKKPGSSVKVSCKASGYTFKDYTMHWVRQAPGQGLEWMGGIYPNNGGSTYNQNFKD RVTITADKSTSTAYMELSSLRSEDTAVYYCARMGYHGPHLDFDVWGQGTTVTVSS (SEQ ID NO: 224) and/or a light chain variable region comprising the sequence of DIQMTQSPSSLSASVGDRVTITCKASQDVGAAVAWYQQKPGKAPKLLIYWASTRHTGVPDRFSGGGSGT DFTLTISSLQPEDFATYYCQQYINYPLTFGGGTKVEIKR (SEQ ID NO: 226). In some embodiments, the antibody comprises at least one, two, three, four, five, or six hypervariable region (HVR) sequences of antibody clone 12H3 as described in WO 2014/148895A1. In some embodiments, the antibody comprises a heavy chain variable region sequence and/or a light chain variable region sequence of antibody clone 12H3 as described in WO 2014/148895A1.

In some embodiments, the OX40 agonist antibody is an anti-human OX40 agonist antibody described in WO 2014/148895A1. In some embodiments, the anti-human OX40 agonist antibody comprises a heavy chain variable region comprising the sequence of QVQLVQSGAEVKKPGSSVKVSCKASGYTFKDYTMHWVRQAPGQGLEWIGGIYPNNGGSTYNQNFKDR VTLTADKSTSTAYMELSSLRSEDTAVYYCARMGYHGPHLDFDVWGQGTTVTVSS (SEQ ID NO: 227) and/or a light chain variable region comprising the sequence of DIQMTQSPSSLSASVGDRVTITCKASQDVGAAVAWYQQKPGKAPKLLIYWASTRHTGVPSRFSGSGSGT DFTLTISSLQPEDFATYYCQQYINYPLTFGGGTKVEIKR (SEQ ID NO: 225). In some embodiments, the antibody comprises at least one, two, three, four, five, or six hypervariable region (HVR) sequences of antibody clone 12H3 as described in WO 2014/148895A1. In some embodiments, the antibody comprises a heavy chain variable region sequence and/or a light chain variable region sequence of antibody clone 12H3 as described in WO 2014/148895A1.

In some embodiments, the OX40 agonist antibody is an anti-human OX40 agonist antibody described in WO 2014/148895A1. In some embodiments, the anti-human OX40 agonist antibody comprises a heavy chain variable region comprising the sequence of QVQLVQSGAEVKKPGSSVKVSCKASGYTFKDYTMHWVRQAPGQGLEWIGGIYPNNGGSTYNQNFKDR VTLTADKSTSTAYMELSSLRSEDTAVYYCARMGYHGPHLDFDVWGQGTTVTVSS (SEQ ID NO: 227) and/or a light chain variable region comprising the sequence of DIQMTQSPSSLSASVGDRVTITCKASQDVGAAVAWYQQKPGKAPKLLIYWASTRHTGVPDRFSGGGSGT DFTLTISSLQPEDFATYYCQQYINYPLTFGGGTKVEIKR (SEQ ID NO: 226). In some embodiments, the antibody comprises at least one, two, three, four, five, or six hypervariable region (HVR) sequences of antibody clone 12H3 as described in WO 2014/148895A1. In some embodiments, the antibody comprises a heavy chain variable region sequence and/or a light chain variable region sequence of antibody clone 12H3 as described in WO 2014/148895A1.

In some embodiments, the OX40 agonist antibody is an anti-human OX40 agonist antibody described in WO 2014/148895A1. In some embodiments, the anti-human OX40 agonist antibody comprises a heavy chain variable region comprising the sequence of QVQLVQSGAEVKKPGSSVKVSCKASGYTFKDYTMHWVRQAPGQGLEWIGGIYPNNGGSTYNQNFKDR ATLTVDKSTSTAYMELSSLRSEDTAVYYCARMGYHGPHLDFDVWGQGTTVTVSS (SEQ ID NO: 228) and/or a light chain variable region comprising the sequence of DIQMTQSPSSLSASVGDRVTITCKASQDVGAAVAWYQQKPGKAPKLLIYWASTRHTGVPSRFSGSGSGT DFTLTISSLQPEDFATYYCQQYINYPLTFGGGTKVEIKR (SEQ ID NO: 225). In some embodiments, the antibody comprises at least one, two, three, four, five, or six hypervariable region (HVR) sequences of antibody clone 12H3 as described in WO 2014/148895A1. In some embodiments, the antibody comprises a heavy chain variable region sequence and/or a light chain variable region sequence of antibody clone 12H3 as described in WO 2014/148895A1.

In some embodiments, the OX40 agonist antibody is an anti-human OX40 agonist antibody described in WO 2014/148895A1. In some embodiments, the anti-human OX40 agonist antibody comprises a heavy chain variable region comprising the sequence of QVQLVQSGAEVKKPGSSVKVSCKASGYTFKDYTMHWVRQAPGQGLEWIGGIYPNNGGSTYNQNFKDR ATLTVDKSTSTAYMELSSLRSEDTAVYYCARMGYHGPHLDFDVWGQGTTVTVSS (SEQ ID NO: 228) and/or a light chain variable region comprising the sequence of DIQMTQSPSSLSASVGDRVTITCKASQDVGAAVAWYQQKPGKAPKLLIYWASTRHTGVPDRFSGGGSGT DFTLTISSLQPEDFATYYCQQYINYPLTFGGGTKVEIKR (SEQ ID NO: 226). In some embodiments, the antibody comprises at least one, two, three, four, five, or six hypervariable region (HVR) sequences of antibody clone 12H3 as described in WO 2014/148895A1. In some embodiments, the antibody comprises a heavy chain variable region sequence and/or a light chain variable region sequence of antibody clone 12H3 as described in WO 2014/148895A1.

In some embodiments, the OX40 agonist antibody is L106 BD (Pharmingen Product #340420). In some embodiments, the antibody comprises at least one, two, three, four, five, or six hypervariable region (HVR) sequences of antibody L106 (BD Pharmingen Product #340420). In some embodiments, the antibody comprises a heavy chain variable region sequence and/or a light chain variable region sequence of antibody L106 (BD Pharmingen Product #340420).

In some embodiments the OX40 agonist antibody is ACT35 (Santa Cruz Biotechnology, Catalog #20073). In some embodiments, the antibody comprises at least one, two, three, four, five, or six hypervariable region (HVR) sequences of antibody ACT35 (Santa Cruz Biotechnology, Catalog #20073). In some embodiments, the antibody comprises a heavy chain variable region sequence and/or a light chain variable region sequence of antibody ACT35 (Santa Cruz Biotechnology, Catalog #20073).

In some embodiments, the OX40 agonist antibody is MEDI6469. In some embodiments, the antibody comprises at least one, two, three, four, five, or six hypervariable region (HVR) sequences of antibody MEDI6469. In some embodiments, the antibody comprises a heavy chain variable region sequence and/or a light chain variable region sequence of antibody MEDI6469.

In some embodiments, the OX40 agonist antibody is MEDI0562. In some embodiments, the antibody comprises at least one, two, three, four, five, or six hypervariable region (HVR) sequences of antibody MEDI0562. In some embodiments, the antibody comprises a heavy chain variable region sequence and/or a light chain variable region sequence of antibody MEDI0562.

In some embodiments, the OX40 agonist antibody is an agonist antibody that binds to the same epitope as any one of the OX40 agonist antibodies set forth above.

OX40 agonists useful for the methods described herein are in no way intended to be limited to antibodies. Non-antibody OX40 agonists are contemplated and well known in the art. As described above, OX40L (also known as CD134L) serves as a ligand for OX40. As such, agonists that present part or all of OX40L may serve as OX40 agonists. In some embodiments, an OX40 agonist may include one or more extracellular domains of OX40L. Examples of extracellular domains of OX40L may include OX40-binding domains. In some embodiments, an OX40 agonist may be a soluble form of OX40L that includes one or more extracellular domains of OX40L but lacks other, insoluble domains of the protein, e.g., transmembrane domains. In some embodiments, an OX40 agonist is a soluble protein that includes one or more extracellular domains of OX40L able to bind OX40L. In some embodiments, an OX40 agonist may be linked to another protein domain, e.g., to increase its effectiveness, half-life, or other desired characteristics. In some embodiments, an OX40 agonist may include one or more extracellular domains of OX40L linked to an immunoglobulin Fc domain.

In some embodiments, an OX40 agonist may be an oligomeric or multimeric molecule. For example, an OX40 agonist may contain one or more domains (e.g., a leucine zipper domain) that allows proteins to oligomerize. In some embodiments, an OX40 agonist may include one or more extracellular domains of OX40L linked to one or more leucine zipper domains.

In some embodiments, an OX40 agonist may be any one of the OX40 agonists described in European Patent No. EP0672141 B1.

In some embodiments, an OX40 agonist may be a trimeric OX40L fusion protein. For example, an OX40 agonist may include one or more extracellular domains of OX40L linked to an immunoglobulin Fc domain and a trimerization domain (including without limitation an isoleucine zipper domain).

In some embodiments, an OX40 agonist may be any one of the OX40 agonists described in International Publication No. WO2006/121810, such as an OX40 immunoadhesin. In some embodiments, the OX40 immunoadhesin may be a trimeric OX40-Fc protein. In some embodiments, the OX40 agonist is MEDI6383.

B. Agents that Decrease or Inhibit TIGIT Expression and/or TIGIT Activity

Provided herein is a method for treatment or delaying progression of cancer in an individual comprising administering to the individual an effective amount of an OX40 binding agonist in combination with an agent that decreases or inhibits TIGIT expression and/or activity. Provided herein is also a method for reducing or inhibiting cancer relapse or cancer progression in an individual comprising administering to the individual an effective amount of an OX40 binding agonist in combination with an agent that decreases or inhibits TIGIT expression and/or activity. Provided herein is also a method for treating or delaying progression of an immune related disease in an individual comprising administering to the individual an effective amount of an OX40 binding agonist in combination with an agent that decreases or inhibits TIGIT expression and/or activity. Provided herein is also a method for reducing or inhibiting progression of an immune related disease in an individual comprising administering to the individual an effective amount of an OX40 binding agonist in combination with an agent that decreases or inhibits TIGIT expression and/or activity. Provided herein is also a method for increasing, enhancing, or stimulating an immune response or function in an individual comprising administering to the individual an effective amount of an OX40 binding agonist in combination with an agent that decreases or inhibits TIGIT expression and/or activity.

Provided herein is also a method for increasing, enhancing, or stimulating an immune response or function in an individual comprising administering to the individual an effective amount of an OX40 binding agonist in combination an effective amount of an agent that decreases or inhibits TIGIT expression and/or activity and an agent that decreases or inhibits one or more additional immune co-inhibitory receptors. Provided herein is also a method for increasing, enhancing, or stimulating an immune response or function in an individual comprising administering to the individual an effective amount of an OX40 binding agonist in combination an effective amount of an agent that decreases or inhibits TIGIT expression and/or activity and an agent that increases or activates one or more additional immune co-stimulatory receptors.

An agent that decreases or inhibits TIGIT expression and/or TIGIT activity includes, for example, an antagonist of TIGIT expression and/or activity, an antagonist of PVR expression and/or activity, an agent that inhibits and/or blocks the interaction of TIGIT with PVR, an agent that inhibits and/or blocks the interaction of TIGIT with PVRL2, an agent that inhibits and/or blocks the interaction of TIGIT with PVRL3, an agent that inhibits and/or blocks the intracellular signaling mediated by TIGIT binding to PVR, an agent that inhibits and/or blocks the intracellular signaling mediated by TIGIT binding to PVRL2, an agent that inhibits and/or blocks the intracellular signaling mediated by TIGIT binding to PVRL3, and combinations thereof.

In some embodiments, the antagonist of TIGIT expression and/or activity includes a small molecule inhibitor, an inhibitory antibody or antigen-binding fragment thereof, an aptamer, an inhibitory nucleic acid, and an inhibitory polypeptide.

In some embodiments, the antagonist of PVR expression and/or activity includes a small molecule inhibitor, an inhibitory antibody or antigen-binding fragment thereof, an aptamer, an inhibitory nucleic acid, and an inhibitory polypeptide.

In some embodiments, the agent that inhibits and/or blocks the interaction of TIGIT with PVR includes a small molecule inhibitor, an inhibitory antibody or antigen-binding fragment thereof, an aptamer, an inhibitory nucleic acid, and an inhibitory polypeptide.

In some embodiments, the agent that inhibits and/or blocks the interaction of TIGIT with PVRL2 includes a small molecule inhibitor, an inhibitory antibody or antigen-binding fragment thereof, an aptamer, an inhibitory nucleic acid, and an inhibitory polypeptide.

In some embodiments, the agent that inhibits and/or blocks the interaction of TIGIT with PVRL3 includes a small molecule inhibitor, an inhibitory antibody or antigen-binding fragment thereof, an aptamer, an inhibitory nucleic acid, and an inhibitory polypeptide.

In some embodiments, the agent that inhibits and/or blocks the intracellular signaling mediated by TIGIT binding to PVR includes a small molecule inhibitor, an inhibitory antibody or antigen-binding fragment thereof, an aptamer, an inhibitory nucleic acid, and an inhibitory polypeptide.

In some embodiments, the agent that inhibits and/or blocks the intracellular signaling mediated by TIGIT binding to PVRL2 includes a small molecule inhibitor, an inhibitory antibody or antigen-binding fragment thereof, an aptamer, an inhibitory nucleic acid, and an inhibitory polypeptide.

In some embodiments, the agent that inhibits and/or blocks the intracellular signaling mediated by TIGIT binding to PVRL3 includes a small molecule inhibitor, an inhibitory antibody or antigen-binding fragment thereof, an aptamer, an inhibitory nucleic acid, and an inhibitory polypeptide.

In some embodiments, the antagonist of TIGIT expression and/or activity is an inhibitory nucleic acid selected from an antisense polynucleotide, an interfering RNA, a catalytic RNA, and an RNA-DNA chimera.

In some embodiments, the antagonist of TIGIT expression and/or activity is an anti-TIGIT antibody, or antigen-binding fragment thereof.

The anti-TIGIT antibodies useful in this invention, including compositions containing such antibodies, such as those described in WO 2009/126688, may be used in combination with one or more OX40 binding agonists, such as those described above.

The present invention provides anti-TIGIT antibodies. Exemplary anti-TIGIT antibodies include polyclonal, monoclonal, humanized, bispecific, and heteroconjugate antibodies, or antibody fragments (e.g., antigen-binding fragments) thereof. In another embodiment, the anti-TIGIT antibody is a full-length antibody, e.g., an intact IgG antibody (e.g., an intact IgG1 antibody) or other antibody class or isotype as defined herein. It will be understood by one of ordinary skill in the art that the invention also provides antibodies against other polypeptides (i.e., anti-PVR antibodies) and that any of the description herein drawn specifically to the method of creation, production, varieties, use or other aspects of anti-TIGIT antibodies will also be applicable to antibodies specific for other non-TIGIT polypeptides.

In some embodiments, anti-TIGIT antibodies were generated which were hamster-anti-mouse antibodies. Two such antibodies, 10A7 and 1F4, bound specifically to human TIGIT. The amino acid sequences of the light and heavy chains of the 10A7 antibody were determined using standard techniques. The light chain sequence of this antibody is: DIVMTQSPSSLAVSPGEKVTMTCKSSQSLYYSGVKENLLAWYQQKPGQSPKLLIYYASIRFTGVPDRFTG SGSGTDYTLTITSVQAEDMGQYFCQQGINNPLTFGDGTKLEIKR (SEQ ID NO:13) and the heavy chain sequence of this antibody is: EVQLVESGGGLTQPGKSLKLSCEASGFTFSSFTMHWVRQSPGKGLEWVAFIRSGSGIVFYADAVRGRFT ISRDNAKNLLFLQMNDLKSEDTAMYYCARRPLGHNTFDSWGQGTLVTVSS (SEQ ID NO:15), where the complementarity determining regions (CDRs) of each chain are represented by bold text. Thus, HVR1 of the 10A7 light chain has the sequence KSSQSLYYSGVKENLLA (SEQ ID NO:1), HVR2 of the 10A7 light chain has the sequence ASIRFT (SEQ ID NO:2), and HVR3 of the 10A7 light chain has the sequence QQGINNPLT (SEQ ID NO:3). HVR1 of the 10A7 heavy chain has the sequence GFTFSSFTMH (SEQ ID NO:4), HVR2 of the 10A7 heavy chain has the sequence FIRSGSGIVFYADAVRG (SEQ ID NO:5), and HVR3 of the 10A7 heavy chain has the sequence RPLGHNTFDS (SEQ ID NO:6).

The amino acid sequences of the light and heavy chains of the 1F4 antibody were also determined. The light chain sequence of this antibody is: DVVLTQTPLSLSVSFGDQVSISCRSSQSLVNSYGNTFLSWYLHKPGQSPQLLIFGISNRFSGVPDRFSGS GSGTDFTLKISTIKPEDLGMYYCLQGTHQPPTFGPGTKLEVK (SEQ ID NO:14) and the heavy chain sequence of this antibody is: EVQLQQSGPELVKPGTSMKISCKASGYSFTGHLMNWVKQSHGKNLEWIGLIIPYNGGTSYNQKFKGKAT LTVDKSSSTAYMELLSLTSDDSAVYFCSRGLRGFYAMDYWGQGTSVTVSS (SEQ ID NO:16), where the complementarity determining regions (HVRs) of each chain are represented by bold text. Thus, HVR1 of the 1F4 light chain has the sequence RSSQSLVNSYGNTFLS (SEQ ID NO:7), HVR2 of the 1F4 light chain has the sequence GISNRFS (SEQ ID NO:8), and HVR3 of the 1F4 light chain has the sequence LQGTHQPPT (SEQ ID NO:9). HVR1 of the 1F4 heavy chain has the sequence GYSFTGHLMN (SEQ ID NO:10), HVR2 of the 1F4 heavy chain has the sequence LIIPYNGGTSYNQKFKG (SEQ ID NO:11), and HVR3 of the 1F4 heavy chain has the sequence GLRGFYAMDY (SEQ ID NO:12).

In some embodiments, the anti-TIGIT antibody, or antigen-binding fragment thereof, comprises at least one HVR (e.g., one, two, three, four, five, or all six HVRs) comprising an amino acid sequence selected from the amino acid sequences set forth in KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), RPLGHNTFDS (SEQ ID NO:6), RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12).

In some embodiments, the anti-TIGIT antibody, or antigen-binding fragment thereof, comprises a light chain comprising the amino acid sequence set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGTKLEIKR (SEQ ID NO:13) or DVVLTQTPLSLSVSFGDQVSISCRSSQSLVNSYGNTFLSWYLHKPGQSPQLLIFGISNRFSGVPDRFSGS GSGTDFTLKISTIKPEDLGMYYCLQGTHQPPTFGPGTKLEVK (SEQ ID NO:14).

In some embodiments, the anti-TIGIT antibody, or antigen-binding fragment thereof, comprises a heavy chain comprising the amino acid sequence set forth in EVQLVESGGGLTQPGKSLKLSCEASGFTFSSFTMHWVRQSPGKGLEWVAFIRSGSGIVFYADAVRGRFT ISRDNAKNLLFLQMNDLKSEDTAMYYCARRPLGHNTFDSWGQGTLVTVSS (SEQ ID NO:15) or EVQLQQSGPELVKPGTSMKISCKASGYSFTGHLMNWVKQSHGKNLEWIGLIIPYNGGTSYNQKFKGKAT LTVDKSSSTAYMELLSLTSDDSAVYFCSRGLRGFYAMDYWGQGTSVTVSS (SEQ ID NO:16).

In some embodiments, the anti-TIGIT antibody, or antigen-binding fragment thereof, comprises a light chain comprising the amino acid sequence set forth in DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGTKLEIKR (SEQ ID NO:13) or DVVLTQTPLSLSVSFGDQVSISCRSSQSLVNSYGNTFLSWYLHKPGQSPQLLIFGISNRFSGVPDRFSGS GSGTDFTLKISTIKPEDLGMYYCLQGTHQPPTFGPGTKLEVK (SEQ ID NO:14), and a heavy chain comprising the amino acid sequence set forth in EVQLVESGGGLTQPGKSLKLSCEASGFTFSSFTMHWVRQSPGKGLEWVAFIRSGSGIVFYADAVRGRFT ISRDNAKNLLFLQMNDLKSEDTAMYYCARRPLGHNTFDSWGQGTLVTVSS (SEQ ID NO:15) or EVQLQQSGPELVKPGTSMKISCKASGYSFTGHLMNWVKQSHGKNLEWIGLIIPYNGGTSYNQKFKGKAT LTVDKSSSTAYMELLSLTSDDSAVYFCSRGLRGFYAMDYWGQGTSVTVSS (SEQ ID NO:16).

In some embodiments, the anti-TIGIT antibody, or antigen-binding fragment thereof, is selected from a humanized antibody, a chimeric antibody, a bispecific antibody, a heteroconjugate antibody, and an immunotoxin.

In some embodiments, the anti-TIGIT antibody, or antigen-binding fragment thereof, comprises at least one HVR (e.g., one, two, three, four, five, or all six HVRs) having at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to, or the sequence of, KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), RPLGHNTFDS (SEQ ID NO:6), RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and/or GLRGFYAMDY (SEQ ID NO:12).

In some embodiments, the anti-TIGIT antibody, or fragment thereof, comprises a light chain having at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to, or the sequence of, DIVMTQSPSSLAVSPGEKVTMTCKSSQSLYYSGVKENLLAWYQQKPGQS PKLLIYYASIRFTGVPDRFTGSGSGTDYTLTITSVQAEDMGQYFCQQGINNPLTFGDGTKLEIKR (SEQ ID NO:13) or DVVLTQTPLSLSVSFGDQVSISCRSSQSLVNSYGNTFLSWYLHKPGQSPQLLIFGISNRFSGVPDRFSGS GSGTDFTLKISTIKPEDLGMYYCLQGTHQPPTFGPGTKLEVK (SEQ ID NO:14), and/or a heavy chain having at least 80% sequence identity (e.g., at least 80%, 81%, 82%, 83%, 84%, 85%, 86%, 87%, 88%, 89%, 90%, 91%, 92%, 93%, 94%, 95%, 96%, 97%, 98%, or 99% sequence identity) to, or the sequence of, EVQLVESGGGLTQPGKSLKLSCEASGFTFSSFTMHWVRQSPGKGLEWVAFIRSGSGIVFYADAVRGRFT ISRDNAKNLLFLQMNDLKSEDTAMYYCARRPLGHNTFDSWGQGTLVTVSS (SEQ ID NO:15) or EVQLQQSGPELVKPGTSMKISCKASGYSFTGHLMNWVKQSHGKNLEWIGLIIPYNGGTSYNQKFKGKAT LTVDKSSSTAYMELLSLTSDDSAVYFCSRGLRGFYAMDYWGQGTSVTVSS (SEQ ID NO:16).

In some embodiments, the anti-TIGIT antibody, or antigen-binding fragment thereof, binds to the same epitope as an antibody comprising one of the following sets of six HVR sequences: (a) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (b) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12).

C. Agents that Modulate CD226 Expression and/or Activity

Provided herein is a method of treating or delaying progression of cancer in an individual comprising administering to the individual an effective amount of an OX40 binding agonist and an agent that modulates the CD226 expression and/or activity. Provided herein is also a method for reducing or inhibiting cancer relapse or cancer progression in an individual comprising administering to the individual an effective amount of an OX40 binding agonist and an agent that modulates the CD226 expression and/or activity. Provided herein is also a method for treating or delaying progression of an immune related disease in an individual comprising administering to the individual an effective amount of an OX40 binding agonist and an agent that modulates the CD226 expression and/or activity. Provided herein is also a method for reducing or inhibiting progression of an immune related disease in an individual comprising administering to the individual an effective amount of an OX40 binding agonist and agent that modulates the CD226 expression and/or activity. Provided herein is also a method of increasing, enhancing or stimulating an immune response or function in an individual by administering to the individual an effective amount of an OX40 binding agonist and an agent that modulates the CD226 expression and/or activity.

For example, agents that modulate the CD226 expression and/or activity are agents capable of increasing and/or stimulating CD226 expression and/or activity, increasing and/or stimulating the interaction of CD226 with PVR, PVRL2, and/or PVRL3, and increasing and/or stimulating the intracellular signaling mediated by CD226 binding to PVR, PVRL2, and/or PVRL3. In some embodiments, agents capable of increasing and/or stimulating CD226 expression and/or activity are agents that increase and/or stimulate CD226 expression and/or activity. In some embodiments, agents capable of increasing and/or stimulating the interaction of CD226 with PVR, PVRL2, and/or PVRL3 are agents that increase and/or stimulate the interaction of CD226 with PVR, PVRL2, and/or PVRL3. In some embodiments, agents capable of increasing and/or stimulating the intracellular signaling mediated by CD226 binding to PVR, PVRL2, and/or PVRL3 are agents that increase and/or stimulate the intracellular signaling mediated by CD226 binding to PVR, PVRL2, and/or PVRL3.

In some embodiments, the agent that modulates the CD226 expression and/or activity is selected from an agent that inhibits and/or blocks the interaction of CD226 with TIGIT, an antagonist of TIGIT expression and/or activity, an antagonist of PVR expression and/or activity, an agent that inhibits and/or blocks the interaction of TIGIT with PVR, an agent that inhibits and/or blocks the interaction of TIGIT with PVRL2, an agent that inhibits and/or blocks the interaction of TIGIT with PVRL3, an agent that inhibits and/or blocks the intracellular signaling mediated by TIGIT binding to PVR, an agent that inhibits and/or blocks the intracellular signaling mediated by TIGIT binding to PVRL2, an agent that inhibits and/or blocks the intracellular signaling mediated by TIGIT binding to PVRL3, and combinations thereof. In some embodiments, the agent that inhibits and/or blocks the interaction of CD226 with TIGIT is selected from a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of CD226 with TIGIT is an anti-TIGIT antibody or antigen-binding fragment thereof. In some embodiments, the agent that inhibits and/or blocks the interaction of CD226 with TIGIT is an inhibitory nucleic acid selected from an antisense polynucleotide, an interfering RNA, a catalytic RNA, and an RNA-DNA chimera.

In some embodiments, the antagonist of TIGIT expression and/or activity is a small molecule inhibitor, an inhibitory antibody or antigen-binding fragment thereof, an aptamer, an inhibitory nucleic acid, and an inhibitory polypeptide. In some embodiments, the antagonist of TIGIT expression and/or activity is an anti-TIGIT antibody or antigen-binding fragment thereof. In some embodiments, the antagonist of TIGIT expression and/or activity is an inhibitory nucleic acid selected from an antisense polynucleotide, an interfering RNA, a catalytic RNA, and an RNA-DNA chimera. In some embodiments, the antagonist of PVR expression and/or activity is a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of TIGIT with PVR is a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of TIGIT with PVRL2 is a small molecule inhibitor, an inhibitory antibody or antigen-binding fragment thereof, an aptamer, an inhibitory nucleic acid, and an inhibitory polypeptide. In some embodiments, the agent that inhibits and/or blocks the interaction of TIGIT with PVRL3 is a small molecule inhibitor, an inhibitory antibody or antigen-binding fragment thereof, an aptamer, an inhibitory nucleic acid, and an inhibitory polypeptide. In some embodiments, the agent that inhibits and/or blocks the intracellular signaling mediated by TIGIT binding to PVR is a small molecule inhibitor, an inhibitory antibody or antigen-binding fragment thereof, an aptamer, an inhibitory nucleic acid, and an inhibitory polypeptide. In some embodiments, the agent that inhibits and/or blocks the intracellular signaling mediated by TIGIT binding to PVRL2 is a small molecule inhibitor, an inhibitory antibody or antigen-binding fragment thereof, an aptamer, an inhibitory nucleic acid, and an inhibitory polypeptide. In some embodiments, the agent that inhibits and/or blocks the intracellular signaling mediated by TIGIT binding to PVRL3 is a small molecule inhibitor, an inhibitory antibody or antigen-binding fragment thereof, an aptamer, an inhibitory nucleic acid, and an inhibitory polypeptide.

In some embodiments, the antagonist of TIGIT expression and/or activity includes a small molecule inhibitor, an inhibitory antibody or antigen-binding fragment thereof, an aptamer, an inhibitory nucleic acid, and an inhibitory polypeptide. In some embodiments, the antagonist of PVR expression and/or activity includes a small molecule inhibitor, an inhibitory antibody or antigen-binding fragment thereof, an aptamer, an inhibitory nucleic acid, and an inhibitory polypeptide. In some embodiments, the agent that inhibits the intracellular signaling mediated by TIGIT binding to PVR is selected from the group consisting of a small molecule inhibitor, an inhibitory antibody or antigen-binding fragment thereof, an aptamer, an inhibitory nucleic acid, and an inhibitory polypeptide. In some embodiments, the antagonist of TIGIT expression and/or activity is an anti-TIGIT antibody, or antigen-binding fragment thereof. In some embodiments, the anti-TIGIT antibody, or antigen-binding fragment thereof, binds to the same epitope as an antibody comprising one of the following sets of six HVR sequences: (a) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or (b) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12). In some embodiments, the antagonist of TIGIT expression and/or activity is an inhibitory nucleic acid selected from an antisense polynucleotide, an interfering RNA, a catalytic RNA, and an RNA-DNA chimera.

D. Combinations of T Cell Targets for Immunoregulatory Antibody Therapy

In addition to specific antigen recognition through the TCR, T-cell activation is regulated through a balance of positive and negative signals provided by co-stimulatory receptors. These surface proteins are typically members of either the TNF receptor or B7 superfamilies. Activating co-stimulatory receptors or their ligands include CD226, CD28, OX40, GITR, CD137, CD27, HVEM, MICA, ICOS, NKG2D, and 2B4. Inhibitory co-stimulatory receptors include CTLA-4, PD-L1, PD-1, TIM-3, BTLA, VISTA, LAG-3, B7H4, and CD96. Agonistic antibodies directed against activating co-stimulatory molecules and blocking antibodies against negative co-stimulatory molecules may enhance T-cell stimulation to promote tumor destruction.

Provided herein is a method of increasing, enhancing or stimulating an immune response or function in an individual by administering to the individual an effective amount of an agent that decreases or inhibits TIGIT expression and/or activity and an agent that decreases or inhibits one or more additional immune co-inhibitory receptors. In some embodiments, the one or more additional immune co-inhibitory receptor is selected from PD-L1, PD-1, CTLA-4, LAG3, TIM3, BTLA, VISTA, B7H4, and CD96. In some embodiments, the one or more additional immune co-inhibitory receptor is selected from PD-L1, PD-1, CTLA-4, LAG3, and TIM3.

Provided herein is also a method of increasing, enhancing or stimulating an immune response or function in an individual by administering to the individual an effective amount of an agent that decreases or inhibits TIGIT expression and/or activity and an agent that increases or activates one or more additional immune co-stimulatory receptor. In some embodiments, the one or more additional immune co-stimulatory receptor or its ligand is selected from CD226, CD28, CD27, CD137, HVEM, GITR, MICA, ICOS, NKG2D, and 2B4. In some embodiments, the one or more additional immune co-stimulatory receptor is selected from CD226, CD27, CD137, HVEM and GITR. In some embodiments, the one or more additional immune co-stimulatory receptor is CD27.

E. Agonist and Antagonist Antibodies

As described above, the agonist and antagonist agents for use in the methods of the invention may be antibodies (e.g., OX40 agonist antibodies, anti-TIGIT blocking antibodies, anti-PVR/PVRL2/PVRL3 blocking antibodies, antibodies (e.g., blocking antibodies) that specifically bind to immune co-inhibitory receptor(s), and antibodies (e.g., agonist antibodies) that specifically bind to immune co-stimulatory receptors). It is expressly contemplated that such antibodies for use in any of the embodiments enumerated above may have any of the features, singly or in combination, described in Sections 1-7 below.

1. Antibody Affinity

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ NA to 10⁻¹³ M, e.g., from 10⁻⁹ M to 10⁻¹³ M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one embodiment, carboxymethylated dextran biosensor chips (CM5, BIACORE, Inc.) are activated with N-ethyl-N′ (3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio kₒₙ/k_{off}. See, for example, Chen et al., J. Mol. Biol. 293:865-881 (1999). If the on-rate exceeds 10⁶M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In any one of the above aspects, the antibody provided herein may be a multispecific antibody, for example, a bispecific antibody. Multispecific antibodies are monoclonal antibodies that have binding specificities for at least two different sites. In certain embodiments, bispecific antibodies may bind to two different epitopes of TIGIT or OX40. In certain embodiments, one of the binding specificities is for OX40 and the other is for any other antigen (e.g., a second biological molecule, such as TIGIT). Accordingly, the bispecific antibody may have binding specificity for OX40 and TIGIT; OX40 and CD226; OX40 and PVR; OX40 and PVRL2; or OX40 and PVRL3, wherein the bispecific antibody is preferably an agonist antibody for OX40 and an antagonist antibody for its second target. In some embodiments, the bispecific antibody may have binding specificity for OX40 and PD-L1; OX40 and PD-1; OX40 and CTLA-4; OX40 and LAG3; OX40 and TIM3; OX40 and BTLA; OX40 and VISTA; OX40 and B7H4; or OX40 and CD96, wherein the bispecific antibody is preferably an agonist antibody for OX40 and an antagonist antibody for its second target. In other embodiments, the bispecific antibody may have binding specificity for OX40 and CD226; OX40 and CD28; OX40 and CD27; OX40 and CD137; OX40 and HVEM; OX40 and GITR; OX40 and MICA; OX40 and ICOS; OX40 and NKG2D; or OX40 and 2B4, wherein the bispecific antibody is preferably an agonist antibody for OX40 and for its second target.

In some embodiments, one of the binding specificities of the bispecific antibody is for TIGIT and the other is for another antigen. For example, the bispecific antibody may have binding specificity for TIGIT and CD226; TIGIT and PVR; TIGIT and PVRL2; or TIGIT and PVRL3, wherein the bispecific antibody is preferably an antagonist antibody for TIGIT and for its second target. In some embodiments, the bispecific antibody may have binding specificity for TIGIT and PD-L1; TIGIT and PD-1; TIGIT and CTLA-4; TIGIT and LAG3; TIGIT and TIM3; TIGIT and BTLA; TIGIT and VISTA; TIGIT and B7H4; or TIGIT and CD96, wherein the bispecific antibody is preferably an antagonist antibody for TIGIT and for its second target. In other embodiments, the bispecific antibody may have binding specificity for TIGIT and CD226; TIGIT and CD28; TIGIT and CD27; TIGIT and CD137; TIGIT and HVEM; TIGIT and GITR; TIGIT and MICA; TIGIT and ICOS; TIGIT and NKG2D; or TIGIT and 2B4, wherein the bispecific antibody is preferably an antagonist antibody for TIGIT and an agonist antibody for its second target. In other embodiments, the bispecific antibody may have binding specificity for TIGIT that is not antagonistic in nature (i.e., the bispecific antibody does not have act as a TIGIT antagonist).

7. Antibody Variants

In certain embodiments, amino acid sequence variants of the antibodies of the invention are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

I. Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 2 under the heading of “preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 2
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

II. Glycosylation Variants

In certain embodiments, antibodies of the invention can be altered to increase or decrease the extent to which the antibody is glycosylated. Addition or deletion of glycosylation sites to an antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Led 3 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

III. Fc Region Variants

In certain embodiments, one or more amino acid modifications may be introduced into the Fc region of an antibody of the invention, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc (RIII only, whereas monocytes express Fc (RI, Fc (RII and Fc (RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half life determinations can also be performed using methods known in the art (see, e.g., Petkova, S. B. et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (La, either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### IV. Kits

In another aspect, provided is a kit comprising an OX40 binding agonist and a package insert comprising instructions for using the OX40 binding agonist in combination with an agent that decreases or inhibits TIGIT expression and/or activity to treat or delay progression of cancer in an individual or for enhancing immune function of an individual having cancer. Any of the OX40 binding agonists and/or agents that decreases or inhibits TIGIT expression and/or activity described herein may be included in the kit.

In another aspect, provided is a kit comprising an OX40 binding agonist and an agent that decreases or inhibits TIGIT expression and/or activity, and a package insert comprising instructions for using the OX40 binding agonist and the agent that decreases or inhibits TIGIT expression and/or activity to treat or delay progression of cancer in an individual or for enhancing immune function of an individual having cancer. Any of the OX40 binding agonists and/or agents that decreases or inhibits TIGIT expression and/or activity described herein may be included in the kit.

In another aspect, provided is a kit comprising an agent that decreases or inhibits TIGIT expression and/or activity and a package insert comprising instructions for using the agent that decreases or inhibits TIGIT expression and/or activity in combination with an OX40 binding agonist to treat or delay progression of cancer in an individual or for enhancing immune function of an individual having cancer. Any of the OX40 binding agonists and/or agents that decreases or inhibits TIGIT expression and/or activity described herein may be included in the kit.

In another aspect, provided is a kit comprising an OX40 binding agonist and a package insert comprising instructions for using the OX40 binding agonist in combination with an agent that modulates the CD226 expression and/or activity to treat or delay progression of cancer in an individual. Any of the OX40 binding agonists and/or agents that modulate the CD226 expression and/or activity described herein may be included in the kit.

In another aspect, provided is a kit comprising an OX40 binding agonist and an agent that modulates the CD226 expression and/or activity, and a package insert comprising instructions for using the OX40 binding agonist and the agent that modulates the CD226 expression and/or activity to treat or delay progression of cancer in an individual. Any of the OX40 binding agonists and/or agents that modulate the CD226 expression and/or activity described herein may be included in the kit.

In another aspect, provided is a kit comprising an agent that modulates the CD226 expression and/or activity and a package insert comprising instructions for using the agent modulates the CD226 expression and/or activity in combination with an OX40 binding agonist to treat or delay progression of cancer in an individual. Any of the OX40 binding agonists and/or agents that modulate the CD226 expression and/or activity described herein may be included in the kit.

In another aspect, provided is a kit comprising an OX40 binding agonist and a package insert comprising instructions for using the OX40 binding agonist in combination with an agent that modulates the CD226 expression and/or activity to enhance immune function of an individual having cancer. Any of the OX40 binding agonists and/or agents that modulate the CD226 expression and/or activity described herein may be included in the kit.

In another aspect, provided is a kit comprising an OX40 binding agonist and an agent that modulates the CD226 expression and/or activity, and a package insert comprising instructions for using the OX40 binding agonist and the agent that modulates the CD226 expression and/or activity to enhance immune function of an individual having cancer. Any of the OX40 binding agonists and/or agents that modulate the CD226 expression and/or activity described herein may be included in the kit.

In another aspect, provided is a kit comprising an agent modulates the CD226 expression and/or activity and a package insert comprising instructions for using the agent that modulates the CD226 expression and/or activity in combination with an OX40 binding agonist to enhance immune function of an individual having cancer. Any of the OX40 binding agonists and/or agents that modulate the CD226 expression and/or activity described herein may be included in the kit.

In another aspect, provided is a kit comprising an agent that decreases or inhibits TIGIT expression and/or activity and a package insert comprising instructions for using the agent that decreases or inhibits TIGIT expression and/or activity in combination with an agent that decreases or inhibits one or more additional immune co-inhibitory receptors to treat or delay progression of cancer in an individual or to enhance immune function of an individual having cancer. Any of the agents that decrease or inhibit TIGIT expression and/or activity and/or agents that decrease or inhibit one or more additional immune co-inhibitory receptors described herein may be included in the kit.

In another aspect, provided is a kit comprising an agent that decreases or inhibits TIGIT expression and/or activity and an agent that decreases or inhibits one or more additional immune co-inhibitory receptors, and a package insert comprising instructions for using the agent that decreases or inhibits TIGIT expression and/or activity and the agent that decreases or inhibits one or more additional immune co-inhibitory receptors to treat or delay progression of cancer in an individual or to enhance immune function of an individual having cancer. Any of the agents that decrease or inhibit TIGIT expression and/or activity and/or agents that decrease or inhibit one or more additional immune co-inhibitory receptors described herein may be included in the kit.

In another aspect, provided is a kit comprising an agent that decreases or inhibits one or more additional immune co-inhibitory receptors and a package insert comprising instructions for using the agent that decreases or inhibits one or more additional immune co-inhibitory receptors in combination with an agent that decreases or inhibits TIGIT expression and/or activity to treat or delay progression of cancer in an individual or to enhance immune function of an individual having cancer. Any of the agents that decrease or inhibit TIGIT expression and/or activity and/or agents that decrease or inhibit one or more additional immune co-inhibitory receptors described herein may be included in the kit.

In another aspect, provided is a kit comprising an agent that decreases or inhibits TIGIT expression and/or activity and a package insert comprising instructions for using the agent that decreases or inhibits TIGIT expression and/or activity in combination with an agent that increases or activates one or more additional immune co-stimulatory receptors to treat or delay progression of cancer in an individual or to enhance immune function of an individual having cancer. Any of the agents that decrease or inhibit TIGIT expression and/or activity and/or agents that increase or activate one or more additional immune co-stimulatory receptors described herein may be included in the kit.

In another aspect, provided is a kit comprising an agent that decreases or inhibits TIGIT expression and/or activity and an agent that increases or activates one or more additional immune co-stimulatory receptors, and a package insert comprising instructions for using the agent that decreases or inhibits TIGIT expression and/or activity and the agent that increases or activates one or more additional immune co-stimulatory receptors to treat or delay progression of cancer in an individual or to enhance immune function of an individual having cancer. Any of the agents that decrease or inhibit TIGIT expression and/or activity and/or agents that increase or activate one or more additional immune co-stimulatory receptors described herein may be included in the kit.

In another aspect, provided is a kit comprising an agent that increases or activates one or more additional immune co-stimulatory receptors and a package insert comprising instructions for using the agent that increases or activates one or more additional immune co-stimulatory receptors in combination with an agent that decreases or inhibits TIGIT expression and/or activity to treat or delay progression of cancer in an individual or to enhance immune function of an individual having cancer. Any of the agents that decrease or inhibit TIGIT expression and/or activity and/or agents that increase or activate one or more additional immune co-stimulatory receptors described herein may be included in the kit.

In some embodiments, the kit comprises a container containing one or more of the OX40 binding agonists and agents that decreases or inhibits TIGIT expression and/or activity described herein. In some embodiments, the kit comprises a container containing one or more of the OX40 binding agonists and agents that modulates CD226 expression and/or activity described herein. In some embodiments, the kit comprises a container containing one or more of the agents that decrease or inhibit TIGIT expression and/or activity and agents that decrease or inhibit one or more additional immune co-inhibitory receptors described herein. In some embodiments, the kit comprises a container containing one or more of the agents that decrease or inhibit TIGIT expression and/or activity and agents that increase or activate one or more additional immune co-stimulatory receptors described herein. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises further cytotoxic or chemotherapeutic agent(s) or otherwise therapeutic agent(s). The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

### Example 1. Combination Treatment of Anti-OX40 Agonist Antibody and Anti-TIGIT Blocking Antibody Shows Improved Anti-Tumor Efficacy In Vivo

For the experiments described below, a blocking anti-TIGIT IgG2a monoclonal antibody (clone 10A7, reactive against both mouse and human TIGIT) was generated as previously described (Yu, X. et al. Nature Immunology. 10, 48-57, 2009) and cloned onto a murine IgG2a isotype. An agonist anti-OX40 IgG2a monoclonal antibody (clone OX-86) was also cloned onto a murine IgG2a isotype.

BALB/c mice were subcutaneously inoculated with 1×10³ CT26 colon carcinoma cells suspended in 100 μl matrigel (BD Biosciences) into the right unilateral thoracic flank. After two weeks, mice bearing tumors of approximately 150-180 mm³ were randomly recruited into four treatment groups receiving (1) 10 mg/kg of isotype control antibody, (2) 0.1 mg/kg anti-OX40 antibody (clone OX-86), (3) 10 mg/kg anti-TIGIT antibody (clone 10A7), or (4) both 0.1 mg/kg anti-OX40 antibody (clone OX-86) and 10 mg/kg anti-TIGIT antibody (clone 10A7). The anti-OX40 antibody was administered by intravenous injection once. The anti-TIGIT and control antibodies were administered by intravenous injection once followed by intraperitoneal injection 3 times per week for 3 weeks. Tumors were measured 2 times per week by caliper. Tumor volumes were calculated using the modified ellipsoid formula, ½×(length×width²). Animals whose tumors became ulcerated/necrotic or grew larger than 2000 mm³ were euthanized.

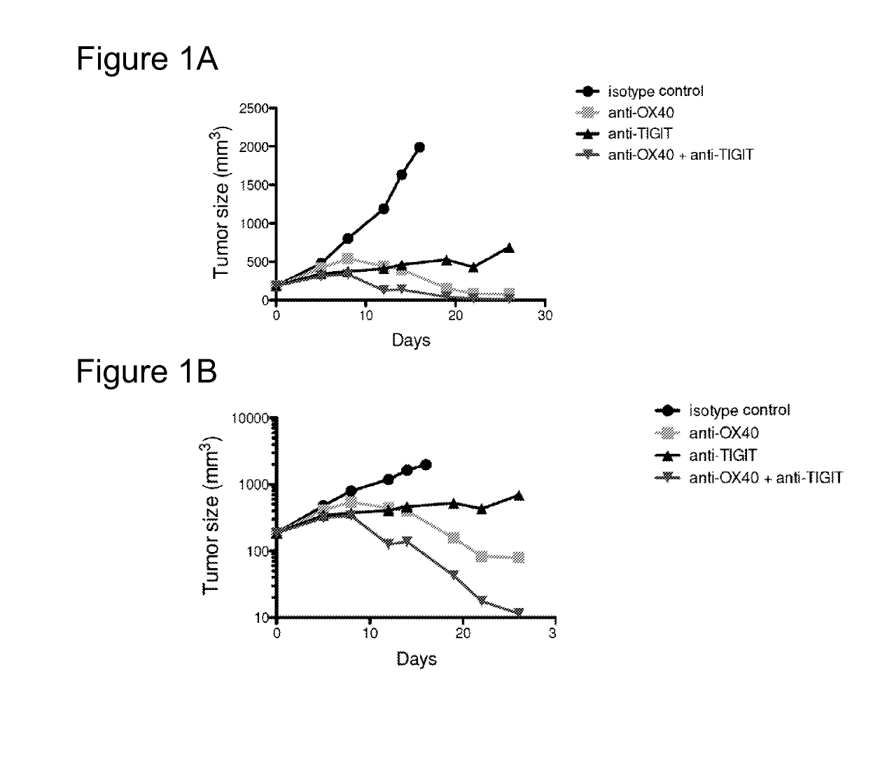

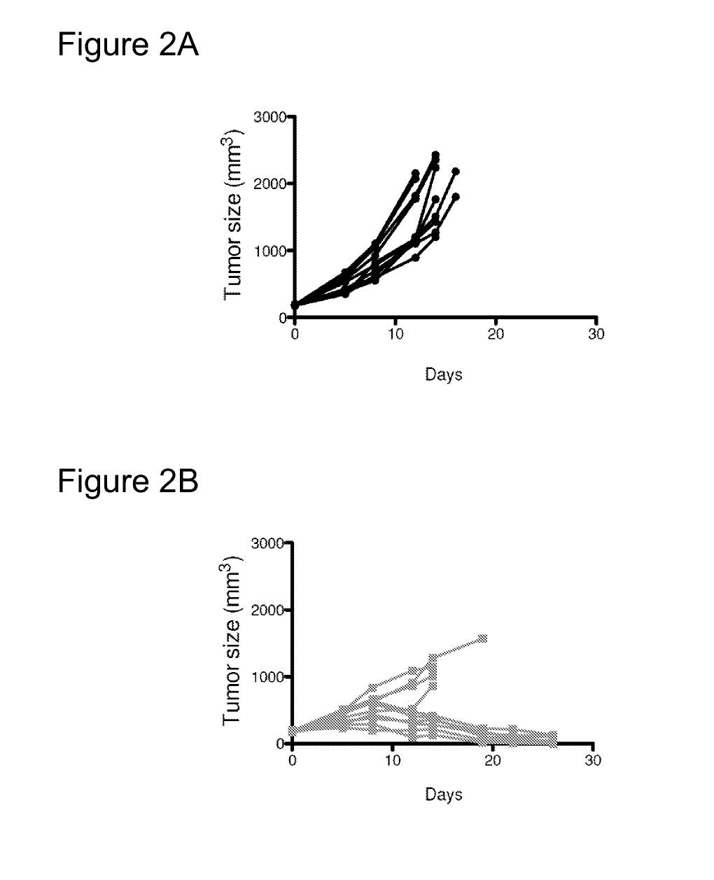

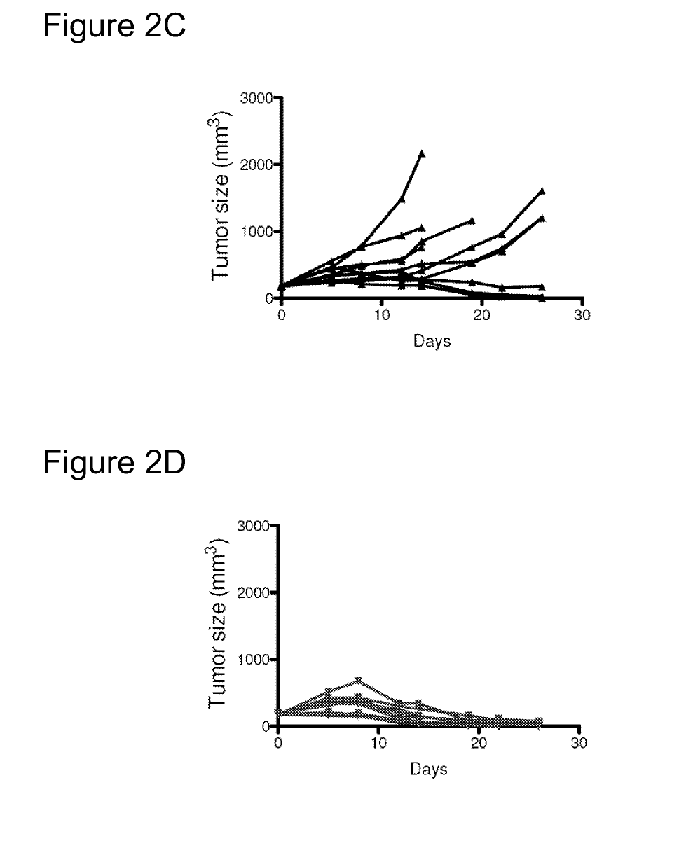

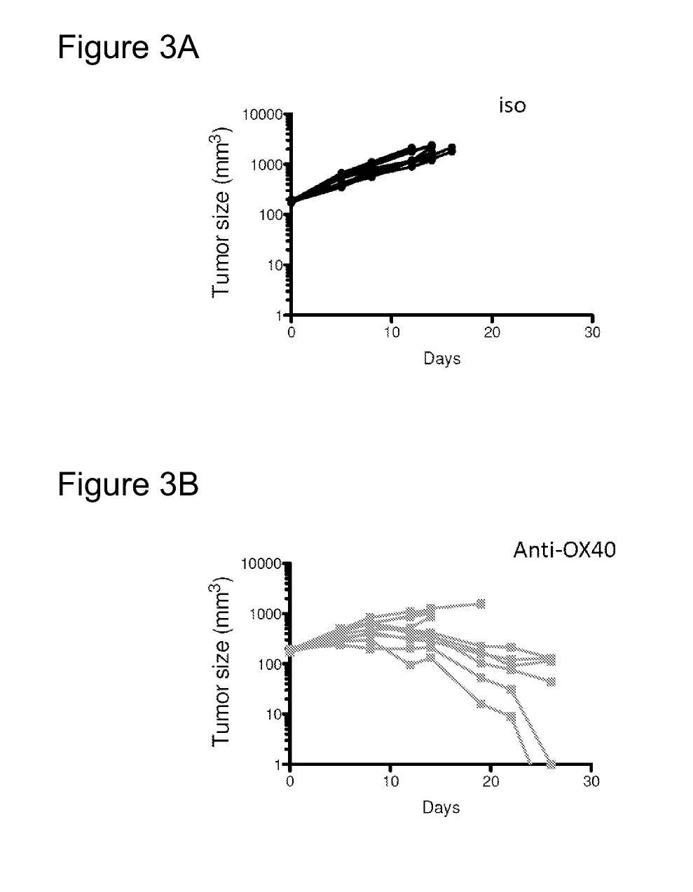

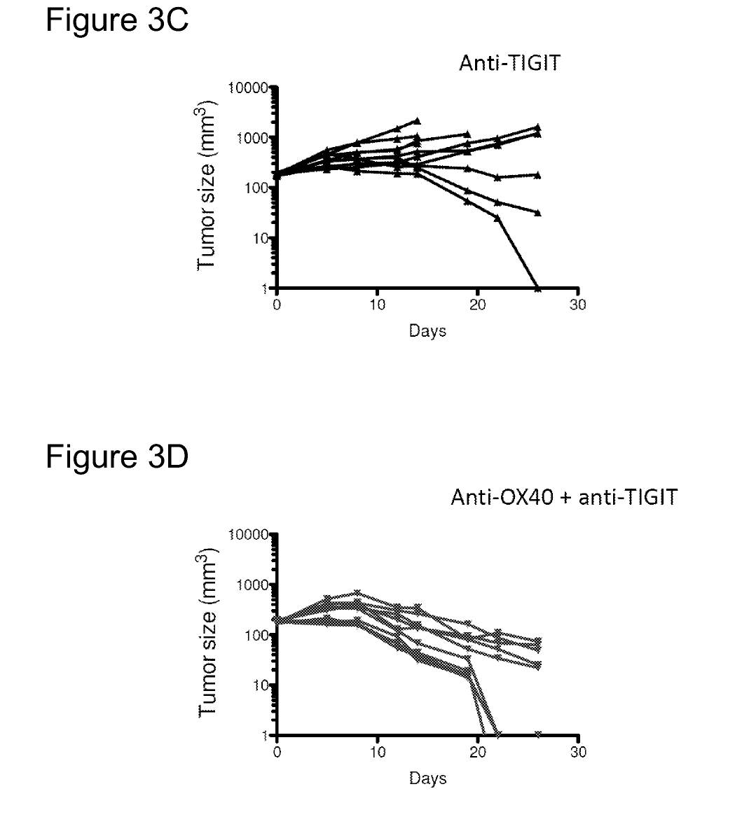

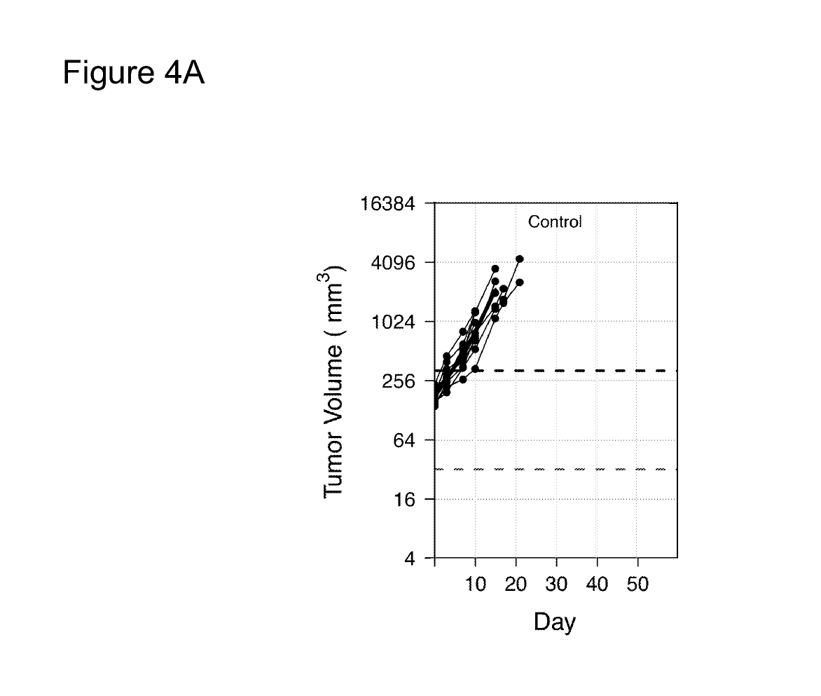

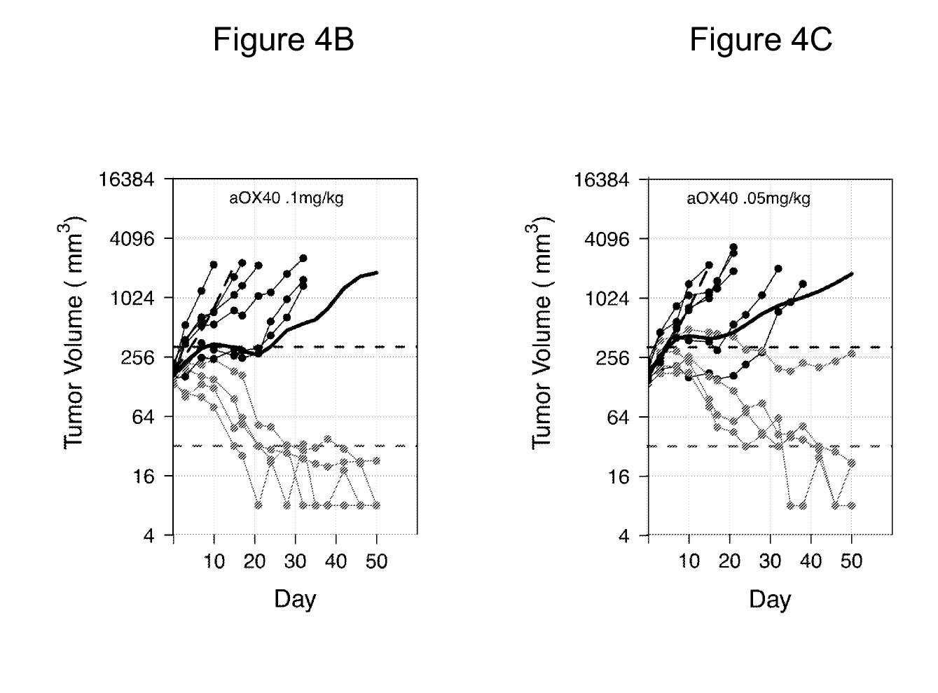

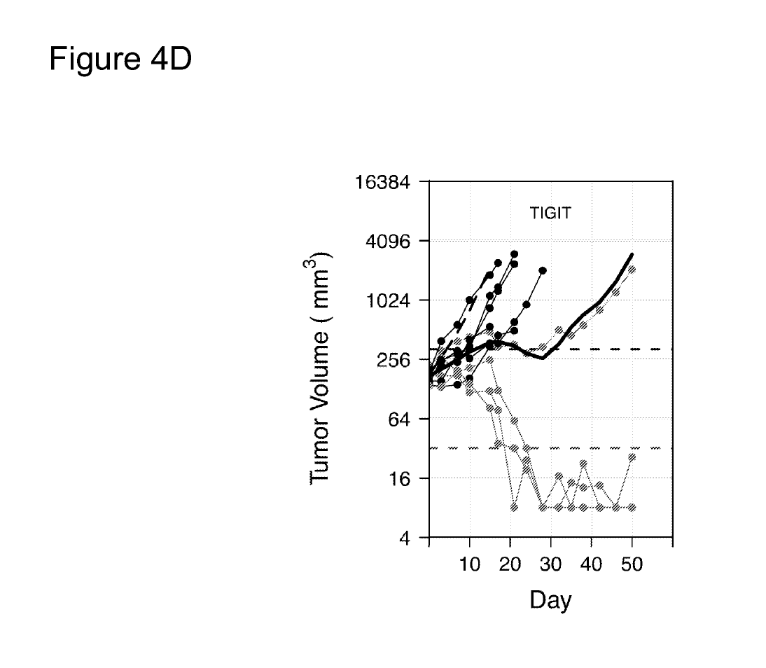

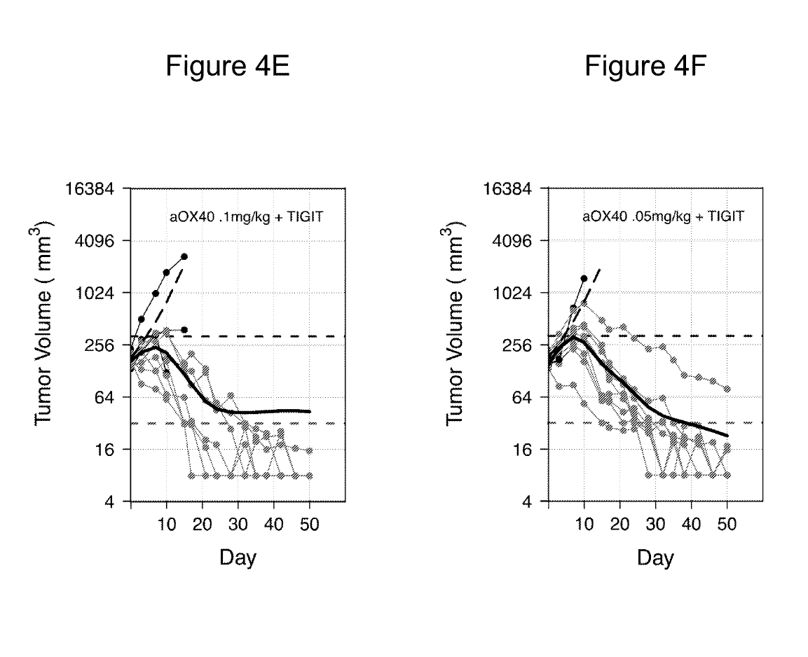

Combined treatment with both anti-OX40 agonist antibody and anti-TIGIT blocking antibody resulted in improved anti-tumor efficacy over treatment with the isotype control antibody, anti-OX40 antibody, or anti-TIGIT antibody alone (FIGS. 1-3). These results were also confirmed in a separate study (FIG. 4) using the same CT26 BALB/c mouse model in which the anti-OX40 agonist antibody (clone OX-86) was administered once by intravenous injection either at 0.1 mg/kg (high dose), as in the study above, or at 0.05 mg/kg (low dose), alone (FIGS. 4B and 4C) or in combination with the anti-TIGIT blocking antibody (clone 10A7, administered by intraperitoneal injection 3 times per week for 3 weeks; FIGS. 4E and 4F). At either low or high dose of anti-OX40 agonist antibody, the combination treatment of anti-OX40 agonist antibody and anti-TIGIT blocking antibody resulted in increased tumor regression compared to isotype control antibody, anti-OX40 antibody, or anti-TIGIT antibody alone (FIGS. 4A-4F). Collectively, these data show that the particular combination of anti-OX40 agonist antibody and anti-TIGIT blocking antibody is effective in inhibiting and tumor growth and decreasing tumor size in vivo.

### Other Embodiments

Although the foregoing invention has been described in some detail by way of illustration and example for purposes of clarity of understanding, the descriptions and examples should not be construed as limiting the scope of the invention. It is understood that various other embodiments may be practiced, given the general description provided above. The disclosures of all patent and scientific literature cited herein are expressly incorporated in their entirety by reference.

What is claimed is:

1. A method for treating or delaying progression of cancer in an individual comprising administering to the individual an effective amount of an OX40 binding agonist and an agent that decreases or inhibits TIGIT expression and/or activity.

2. A method for reducing or inhibiting cancer relapse or cancer progression in an individual comprising administering to the individual an effective amount of an OX40 binding agonist and an agent that decreases or inhibits TIGIT expression and/or activity.

3. A method for treating or delaying progression of an immune related disease in an individual comprising administering to the individual an effective amount of an OX40 binding agonist and an agent that decreases or inhibits TIGIT expression and/or activity.

4. A method for reducing or inhibiting progression of an immune related disease in an individual comprising administering to the individual an effective amount of an OX40 binding agonist and an agent that decreases or inhibits TIGIT expression and/or activity.

5. The method of claim 3 or 4, wherein the immune related disease is associated with a T cell dysfunctional disorder.

6. The method of claim 5, wherein the T cell dysfunctional disorder is characterized by decreased responsiveness to antigenic stimulation.

7. The method of claim 5, wherein the T cell dysfunctional disorder is characterized by T cell anergy or decreased ability to secrete cytokines, proliferate, or execute cytolytic activity.

8. The method of claim 5, wherein the T cell dysfunctional disorder is characterized by T cell exhaustion.

9. The method of any one of claims 3-8, wherein the T cells are CD4+ and CD8+ T cells.

10. The method of any one of claims 3-9, wherein the immune related disease is selected from the group consisting of unresolved acute infection, chronic infection, and tumor immunity.

11. A method of increasing, enhancing, or stimulating an immune response or function in an individual comprising administering to the individual an effective amount of an OX40 binding agonist and an agent that decreases or inhibits TIGIT expression and/or activity.

12. A method of treating or delaying progression of cancer in an individual comprising administering to the individual an effective amount of an OX40 binding agonist and an agent that modulates CD226 expression and/or activity.

13. A method for reducing or inhibiting cancer relapse or cancer progression in an individual comprising administering to the individual an effective amount of an OX40 binding agonist and an agent that modulates CD226 expression and/or activity.

14. A method for treating or delaying progression of an immune related disease in an individual comprising administering to the individual an effective amount of an OX40 binding agonist and an agent that modulates CD226 expression and/or activity.

15. A method for reducing or inhibiting progression of an immune related disease in an individual comprising administering to the individual an effective amount of an OX40 binding agonist and an agent that modulates CD226 expression and/or activity.

16. The method of claim 14 or 15, wherein the immune related disease is associated with a T cell dysfunctional disorder.

17. The method of claim 16, wherein the T cell dysfunctional disorder is characterized by decreased responsiveness to antigenic stimulation.

18. The method of claim 16, wherein the T cell dysfunctional disorder is characterized by T cell anergy or decreased ability to secrete cytokines, proliferate, or execute cytolytic activity.

19. The method of claim 16, wherein the T cell dysfunctional disorder is characterized by T cell exhaustion.

20. The method of any one of claims 16-19, wherein the T cell is a CD4+ T cell and/or a CD8+ T cell.

21. The method of any one of claims 14-20, wherein the immune related disease is selected from the group consisting of unresolved acute infection, chronic infection, and tumor immunity.

22. A method of increasing, enhancing, or stimulating an immune response or function in an individual comprising administering to the individual an effective amount of an OX40 binding agonist and an agent that modulates CD226 expression and/or activity.

23. The method of any one of claims 12-22, wherein the agent that modulates CD226 expression and/or activity is an agent that increases and/or stimulates CD226 expression and/or activity.

24. The method of any one of claims 12-23, wherein the agent that modulates CD226 expression and/or activity is an agent that increases and/or stimulates the interaction of CD226 with PVR.

25. The method of any one of claims 12-24, wherein the agent that modulates CD226 expression and/or activity is an agent that increases and/or stimulates the intracellular signaling mediated by CD226 binding to PVR.

26. The method of any one of claims 12-25, wherein the agent that modulates CD226 expression and/or activity is selected from the group consisting of an agent that inhibits and/or blocks the interaction of CD226 with TIGIT, an antagonist of TIGIT expression and/or activity, an antagonist of PVR expression and/or activity, an agent that inhibits and/or blocks the interaction of TIGIT with PVR, an agent that inhibits and/or blocks the interaction of TIGIT with PVRL2, an agent that inhibits and/or blocks the interaction of TIGIT with PVRL3, an agent that inhibits and/or blocks the intracellular signaling mediated by TIGIT binding to PVR, an agent that inhibits and/or blocks the intracellular signaling mediated by TIGIT binding to PVRL2, an agent that inhibits and/or blocks the intracellular signaling mediated by TIGIT binding to PVRL3, and combinations thereof.

27. The method of claim 26, wherein the agent that modulates CD226 expression and/or activity is an agent that inhibits and/or blocks the interaction of CD226 with TIGIT.

28. The method of claim 26 or 27, wherein the agent that inhibits and/or blocks the interaction of CD226 with TIGIT is a small molecule inhibitor, an inhibitory antibody or antigen-binding fragment thereof, an aptamer, an inhibitory nucleic acid, or an inhibitory polypeptide.

29. The method of claim 26 or 27, wherein the agent that inhibits and/or blocks the interaction of CD226 with TIGIT is an anti-TIGIT antibody or antigen-binding fragment thereof.

30. The method of claim 26 or 27, wherein the agent that inhibits and/or blocks the interaction of CD226 with TIGIT is an inhibitory nucleic acid selected from the group consisting of an antisense polynucleotide, an interfering RNA, a catalytic RNA, and an RNA-DNA chimera.

31. The method of claim 26, wherein the agent that modulates CD226 expression and/or activity is an antagonist of TIGIT expression and/or activity.

32. The method of claim 26 or 31, wherein the antagonist of TIGIT expression and/or activity is a small molecule inhibitor, an inhibitory antibody or antigen-binding fragment thereof, an aptamer, an inhibitory nucleic acid, and an inhibitory polypeptide.

33. The method of claim 26 or 31, wherein the antagonist of TIGIT expression and/or activity is an anti-TIGIT antibody or antigen-binding fragment thereof.

34. The method of claim 26 or 31, wherein the antagonist of TIGIT expression and/or activity is an inhibitory nucleic acid selected from the group consisting of an antisense polynucleotide, an interfering RNA, a catalytic RNA, and an RNA-DNA chimera.

35. The method of claim 26, wherein the antagonist of PVR expression and/or activity is selected from the group consisting of a small molecule inhibitor, an inhibitory antibody or antigen-binding fragment thereof, an aptamer, an inhibitory nucleic acid, and an inhibitory polypeptide.

36. The method of claim 26, wherein the agent that inhibits and/or blocks the interaction of TIGIT with PVR is selected from the group consisting of a small molecule inhibitor, an inhibitory antibody or antigen-binding fragment thereof, an aptamer, an inhibitory nucleic acid, and an inhibitory polypeptide.

37. The method of claim 26, wherein the agent that inhibits and/or blocks the interaction of TIGIT with PVRL2 is selected from the group consisting of a small molecule inhibitor, an inhibitory antibody or antigen-binding fragment thereof, an aptamer, an inhibitory nucleic acid, and an inhibitory polypeptide.

38. The method of claim 26, wherein the agent that inhibits and/or blocks the interaction of TIGIT with PVRL3 is selected from the group consisting of a small molecule inhibitor, an inhibitory antibody or antigen-binding fragment thereof, an aptamer, an inhibitory nucleic acid, and an inhibitory polypeptide.

39. The method of claim 26, wherein the agent that inhibits and/or blocks the intracellular signaling mediated by TIGIT binding to PVR is selected from the group consisting of a small molecule inhibitor, an inhibitory antibody or antigen-binding fragment thereof, an aptamer, an inhibitory nucleic acid, and an inhibitory polypeptide.

40. The method of claim 26, wherein the agent that inhibits and/or blocks the interaction of TIGIT with PVRL2 is selected from the group consisting of a small molecule inhibitor, an inhibitory antibody or antigen-binding fragment thereof, an aptamer, an inhibitory nucleic acid, and an inhibitory polypeptide.

41. The method of claim 26, wherein the agent that inhibits and/or blocks the interaction of TIGIT with PVRL3 is selected from the group consisting of a small molecule inhibitor, an inhibitory antibody or antigen-binding fragment thereof, an aptamer, an inhibitory nucleic acid, and an inhibitory polypeptide.

42. A method of increasing, enhancing, or stimulating an immune response or function in an individual comprising administering to the individual an effective amount of an OX40 binding agonist, an effective amount of an agent that decreases or inhibits TIGIT expression and/or activity, and an agent that decreases or inhibits one or more additional immune co-inhibitory receptors.

43. The method of claim 42, wherein the one or more additional immune co-inhibitory receptor is selected from the group consisting of PD-L1, PD-1, CTLA-4, LAG3, TIM3, BTLA, VISTA, B7H4, and CD96.

44. The method of claim 42, wherein the one or more additional immune co-inhibitory receptor is selected from the group consisting of PD-L1, PD-1, CTLA-4, LAG3, and TIM3.

45. A method of increasing, enhancing, or stimulating an immune response or function in an individual comprising administering to the individual an effective amount of an OX40 binding agonist, an effective amount of an agent that decreases or inhibits TIGIT expression and/or activity, and an agent that increases or activates one or more additional immune co-stimulatory receptors or their ligands.

46. The method of claim 45, wherein the one or more additional immune co-stimulatory receptors or their ligands is selected from the group consisting of CD226, CD28, CD27, CD137, HVEM, GITR, MICA, ICOS, NKG2D, and 2B4.

47. The method of claim 45, wherein the one or more additional immune co-stimulatory receptors or their ligands is selected from the group consisting of CD226, CD27, CD137, HVEM, and GITR.

48. The method of claim 45, wherein the one or more additional immune co-stimulatory receptors or their ligands is CD27.

49. The method of any one of the preceding claims, further comprising administering at least one chemotherapeutic agent.

50. The method of any one of the preceding claims, wherein the individual has cancer.

51. The method of any one of the preceding claims, wherein CD4 and/or CD8 T cells in the individual have increased or enhanced priming, activation, proliferation, cytokine release, and/or cytolytic activity relative to prior to the administration of the combination.

52. The method of any one of the preceding claims, wherein the number of CD4 and/or CD8 T cells is elevated relative to prior to administration of the combination.

53. The method of any one of the preceding claims, wherein the number of activated CD4 and/or CD8 T cells is elevated relative to prior to administration of the combination.

54. The method of any one of the preceding claims, wherein activated CD4 and/or CD8 T cells are characterized by IFN-γ⁺ producing CD4 and/or CD8 T cells and/or enhanced cytolytic activity relative to prior to the administration of the combination.

55. The method of any one of claims 51-54, wherein the CD4 and/or CD8 T cells exhibit increased release of cytokines selected from the group consisting of IFN-γ, TNF-α, and interleukins.

56. The method of any one of claims 51-55, wherein the CD4 and/or CD8 T cells are effector memory T cells.

57. The method of claim 56, wherein the CD4 and/or CD8 effector memory T cells are characterized by γ-IFN⁺ producing CD4 and/or CD8 T cells and/or enhanced cytolytic activity.

58. The method of claim 56, wherein the CD4 and/or CD8 effector memory T cells are characterized by having the expression of CD44^{high} CD62L^{low}.

59. The method of any one of claims 1, 2, 12, 13, 23-24, and 49-58, wherein the cancer has elevated levels of T cell infiltration.

60. The method of any one of claims 1-11 and 42-59, wherein the agent that decreases or inhibits TIGIT expression and/or activity is selected from the group consisting of an antagonist of TIGIT expression and/or activity, an antagonist of PVR expression and/or activity, an agent that inhibits and/or blocks the interaction of TIGIT with PVR, an agent that inhibits and/or blocks the interaction of TIGIT with PVRL2, an agent that inhibits and/or blocks the interaction of TIGIT with PVRL3, an agent that inhibits and/or blocks the intracellular signaling mediated by TIGIT binding to PVR, an agent that inhibits and/or blocks the intracellular signaling mediated by TIGIT binding to PVRL2, an agent that inhibits and/or blocks the intracellular signaling mediated by TIGIT binding to PVRL3, and combinations thereof.

61. The method of claim 60, wherein the antagonist of TIGIT expression and/or activity is selected from the group consisting of a small molecule inhibitor, an inhibitory antibody or antigen-binding fragment thereof, an aptamer, an inhibitory nucleic acid, and an inhibitory polypeptide.

62. The method of claim 60, wherein the antagonist of PVR expression and/or activity is selected from the group consisting of a small molecule inhibitor, an inhibitory antibody or antigen-binding fragment thereof, an aptamer, an inhibitory nucleic acid, and an inhibitory polypeptide.

63. The method of claim 60, wherein the agent that inhibits and/or blocks the interaction of TIGIT with PVR is selected from the group consisting of a small molecule inhibitor, an inhibitory antibody or antigen-binding fragment thereof, an aptamer, an inhibitory nucleic acid, and an inhibitory polypeptide.

64. The method of claim 60, wherein the agent that inhibits and/or blocks the interaction of TIGIT with PVRL2 is selected from the group consisting of a small molecule inhibitor, an inhibitory antibody or antigen-binding fragment thereof, an aptamer, an inhibitory nucleic acid, and an inhibitory polypeptide.

65. The method of claim 60, wherein the agent that inhibits and/or blocks the interaction of TIGIT with PVRL3 is selected from the group consisting of a small molecule inhibitor, an inhibitory antibody or antigen-binding fragment thereof, an aptamer, an inhibitory nucleic acid, and an inhibitory polypeptide.

66. The method of claim 60, wherein the agent that inhibits and/or blocks the intracellular signaling mediated by TIGIT binding to PVR is selected from the group consisting of a small molecule inhibitor, an inhibitory antibody or antigen-binding fragment thereof, an aptamer, an inhibitory nucleic acid, and an inhibitory polypeptide.

67. The method of claim 60, wherein the agent that inhibits and/or blocks the intracellular signaling mediated by TIGIT binding to PVRL2 is selected from the group consisting of a small molecule inhibitor, an inhibitory antibody or antigen-binding fragment thereof, an aptamer, an inhibitory nucleic acid, and an inhibitory polypeptide.

68. The method of claim 60, wherein the agent that inhibits and/or blocks the intracellular signaling mediated by TIGIT binding to PVRL3 is selected from the group consisting of a small molecule inhibitor, an inhibitory antibody or antigen-binding fragment thereof, an aptamer, an inhibitory nucleic acid, and an inhibitory polypeptide.

69. The method of claim 60 or 61, wherein the antagonist of TIGIT expression and/or activity is an inhibitory nucleic acid selected from the group consisting of an antisense polynucleotide, an interfering RNA, a catalytic RNA, and an RNA-DNA chimera.

70. The method of claim 60 or 61, wherein the antagonist of TIGIT expression and/or activity is an anti-TIGIT antibody, or antigen-binding fragment thereof.

71. The method of claim 29 or 70, wherein the anti-TIGIT antibody, or antigen-binding fragment thereof, comprises at least one HVR comprising an amino acid sequence selected from the amino acid sequences:
(a) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or
(b) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12).

72. The method of claim 71, wherein the anti-TIGIT antibody, or antigen-binding fragment thereof, comprises one of the following sets of six HVR sequences:
(a) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or
(b) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12).

73. The method of any one of claims 29 and 70-72, wherein the anti-TIGIT antibody, or antigen-binding fragment thereof, comprises a light chain comprising the amino acid sequence set forth in DIVMTQSPSSLAVSPGEKVTMTCKSSQSLYYSGVKENLLAWYQQKPGQSPKLLIYYASIRFTGVPDRFTG SGSGTDYTLTITSVQAEDMGQYFCQQGINNPLTFGDGTKLEIKR (SEQ ID NO:13) or DVVLTQTPLSLSVSFGDQVSISCRSSQSLVNSYGNTFLSWYLHKPGQSPQLLIFGISNRFSGVPDRFSGS GSGTDFTLKISTIKPEDLGMYYCLQGTHQPPTFGPGTKLEVK (SEQ ID NO:14).

74. The method of any one of claims 29 and 70-73, wherein the anti-TIGIT antibody, or antigen-binding fragment thereof, comprises a heavy chain comprising the amino acid sequence set forth in EVQLVESGGGLTQPGKSLKLSCEASGFTFSSFTMHWVRQSPGKGLEWVAFIRSGSGIVFYADAVRGRFT ISRDNAKNLLFLQMNDLKSEDTAMYYCARRPLGHNTFDSWGQGTLVTVSS (SEQ ID NO:15) or EVQLQQSGPELVKPGTSMKISCKASGYSFTGHLMNWVKQSHGKNLEWIGLIIPYNGGTSYNQKFKGKAT LTVDKSSSTAYMELLSLTSDDSAVYFCSRGLRGFYAMDYWGQGTSVTVSS (SEQ ID NO:16).

75. The method of any one of claims 29 and 70-74, wherein the anti-TIGIT antibody, or antigen-binding fragment thereof, comprises a light chain comprising the amino acid sequence set forth in DIVMTQSPSSLAVSPGEKVTMTCKSSQSLYYSGVKENLLAWYQQKPGQSPKLLIYYASIRFTGVPDRFTG SGSGTDYTLTITSVQAEDMGQYFCQQGINNPLTFGDGTKLEIKR (SEQ ID NO:13) or DVVLTQTPLSLSVSFGDQVSISCRSSQSLVNSYGNTFLSWYLHKPGQSPQLLIFGISNRFSGVPDRFSGS GSGTDFTLKISTIKPEDLGMYYCLQGTHQPPTFGPGTKLEVK (SEQ ID NO:14), and
a heavy chain comprising the amino acid sequence set forth in EVQLVESGGGLTQPGKSLKLSCEASGFTFSSFTMHWVRQSPGKGLEWVAFIRSGSGIVFYADAVRGRFT ISRDNAKNLLFLQMNDLKSEDTAMYYCARRPLGHNTFDSWGQGTLVTVSS (SEQ ID NO:15) or EVQLQQSGPELVKPGTSMKISCKASGYSFTGHLMNWVKQSHGKNLEWIGLIIPYNGGTSYNQKFKGKAT LTVDKSSSTAYMELLSLTSDDSAVYFCSRGLRGFYAMDYWGQGTSVTVSS (SEQ ID NO: 16).

76. The method of any one of claims 29 and 70-75, wherein the anti-TIGIT antibody, or antigen-binding fragment thereof, wherein the antibody is selected from the group consisting of a humanized antibody, a chimeric antibody, a bispecific antibody, a heteroconjugate antibody, and an immunotoxin.

77. The method of any one of claims 29 and 70-76, wherein the anti-TIGIT antibody, or antigen-binding fragment thereof, comprises at least one HVR that is at least 90% identical to an HVR set forth in any one of KSSQSLYYSGVKENLLA (SEQ ID NO: 1); ASIRFT (SEQ ID NO: 2); QQGINNPLT (SEQ ID NO: 3); GFTFSSFTMH (SEQ ID NO: 4); FIRSGSGIVFYADAVRG (SEQ ID NO: 5); RPLGHNTFDS (SEQ ID NO: 6); RSSQSLVNSYGNTFLS (SEQ ID NO: 7); GISNRFS (SEQ ID NO: 8); LQGTHQPPT (SEQ ID NO: 9); GYSFTGHLMN (SEQ ID NO: 10); LIIPYNGGTSYNQKFKG (SEQ ID NO: 11); and GLRGFYAMDY (SEQ ID NO: 12).

78. The method of any one of claims 29, 70-72, and 77, wherein the anti-TIGIT antibody, or antigen-binding fragment thereof, comprises a light chain comprising amino acid sequences at least 90% identical to the amino acid sequences set forth in DIVMTQSPSSLAVSPGEKVTMTCKSSQSLYYSGVKENLLAWYQQKPGQSPKLLIYYASIRFTGVPDRFTG SGSGTDYTLTITSVQAEDMGQYFCQQGINNPLTFGDGTKLEIKR (SEQ ID NO:13) or DVVLTQTPLSLSVSFGDQVSISCRSSQSLVNSYGNTFLSWYLHKPGQSPQLLIFGISNRFSGVPDRFSGS GSGTDFTLKISTIKPEDLGMYYCLQGTHQPPTFGPGTKLEVK (SEQ ID NO:14); and/or
comprises a heavy chain comprising amino acid sequences at least 90% identical to the amino acid sequences set forth in EVQLVESGGGLTQPGKSLKLSCEASGFTFSSFTMHWVRQSPGKGLEWVAFIRSGSGIVFYADAVRGRFT ISRDNAKNLLFLQMNDLKSEDTAMYYCARRPLGHNTFDSWGQGTLVTVSS (SEQ ID NO:15) or EVQLQQSGPELVKPGTSMKISCKASGYSFTGHLMNWVKQSHGKNLEWIGLIIPYNGGTSYNQKFKGKAT LTVDKSSSTAYMELLSLTSDDSAVYFCSRGLRGFYAMDYWGQGTSVTVSS (SEQ ID NO:16).

79. The method of any one of claims 29 and 70-77, wherein the anti-TIGIT antibody, or antigen-binding fragment thereof, binds to the same epitope as an antibody comprising one of the following sets of six HVR sequences:
(a) KSSQSLYYSGVKENLLA (SEQ ID NO:1), ASIRFT (SEQ ID NO:2), QQGINNPLT (SEQ ID NO:3), GFTFSSFTMH (SEQ ID NO:4), FIRSGSGIVFYADAVRG (SEQ ID NO:5), and RPLGHNTFDS (SEQ ID NO:6); or
(b) RSSQSLVNSYGNTFLS (SEQ ID NO:7), GISNRFS (SEQ ID NO:8), LQGTHQPPT (SEQ ID NO:9), GYSFTGHLMN (SEQ ID NO:10), LIIPYNGGTSYNQKFKG (SEQ ID NO:11), and GLRGFYAMDY (SEQ ID NO:12).

80. The method of any one of the preceding claims, wherein the OX40 binding agonist is selected from the group consisting of an OX40 agonist antibody, an OX40L agonist fragment, an OX40 oligomeric receptor, and an OX40 immunoadhesin.

81. The method of claim 80, wherein the OX40 agonist antibody depletes cells that express human OX40.

82. The method of claim 81, wherein the cells that express human OX40 are CD4+ effector T cells.

83. The method of claim 81, wherein the cells that express human OX40 are regulatory T (Treg) cells.

84. The method of any one of the preceding claims, wherein the depleting is by ADCC and/or phagocytosis.

85. The method of claim 84, wherein the depleting is by ADCC.

86. The method of any one of the preceding claims, wherein the OX40 agonist antibody binds human OX40 with an affinity of less than or equal to about 0.45 nM.

87. The method of claim 86, wherein the OX40 agonist antibody binds human OX40 with an affinity of less than or equal to about 0.4 nM.

88. The method of claim 86 or 87, wherein the binding affinity of the OX40 agonist antibody is determined using radioimmunoassay.

89. The method of any one of the preceding claims, wherein the OX40 agonist antibody binds human OX40 and cynomolgus OX40.

90. The method of claim 89, wherein binding is determined using a FACS assay.

91. The method of claim 89 or 90, wherein binding to human OX40 has an EC50 of less than or equal to 0.3 μg/ml.

92. The method of claim 89 or 90, wherein binding to human OX40 has an EC50 of less than or equal to 0.2 μg/ml.

93. The method of any one of claims 89-92, wherein binding to cynomolgus OX40 has an EC50 of less than or equal to 1.5 μg/ml.

94. The method of claim 93, wherein binding to cynomolgus OX40 has an EC50 of less than or equal to 1.4 μg/ml.

95. The method of any one of the preceding claims, wherein the OX40 agonist antibody increases CD4+ effector T cell proliferation and/or increases cytokine production by the CD4+ effector T cell as compared to proliferation and/or cytokine production prior to treatment with the OX40 agonist antibody.

96. The method of claim 95, wherein the cytokine is IFN-γ.

97. The method of any one of the preceding claims, wherein the OX40 agonist antibody increases memory T cell proliferation and/or increasing cytokine production by the memory cell.

98. The method of claim 97, wherein the cytokine is IFN-γ.

99. The method of any one of the preceding claims, wherein the OX40 agonist antibody inhibits Treg function.

100. The method of claim 99, wherein the OX40 agonist antibody inhibits Treg suppression of effector T cell function.

101. The method of claim 100, wherein effector T cell function is effector T cell proliferation and/or cytokine production.

102. The method of claim 100 or 101, wherein the effector T cell is a CD4+ effector T cell.

103. The method of any one of the preceding claims, wherein the OX40 agonist antibody increases OX40 signal transduction in a target cell that expresses OX40.

104. The method of claim 103, wherein OX40 signal transduction is detected by monitoring NFkB downstream signaling.

105. The method of any one of the preceding claims, wherein the OX40 agonist antibody is stable after treatment at 40° C. for two weeks.

106. The method of any one of the preceding claims, wherein the OX40 agonist antibody comprising a variant IgG1 Fc polypeptide comprising a mutation that eliminates binding to human effector cells has diminished activity relative to the OX40 agonist antibody comprising a native sequence IgG1 Fc portion.

107. The method of claim 106, wherein the OX40 agonist antibody comprises a variant Fc portion comprising a DANA mutation.

108. The method of any one of the preceding claims, wherein antibody cross-linking is required for anti-human OX40 agonist antibody function.

109. The method of any one of the preceding claims, wherein the OX40 agonist antibody comprises (a) a VH domain comprising (i) HVR-H1 comprising the amino acid sequence of SEQ ID NO: 22, 28, or 29, (ii) HVR-H2 comprising the amino acid sequence of SEQ ID NO: 23, 30, 31, 32, 33 or 34, and (iii) HVR-H3 comprising an amino acid sequence selected from SEQ ID NO: 24, 35, or 39; and (iv) HVR-L1 comprising the amino acid sequence of SEQ ID NO: 25, (v) HVR-L2 comprising the amino acid sequence of SEQ ID NO: 26, and (vi) HVR-L3 comprising the amino acid sequence of SEQ ID NO: 27, 42, 43, 44, 45, 46, 47, or 48.

110. The method of claim 109, wherein the OX40 agonist antibody comprises (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 25; (e) HVR-L2 comprising the amino acid sequence of SEQ ID NO: 26; and (f) HVR-L3 comprising an amino acid sequence selected from SEQ ID NO: 27.

111. The method of claim 109, wherein the OX40 agonist antibody comprises (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 25; (e) HVR-L2 comprising the amino acid sequence of SEQ ID NO: 26; and (f) HVR-L3 comprising an amino acid sequence selected from SEQ ID NO: 46.

112. The method of claim 109, wherein the OX40 agonist antibody comprises (a) HVR-H1 comprising the amino acid sequence of SEQ ID NO: 22; (b) HVR-H2 comprising the amino acid sequence of SEQ ID NO: 23; (c) HVR-H3 comprising the amino acid sequence of SEQ ID NO: 24; (d) HVR-L1 comprising the amino acid sequence of SEQ ID NO: 25; (e) HVR-L2 comprising the amino acid sequence of SEQ ID NO: 26; and (f) HVR-L3 comprising an amino acid sequence selected from SEQ ID NO: 47.

113. The method of any one of the preceding claims, wherein the OX40 agonist antibody comprises a VH sequence having at least 90%, 91%, 92%, 93%, 94%, 95%, 96%, 97%, 98%, 99%, or 100% sequence identity to the amino acid sequence of SEQ ID NO: 76, 78, 80, 82, 84, 86, 88, 90, 92, 94, 96, 98, 100, 102, 104, 106, 108, 110, 112, 114, 116, 118, 120, 128, 134, or 136.

114. The method of any one of the preceding claims, wherein the OX40 agonist antibody comprises a VL having at least 90%, 91%, 92%, 93%, 94%, 95%, 96%, 97%, 98%, 99%, or 100% sequence identity to the amino acid sequence of SEQ ID NO: 77, 79, 81, 83, 85, 87, 89, 91, 93, 95, 97, 99, 101, 103, 105, 107, 109, 111, 113, 115, 117, 119, 121, 129, 135, or 137.

115. The method of any one of the preceding claims, wherein the OX40 agonist antibody comprises a VH sequence having at least 90%, 91%, 92%, 93%, 94%, 95%, 96%, 97%, 98%, 99%, or 100% sequence identity to the amino acid sequence of SEQ ID NO: 76.

116. The method of claim 115, wherein the OX40 agonist antibody retains the ability to bind to human OX40.

117. The method of claim 115 or 116, wherein a total of 1 to 10 amino acids have been substituted, inserted, and/or deleted in SEQ ID NO: 76.

118. The method of any one of claims 115-117, wherein the OX40 agonist antibody comprises a VH comprising one, two, or three HVRs selected from: (a) HVR-H1 comprising the amino acid sequence of SEQ ID NO: 22, (b) HVR-H2 comprising the amino acid sequence of SEQ ID NO: 23, and (c) HVR-H3 comprising the amino acid sequence of SEQ ID NO: 24.

119. The method of any one of the preceding claims, wherein the OX40 agonist antibody comprises a VL having at least 90%, 91%, 92%, 93%, 94%, 95%, 96%, 97%, 98%, 99%, or 100% sequence identity to the amino acid sequence of SEQ ID NO: 77.

120. The method of claim 119, wherein the OX40 agonist antibody retains the ability to bind to human OX40.

121. The method of claim 119 or 120, wherein a total of 1 to 10 amino acids have been substituted, inserted, and/or deleted in SEQ ID NO: 77.

122. The method of any one of claims 119-121, wherein the OX40 agonist antibody comprises a VL comprising one, two, or three HVRs selected from (a) HVR-L1 comprising the amino acid sequence of SEQ ID NO: 25; (b) HVR-L2 comprising the amino acid sequence of SEQ ID NO: 26; and (c) HVR-L3 comprising the amino acid sequence of SEQ ID NO: 27.

123. The method of any one of the preceding claims, wherein the OX40 agonist antibody comprises a VH sequence of SEQ ID NO: 76.

124. The method of any one of the preceding claims, wherein the OX40 agonist antibody comprises a VL sequence of SEQ ID NO: 77.

125. The method of any one of the preceding claims, wherein the OX40 agonist antibody comprises a VH sequence of SEQ ID NO: 76 and a VL sequence of SEQ ID NO: 77.

126. The method of any one of claims 1-122, wherein the OX40 agonist antibody comprises a VH sequence of SEQ ID NO: 114.

127. The method of any one of claims 1-122, wherein the OX40 agonist antibody comprises a VL sequence of SEQ ID NO: 115.

128. The method of any one of claims 1-122, 126, and 127, wherein the OX40 agonist antibody comprises a VH sequence of SEQ ID NO: 114 and a VL sequence of SEQ ID NO: 115.

129. The method of any one of claims 1-122, wherein the OX40 agonist antibody comprises a VH sequence of SEQ ID NO: 116.

130. The method of any one of claims 1-122, wherein the OX40 agonist antibody comprises a VL sequence of SEQ ID NO: 117.

131. The method of any one of claims 1-122, 129, and 130, wherein the OX40 agonist antibody comprises a VH sequence of SEQ ID NO: 116 and a VL sequence of SEQ ID NO: 117.

132. The method of any one of claims 80-108, wherein the OX40 agonist antibody comprises (a) a heavy chain comprising an amino acid sequence having at least 90% sequence identity to the amino acid sequence of SEQ ID NO: 200; (b) a light chain comprising an amino acid sequence having at least 90% sequence identity to the amino acid sequence of SEQ ID NO: 201; or (c) both a heavy chain as in (a) and a light chain as in (b).

133. The method of any one of claims 80-108, wherein the OX40 agonist antibody comprises (a) a heavy chain comprising an amino acid sequence having at least 90% sequence identity to the amino acid sequence of SEQ ID NO: 203; (b) a light chain comprising an amino acid sequence having at least 90% sequence identity to the amino acid sequence of SEQ ID NO: 204; or (c) both a heavy chain as in (a) and a light chain as in (b).

134. The method of any one of claims 80-108, wherein the OX40 agonist antibody comprises (a) a VH comprising an amino acid sequence having at least 90% sequence identity to the amino acid sequence of SEQ ID NO: 205; (b) a VL comprising an amino acid sequence having at least 90% sequence identity to the amino acid sequence of SEQ ID NO: 206; or (c) both a VH as in (a) and a VL as in (b).

135. The method of any one of claims 80-108, wherein the OX40 agonist antibody comprises (a) a VH comprising an amino acid sequence having at least 90% sequence identity to the amino acid sequence of SEQ ID NO: 207; (b) a VL comprising an amino acid sequence having at least 90% sequence identity to the amino acid sequence of SEQ ID NO: 208; or (c) both a VH as in (a) and a VL as in (b).

136. The method of any one of claims 80-108, wherein the OX40 agonist antibody comprises (a) a VH comprising an amino acid sequence having at least 90% sequence identity to the amino acid sequence of SEQ ID NO: 209; (b) a VL comprising an amino acid sequence having at least 90% sequence identity to the amino acid sequence of SEQ ID NO: 210; or (c) both a VH as in (a) and a VL as in (b).

137. The method of any one of claims 80-108, wherein the OX40 agonist antibody comprises (a) a VH comprising an amino acid sequence having at least 90% sequence identity to the amino acid sequence of SEQ ID NO: 211; (b) a VL comprising an amino acid sequence having at least 90% sequence identity to the amino acid sequence of SEQ ID NO: 212; or (c) both a VH as in (a) and a VL as in (b).

138. The method of any one of claims 80-108, wherein the OX40 agonist antibody comprises (a) a heavy chain comprising an amino acid sequence having at least 90% sequence identity to the amino acid sequence of SEQ ID NO: 213; (b) a light chain comprising an amino acid sequence having at least 90% sequence identity to the amino acid sequence of SEQ ID NO: 214; or (c) both a heavy chain as in (a) and a light chain as in (b).

139. The method of any one of claims 80-108, wherein the OX40 agonist antibody comprises (a) a VH comprising an amino acid sequence having at least 90% sequence identity to the amino acid sequence of SEQ ID NO: 215; (b) a VL comprising an amino acid sequence having at least 90% sequence identity to the amino acid sequence of SEQ ID NO: 216; or (c) both a VH as in (a) and a VL as in (b).

140. The method of any one of claims 80-108, wherein the OX40 agonist antibody comprises (a) a VH comprising an amino acid sequence having at least 90% sequence identity to the amino acid sequence of SEQ ID NO: 217; (b) a VL comprising an amino acid sequence having at least 90% sequence identity to the amino acid sequence of SEQ ID NO: 218; or (c) both a VH as in (a) and a VL as in (b).

141. The method of any one of claims 80-108, wherein the OX40 agonist antibody comprises (a) a VH comprising an amino acid sequence having at least 90% sequence identity to the amino acid sequence of SEQ ID NO: 219; (b) a VL comprising an amino acid sequence having at least 90% sequence identity to the amino acid sequence of SEQ ID NO: 220; or (c) both a VH as in (a) and a VL as in (b).

142. The method of any one of claims 80-108, wherein the OX40 agonist antibody comprises (a) a VH comprising an amino acid sequence having at least 90% sequence identity to the amino acid sequence of SEQ ID NO: 219; (b) a VL comprising an amino acid sequence having at least 90% sequence identity to the amino acid sequence of SEQ ID NO: 221; or (c) both a VH as in (a) and a VL as in (b).

143. The method of any one of claims 80-108, wherein the OX40 agonist antibody comprises (a) a VH comprising an amino acid sequence having at least 90% sequence identity to the amino acid sequence of SEQ ID NO: 222; (b) a VL comprising an amino acid sequence having at least 90% sequence identity to the amino acid sequence of SEQ ID NO: 220; or (c) both a VH as in (a) and a VL as in (b).

144. The method of any one of claims 80-108, wherein the OX40 agonist antibody comprises (a) a VH comprising an amino acid sequence having at least 90% sequence identity to the amino acid sequence of SEQ ID NO: 222; (b) a VL comprising an amino acid sequence having at least 90% sequence identity to the amino acid sequence of SEQ ID NO: 221; or (c) both a VH as in (a) and a VL as in (b).

145. The method of any one of claims 80-108, wherein the OX40 agonist antibody comprises (a) a VH comprising an amino acid sequence having at least 90% sequence identity to the amino acid sequence of SEQ ID NO: 223; (b) a VL comprising an amino acid sequence having at least 90% sequence identity to the amino acid sequence of SEQ ID NO: 220; or (c) both a VH as in (a) and a VL as in (b).

146. The method of any one of claims 80-108, wherein the OX40 agonist antibody comprises (a) a VH comprising an amino acid sequence having at least 90% sequence identity to the amino acid sequence of SEQ ID NO: 223; (b) a VL comprising an amino acid sequence having at least 90% sequence identity to the amino acid sequence of SEQ ID NO: 221; or (c) both a VH as in (a) and a VL as in (b).

147. The method of any one of claims 80-108, wherein the OX40 agonist antibody comprises (a) a VH comprising an amino acid sequence having at least 90% sequence identity to the amino acid sequence of SEQ ID NO: 224; (b) a VL comprising an amino acid sequence having at least 90% sequence identity to the amino acid sequence of SEQ ID NO: 225; or (c) both a VH as in (a) and a VL as in (b).

148. The method of any one of claims 80-108, wherein the OX40 agonist antibody comprises (a) a VH comprising an amino acid sequence having at least 90% sequence identity to the amino acid sequence of SEQ ID NO: 224; (b) a VL comprising an amino acid sequence having at least 90% sequence identity to the amino acid sequence of SEQ ID NO: 226; or (c) both a VH as in (a) and a VL as in (b).

149. The method of any one of claims 80-108, wherein the OX40 agonist antibody comprises (a) a VH comprising an amino acid sequence having at least 90% sequence identity to the amino acid sequence of SEQ ID NO: 227; (b) a VL comprising an amino acid sequence having at least 90% sequence identity to the amino acid sequence of SEQ ID NO: 225; or (c) both a VH as in (a) and a VL as in (b).

150. The method of any one of claims 80-108, wherein the OX40 agonist antibody comprises (a) a VH comprising an amino acid sequence having at least 90% sequence identity to the amino acid sequence of SEQ ID NO: 227; (b) a VL comprising an amino acid sequence having at least 90% sequence identity to the amino acid sequence of SEQ ID NO: 226; or (c) both a VH as in (a) and a VL as in (b).

151. The method of any one of claims 80-108, wherein the OX40 agonist antibody comprises (a) a VH comprising an amino acid sequence having at least 90% sequence identity to the amino acid sequence of SEQ ID NO: 228; (b) a VL comprising an amino acid sequence having at least 90% sequence identity to the amino acid sequence of SEQ ID NO: 225; or (c) both a VH as in (a) and a VL as in (b).

152. The method of any one of claims 80-108, wherein the OX40 agonist antibody comprises (a) a VH comprising an amino acid sequence having at least 90% sequence identity to the amino acid sequence of SEQ ID NO: 228; (b) a VL comprising an amino acid sequence having at least 90% sequence identity to the amino acid sequence of SEQ ID NO: 226; or (c) both a VH as in (a) and a VL as in (b).

153. The method of any one of claims 80-108, wherein the OX40 agonist antibody is antibody L106, antibody ACT35, MEDI6469, or MEDI0562.

154. The method of any one of claims 80-153, wherein the OX40 agonist antibody is a full-length IgG1 antibody.

155. The method of claim 80, wherein the OX40 immunoadhesin is a trimeric OX40-Fc protein.

156. The method of any one of claims 1, 2, 12, 13, 23-24, and 49-155, wherein the cancer is selected from the group consisting of non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies.

157. The method of any one of claims 1-11 and 42-156, wherein the agent that decreases or inhibits TIGIT expression and/or activity is administered continuously.

158. The method of any one of claims 1-11 and 42-156, wherein the agent that decreases or inhibits TIGIT expression and/or activity is administered intermittently.

159. The method of any one of claims 1-11 and 42-158, wherein the agent that decreases or inhibits TIGIT expression and/or activity is administered before the OX40 binding agonist.

160. The method of any one of claims 1-11 and 42-158, wherein the agent that decreases or inhibits TIGIT expression and/or activity is administered simultaneous with the OX40 binding agonist.

161. The method of any one of claims 1-11 and 42-158, wherein the agent that decreases or inhibits TIGIT expression and/or activity is administered after the OX40 binding agonist.

162. The method of any one of claims 12-41 and 49-156, wherein the OX40 binding agonist is administered before the agent that modulates CD226 expression and/or activity.

163. The method of any one of claims 12-41 and 49-156, wherein the OX40 binding agonist is administered simultaneous with the agent that modulates CD226 expression and/or activity.

164. The method of any one of claims 12-41 and 49-156, wherein the OX40 binding agonist is administered after the agent that modulates CD226 expression and/or activity.

165. The method of any one of claims 42-44 and 49-156, wherein the agent that decreases or inhibits TIGIT expression and/or activity is administered before the agent that decreases or inhibits one or more additional immune co-inhibitory receptors.

166. The method of any one of claims 42-44 and 49-156, wherein the agent that decreases or inhibits TIGIT expression and/or activity is administered simultaneous with the agent that decreases or inhibits one or more additional immune co-inhibitory receptors.

167. The method of any one of claims 42-44 and 49-156, wherein the agent that decreases or inhibits TIGIT expression and/or activity is administered after the agent that decreases or inhibits one or more additional immune co-inhibitory receptors.

168. The method of any one of claims 45-156, wherein the agent that decreases or inhibits TIGIT expression and/or activity is administered before the agent that increases or activates one or more additional immune co-stimulatory receptors or their ligands.

169. The method of any one of claims 45-156, wherein the agent that decreases or inhibits TIGIT expression and/or activity is administered simultaneous with the agent that increases or activates one or more additional immune co-stimulatory receptors or their ligands.

170. The method of any one of claims 45-156, wherein the agent that decreases or inhibits TIGIT expression and/or activity is administered after the agent that increases or activates one or more additional immune co-stimulatory receptors or their ligands.

171. The method of any one of claims 42-44 and 49-156, wherein the OX40 binding agonist is administered before the agent that decreases or inhibits one or more additional immune co-inhibitory receptors.

172. The method of any one of claims 42-44 and 49-156, wherein the OX40 binding agonist is administered simultaneous with the agent that decreases or inhibits one or more additional immune co-inhibitory receptors.

173. The method of any one of claims 42-44 and 49-156, wherein the OX40 binding agonist is administered after the agent that decreases or inhibits one or more additional immune co-inhibitory receptors.

174. The method of any one of claims 45-156, wherein the OX40 binding agonist is administered before the agent that increases or activates one or more additional immune co-stimulatory receptors or their ligands.

175. The method of any one of claims 45-156, wherein the OX40 binding agonist is administered simultaneous with the agent that increases or activates one or more additional immune co-stimulatory receptors or their ligands.

176. The method of any one of claims 45-156, wherein the OX40 binding agonist is administered after the agent that increases or activates one or more additional immune co-stimulatory receptors or their ligands.

177. A kit comprising an OX40 binding agonist and a package insert comprising instructions for using the OX40 binding agonist in combination with an agent that decreases or inhibits TIGIT expression and/or activity to treat or delay progression of cancer in an individual.

178. A kit comprising an OX40 binding agonist and an agent that decreases or inhibits TIGIT expression and/or activity, and a package insert comprising instructions for using the OX40 binding agonist and the agent that decreases or inhibits TIGIT expression and/or activity to treat or delay progression of cancer in an individual.

179. A kit comprising an agent that decreases or inhibits TIGIT expression and/or activity and a package insert comprising instructions for using the agent that decreases or inhibits TIGIT expression and/or activity in combination with an OX40 binding agonist to treat or delay progression of cancer in an individual.

180. A kit comprising an OX40 binding agonist and a package insert comprising instructions for using the OX40 binding agonist in combination with an agent that decreases or inhibits TIGIT expression and/or activity to enhance immune function of an individual having cancer.

181. A kit comprising an OX40 binding agonist and an agent that decreases or inhibits TIGIT expression and/or activity, and a package insert comprising instructions for using the OX40 binding agonist and the agent that decreases or inhibits TIGIT expression and/or activity to enhance immune function of an individual having cancer.

182. A kit comprising an agent that decreases or inhibits TIGIT expression and/or activity and a package insert comprising instructions for using the agent that decreases or inhibits TIGIT expression and/or activity in combination with an OX40 binding agonist to enhance immune function of an individual having cancer.

183. A kit comprising an OX40 binding agonist and a package insert comprising instructions for using the OX40 binding agonist in combination with an agent that modulates CD226 expression and/or activity to treat or delay progression of cancer in an individual.

184. A kit comprising an OX40 binding agonist and an agent that modulates CD226 expression and/or activity, and a package insert comprising instructions for using the OX40 binding agonist and the agent that modulates CD226 expression and/or activity to treat or delay progression of cancer in an individual.

185. A kit comprising an agent that modulates CD226 expression and/or activity and a package insert comprising instructions for using the agent modulates CD226 expression and/or activity in combination with an OX40 binding agonist to treat or delay progression of cancer in an individual.

186. A kit comprising an OX40 binding agonist and a package insert comprising instructions for using the OX40 binding agonist in combination with an agent that modulates CD226 expression and/or activity to enhance immune function of an individual having cancer.

187. A kit comprising an OX40 binding agonist and an agent that modulates CD226 expression and/or activity, and a package insert comprising instructions for using the OX40 binding agonist and the agent that modulates CD226 expression and/or activity to enhance immune function of an individual having cancer.

188. A kit comprising an agent modulates CD226 expression and/or activity and a package insert comprising instructions for using the agent that modulates CD226 expression and/or activity in combination with an OX40 binding agonist to enhance immune function of an individual having cancer.
